SEQUENCE LISTING (158 sequences)

SEQ ID NO: 1 - DNA, 17 bp, Artificial Sequence
  Description of Artificial Sequence Synthetic oligonucleotide
aagggggttccttgggc

SEQ ID NO: 2 - DNA, 17 bp, Artificial Sequence
  Description of Artificial Sequence Synthetic oligonucleotide
ggcctgccagaagcaca

SEQ ID NO: 3 - DNA, 17 bp, Artificial Sequence
  Description of Artificial Sequence Synthetic oligonucleotide
gccgattaacaccagcc

SEQ ID NO: 4 - DNA, 17 bp, Artificial Sequence
  Description of Artificial Sequence Synthetic oligonucleotide
cgattaaccaccggcct

SEQ ID NO: 5 - DNA, 17 bp, Artificial Sequence
  Description of Artificial Sequence Synthetic oligonucleotide
ttgaaccctaggcatgt

SEQ ID NO: 6 - DNA, 17 bp, Artificial Sequence
  Description of Artificial Sequence Synthetic oligonucleotide
cctgagcaaacctgagc

SEQ ID NO: 7 - DNA, 17 bp, Artificial Sequence
  Description of Artificial Sequence Synthetic oligonucleotide
cgcatgcgtggccacca

SEQ ID NO: 8 - DNA, 17 bp, Artificial Sequence
  Description of Artificial Sequence Synthetic oligonucleotide
ccaactggatcccaggt

SEQ ID NO: 9 - DNA, 17 bp, Artificial Sequence
  Description of Artificial Sequence Synthetic oligonucleotide
cggatgtccctgctggg

SEQ ID NO: 10 - DNA, 17 bp, Artificial Sequence
  Description of Artificial Sequence Synthetic oligonucleotide
gccgattcacacccagc

SEQ ID NO: 11 - DNA, 16 bp, Artificial Sequence
  Description of Artificial Sequence Synthetic oligonucleotide
gcctcgtacctagccg

SEQ ID NO: 12 - DNA, 17 bp, Artificial Sequence
  Description of Artificial Sequence Synthetic oligonucleotide
cgcctcggccgattaac

SEQ ID NO: 13 - DNA, 17 bp, Artificial Sequence
  Description of Artificial Sequence Synthetic oligonucleotide
ggccgatttacacccgg

SEQ ID NO: 14 - DNA, 17 bp, Artificial Sequence
  Description of Artificial Sequence Synthetic oligonucleotide
tcggccgattaacccca

SEQ ID NO: 15 - DNA, 17 bp, Artificial Sequence
  Description of Artificial Sequence Synthetic oligonucleotide
ccgattaacaccggcct

SEQ ID NO: 16 - DNA, 18 bp, Artificial Sequence
  Description of Artificial Sequence Synthetic oligonucleotide
gctgttgtcatacttgct

SEQ ID NO: 17 - DNA, 17 bp, Artificial Sequence
  Description of Artificial Sequence Synthetic oligonucleotide
ccacgtgatgtagactg

SEQ ID NO: 18 - DNA, 17 bp, Artificial Sequence
  Description of Artificial Sequence Synthetic oligonucleotide
cccagcctcgtacctag

SEQ ID NO: 19 - DNA, 18 bp, Artificial Sequence
  Description of Artificial Sequence Synthetic oligonucleotide
cagcctctacctagcctt

SEQ ID NO: 20 - DNA, 16 bp, Artificial Sequence
  Description of Artificial Sequence Synthetic oligonucleotide
caccggcctcgtacct

SEQ ID NO: 21 - DNA, 17 bp, Artificial Sequence
  Description of Artificial Sequence Synthetic oligonucleotide
gcccaaggaaccccctt

SEQ ID NO: 22 - DNA, 17 bp, Artificial Sequence
  Description of Artificial Sequence Synthetic oligonucleotide
gctcaggtttgctcagg

SEQ ID NO: 23 - DNA, 17 bp, Artificial Sequence
  Description of Artificial Sequence Synthetic oligonucleotide
tgtgcttctggcaggcc

SEQ ID NO: 24 - DNA, 25 bp, Artificial Sequence
  Description of Artificial Sequence Synthetic oligonucleotide
acctgggatccagttggaggacggc

SEQ ID NO: 25 - DNA, 25 bp, Artificial Sequence
  Description of Artificial Sequence Synthetic oligonucleotide
gccgtcctccaactggatcccaggt

SEQ ID NO: 26 - DNA, 17 bp, Artificial Sequence
  Description of Artificial Sequence Synthetic oligonucleotide
tggtggccacgcatgcg

SEQ ID NO: 27 - DNA, 17 bp, Artificial Sequence
  Description of Artificial Sequence Synthetic oligonucleotide
cccagcagggacatccg

SEQ ID NO: 28 - DNA, 18 bp, Artificial Sequence
  Description of Artificial Sequence Synthetic oligonucleotide
ggctaggtacgaggctgg

SEQ ID NO: 29 - DNA, 18 bp, Artificial Sequence
  Description of Artificial Sequence Synthetic oligonucleotide
ccagcctcgtacctagcc

SEQ ID NO: 30 - DNA, 22 bp, Artificial Sequence
  Description of Artificial Sequence Synthetic oligonucleotide
ggctggtgttaatcggccgagg

SEQ ID NO: 31 - DNA, 22 bp, Artificial Sequence
  Description of Artificial Sequence Synthetic oligonucleotide
cctcggccgattaacaccagcc

SEQ ID NO: 32 - DNA, 19 bp, Artificial Sequence
  Description of Artificial Sequence Synthetic oligonucleotide
gggggtgaatcggccgagg

SEQ ID NO: 33 - DNA, 19 bp, Artificial Sequence
  Description of Artificial Sequence Synthetic oligonucleotide
cctcggccgattcaccccc

SEQ ID NO: 34 - DNA, 22 bp, Artificial Sequence
  Description of Artificial Sequence Synthetic oligonucleotide
gctgggtgtgaatcggccgagg

SEQ ID NO: 35 - DNA, 22 bp, Artificial Sequence
  Description of Artificial Sequence Synthetic oligonucleotide
cctcggccgattcacacccagc

SEQ ID NO: 36 - DNA, 19 bp, Artificial Sequence
  Description of Artificial Sequence Synthetic oligonucleotide
aggtacgaggccgggtgtt

SEQ ID NO: 37 - DNA, 19 bp, Artificial Sequence
  Description of Artificial Sequence Synthetic oligonucleotide
aacacccggcctcgtacct

SEQ ID NO: 38 - DNA, 17 bp, Artificial Sequence
  Description of Artificial Sequence Synthetic oligonucleotide
gtgttaatcggccgagg

SEQ ID NO: 39 - DNA, 17 bp, Artificial Sequence
  Description of Artificial Sequence Synthetic oligonucleotide
cctcggccgattaacac

SEQ ID NO: 40 - DNA, 17 bp, Artificial Sequence
  Description of Artificial Sequence Synthetic oligonucleotide
agatgggtaccaactgt

SEQ ID NO: 41 - DNA, 17 bp, Artificial Sequence
  Description of Artificial Sequence Synthetic oligonucleotide
acagttggtacccatct

SEQ ID NO: 42 - DNA, 22 bp, Artificial Sequence
  Description of Artificial Sequence Synthetic oligonucleotide
cggctaggtacgaggctggggt

SEQ ID NO: 43 - DNA, 22 bp, Artificial Sequence
  Description of Artificial Sequence Synthetic oligonucleotide
accccagcctcgtacctagccg

SEQ ID NO: 44 - DNA, 24 bp, Artificial Sequence
  Description of Artificial Sequence Synthetic oligonucleotide
gaggcgggtgtgaatcggccgagg

SEQ ID NO: 45 - DNA, 24 bp, Artificial Sequence
  Description of Artificial Sequence Synthetic oligonucleotide
cctcggccgattcacacccgcctc

SEQ ID NO: 46 - DNA, 17 bp, Artificial Sequence
  Description of Artificial Sequence Synthetic oligonucleotide
aggtacgaggccggtgt

SEQ ID NO: 47 - DNA, 17 bp, Artificial Sequence
  Description of Artificial Sequence Synthetic oligonucleotide
acaccggcctcgtacct

SEQ ID NO: 48 - DNA, 18 bp, Artificial Sequence
  Description of Artificial Sequence Synthetic oligonucleotide
gttaatcggccgaggcgc

SEQ ID NO: 49 - DNA, 18 bp, Artificial Sequence
  Description of Artificial Sequence Synthetic oligonucleotide
gcgcctcggccgattaac

SEQ ID NO: 50 - DNA, 17 bp, Artificial Sequence
  Description of Artificial Sequence Synthetic oligonucleotide
agaccaacagagttcgg

SEQ ID NO: 51 - DNA, 17 bp, Artificial Sequence
  Description of Artificial Sequence Synthetic oligonucleotide
ccgaactctgttggtct

SEQ ID NO: 52 - DNA, 19 bp, Artificial Sequence
  Description of Artificial Sequence Synthetic oligonucleotide
tggcttcgtgtcccatgca

SEQ ID NO: 53 - DNA, 19 bp, Artificial Sequence
  Description of Artificial Sequence Synthetic oligonucleotide
tgcatgggacacgaagcca

SEQ ID NO: 54 - DNA, 19 bp, Artificial Sequence
  Description of Artificial Sequence Synthetic oligonucleotide
ccgggtgtaaatcggccga

SEQ ID NO: 55 - DNA, 19 bp, Artificial Sequence
  Description of Artificial Sequence Synthetic oligonucleotide
tcggccgatttacacccgg

SEQ ID NO: 56 - DNA, 19 bp, Artificial Sequence
  Description of Artificial Sequence Synthetic oligonucleotide
gccggtgtgaatcggccga

SEQ ID NO: 57 - DNA, 19 bp, Artificial Sequence
  Description of Artificial Sequence Synthetic oligonucleotide
tcggccgattcacaccggc

SEQ ID NO: 58 - DNA, 20 bp, Artificial Sequence
  Description of Artificial Sequence Synthetic oligonucleotide
tcatgatggtgtatcgatga

SEQ ID NO: 59 - DNA, 20 bp, Artificial Sequence
  Description of Artificial Sequence Synthetic oligonucleotide
tcatcgatacaccatcatga

SEQ ID NO: 60 - DNA, 20 bp, Artificial Sequence
  Description of Artificial Sequence Synthetic oligonucleotide
gctcggtgttaatcggccga

SEQ ID NO: 61 - DNA, 20 bp, Artificial Sequence
  Description of Artificial Sequence Synthetic oligonucleotide
tcggccgattaacaccgagc

SEQ ID NO: 62 - DNA, 19 bp, Artificial Sequence
  Description of Artificial Sequence Synthetic oligonucleotide
tggggttaatcggccgagg

SEQ ID NO: 63 - DNA, 19 bp, Artificial Sequence
  Description of Artificial Sequence Synthetic oligonucleotide
cctcggccgattaacccca

SEQ ID NO: 64 - DNA, 21 bp, Artificial Sequence
  Description of Artificial Sequence Synthetic oligonucleotide
aggccggtgttaatcggccga

SEQ ID NO: 65 - DNA, 21 bp, Artificial Sequence
  Description of Artificial Sequence Synthetic oligonucleotide
tcggccgattaacaccggcct

SEQ ID NO: 66 - DNA, 19 bp, Artificial Sequence
  Description of Artificial Sequence Synthetic oligonucleotide
tggtgaatcggccgagggt

SEQ ID NO: 67 - DNA, 19 bp, Artificial Sequence
  Description of Artificial Sequence Synthetic oligonucleotide
accctcggccgattcacca

SEQ ID NO: 68 - DNA, 18 bp, Artificial Sequence
  Description of Artificial Sequence Synthetic oligonucleotide
agcaagtatgacaacagc

SEQ ID NO: 69 - DNA, 18 bp, Artificial Sequence
  Description of Artificial Sequence Synthetic oligonucleotide
gctgttgtcatacttgct

SEQ ID NO: 70 - DNA, 17 bp, Artificial Sequence
  Description of Artificial Sequence Synthetic oligonucleotide
cttaaaccaagctagcc

SEQ ID NO: 71 - DNA, 17 bp, Artificial Sequence
  Description of Artificial Sequence Synthetic oligonucleotide
ggctagcttggtttaag

SEQ ID NO: 72 - DNA, 17 bp, Artificial Sequence
  Description of Artificial Sequence Synthetic oligonucleotide
cagtctacatcacgtgg

SEQ ID NO: 73 - DNA, 17 bp, Artificial Sequence
  Description of Artificial Sequence Synthetic oligonucleotide
ccacgtgatgtagactg

SEQ ID NO: 74 - DNA, 18 bp, Artificial Sequence
  Description of Artificial Sequence Synthetic oligonucleotide
aatctcctgttacactca

SEQ ID NO: 75 - DNA, 18 bp, Artificial Sequence
  Description of Artificial Sequence Synthetic oligonucleotide
tgagtgtaacaggagatt

SEQ ID NO: 76 - DNA, 17 bp, Artificial Sequence
  Description of Artificial Sequence Synthetic oligonucleotide
gcccaaggaaccccctt

SEQ ID NO: 77 - DNA, 17 bp, Artificial Sequence
  Description of Artificial Sequence Synthetic oligonucleotide
aagggggttccttgggc

SEQ ID NO: 78 - DNA, 18 bp, Artificial Sequence
  Description of Artificial Sequence Synthetic oligonucleotide
ggctaggacgaggccggg

SEQ ID NO: 79 - DNA, 18 bp, Artificial Sequence
  Description of Artificial Sequence Synthetic oligonucleotide
cccggcctcgtcctagcc

SEQ ID NO: 80 - DNA, 17 bp, Artificial Sequence
  Description of Artificial Sequence Synthetic oligonucleotide
gagaaggttcccgggaa

SEQ ID NO: 81 - DNA, 17 bp, Artificial Sequence
  Description of Artificial Sequence Synthetic oligonucleotide
ttcccgggaaccttctc

SEQ ID NO: 82 - DNA, 17 bp, Artificial Sequence
  Description of Artificial Sequence Synthetic oligonucleotide
gtgttactcggccgagg

SEQ ID NO: 83 - DNA, 17 bp, Artificial Sequence
  Description of Artificial Sequence Synthetic oligonucleotide
cctcggccgagtaacac

SEQ ID NO: 84 - DNA, 18 bp, Artificial Sequence
  Description of Artificial Sequence Synthetic oligonucleotide
ttgaatcggccgagggtg

SEQ ID NO: 85 - DNA, 18 bp, Artificial Sequence
  Description of Artificial Sequence Synthetic oligonucleotide
caccctcggccgattcaa

SEQ ID NO: 86 - DNA, 17 bp, Artificial Sequence
  Description of Artificial Sequence Synthetic oligonucleotide
gccgggtggtgaatcgg

SEQ ID NO: 87 - DNA, 17 bp, Artificial Sequence
  Description of Artificial Sequence Synthetic oligonucleotide
ccgattcaccacccggc

SEQ ID NO: 88 - DNA, 17 bp, Artificial Sequence
  Description of Artificial Sequence Synthetic oligonucleotide
gccggtggttaatcggc

SEQ ID NO: 89 - DNA, 17 bp, Artificial Sequence
  Description of Artificial Sequence Synthetic oligonucleotide
gccgattaaccaccggc

SEQ ID NO: 90 - DNA, 17 bp, Artificial Sequence
  Description of Artificial Sequence Synthetic oligonucleotide
gggcgcagcgacatcag

SEQ ID NO: 91 - DNA, 17 bp, Artificial Sequence
  Description of Artificial Sequence Synthetic oligonucleotide
ctgatgtcgctgcgccc

SEQ ID NO: 92 - DNA, 18 bp, Artificial Sequence
  Description of Artificial Sequence Synthetic oligonucleotide
gctattagcagattgtgt

SEQ ID NO: 93 - DNA, 18 bp, Artificial Sequence
  Description of Artificial Sequence Synthetic oligonucleotide
acacaatctgctaatagc

SEQ ID NO: 94 - DNA, 22 bp, Artificial Sequence
  Description of Artificial Sequence Synthetic oligonucleotide
tgttaatctcctgttacactca

SEQ ID NO: 95 - DNA, 22 bp, Artificial Sequence
  Description of Artificial Sequence Synthetic oligonucleotide
tgagtgtaacaggagattaaca

SEQ ID NO: 96 - DNA, 17 bp, Artificial Sequence
  Description of Artificial Sequence Synthetic oligonucleotide
ccaccgcaccgttggcc

SEQ ID NO: 97 - DNA, 17 bp, Artificial Sequence
  Description of Artificial Sequence Synthetic oligonucleotide
ggccaacggtgcggtgg

SEQ ID NO: 98 - DNA, 17 bp, Artificial Sequence
  Description of Artificial Sequence Synthetic oligonucleotide
acctggagccctctgat

SEQ ID NO: 99 - DNA, 17 bp, Artificial Sequence
  Description of Artificial Sequence Synthetic oligonucleotide
atcagagggctccaggt

SEQ ID NO: 100 - DNA, 18 bp, Artificial Sequence
  Description of Artificial Sequence Synthetic oligonucleotide
tcagacaaacacagatcg

SEQ ID NO: 101 - DNA, 18 bp, Artificial Sequence
  Description of Artificial Sequence Synthetic oligonucleotide
cgatctgtgtttgtctga

SEQ ID NO: 102 - DNA, 21 bp, Artificial Sequence
  Description of Artificial Sequence Synthetic oligonucleotide
gagaatactgattgagaccta

SEQ ID NO: 103 - DNA, 21 bp, Artificial Sequence
  Description of Artificial Sequence Synthetic oligonucleotide
taggtctcaatcagtattctc

SEQ ID NO: 104 - DNA, 17 bp, Artificial Sequence
  Description of Artificial Sequence Synthetic oligonucleotide
ccagccagcacccaggc

SEQ ID NO: 105 - DNA, 17 bp, Artificial Sequence
  Description of Artificial Sequence Synthetic oligonucleotide
gcctgggtgctggctgg

SEQ ID NO: 106 - DNA, 17 bp, Artificial Sequence
  Description of Artificial Sequence Synthetic oligonucleotide
tagaccaacagagttcc

SEQ ID NO: 107 - DNA, 17 bp, Artificial Sequence
  Description of Artificial Sequence Synthetic oligonucleotide
ggaactctgttggtcta

SEQ ID NO: 108 - DNA, 21 bp, Artificial Sequence
  Description of Artificial Sequence Synthetic oligonucleotide
ctaggtacgaggctgggtttt

SEQ ID NO: 109 - DNA, 21 bp, Artificial Sequence
  Description of Artificial Sequence Synthetic oligonucleotide
aaaacccagcctcgtacctag

SEQ ID NO: 110 - DNA, 21 bp, Artificial Sequence
  Description of Artificial Sequence Synthetic oligonucleotide
cgaggcgggtgttaatcggcc

SEQ ID NO: 111 - DNA, 21 bp, Artificial Sequence
  Description of Artificial Sequence Synthetic oligonucleotide
ggccgattaacacccgcctcg

SEQ ID NO: 112 - DNA, 18 bp, Artificial Sequence
  Description of Artificial Sequence Synthetic oligonucleotide
aaggctaggtagaggctg

SEQ ID NO: 113 - DNA, 18 bp, Artificial Sequence
  Description of Artificial Sequence Synthetic oligonucleotide
cagcctctacctagcctt

SEQ ID NO: 114 - DNA, 17 bp, Artificial Sequence
  Description of Artificial Sequence Synthetic oligonucleotide
catggccatgctgtgca

SEQ ID NO: 115 - DNA, 17 bp, Artificial Sequence
  Description of Artificial Sequence Synthetic oligonucleotide
tgcacagcatggccatg

SEQ ID NO: 116 - DNA, 28 bp, Artificial Sequence
  Description of Artificial Sequence Synthetic oligonucleotide
aggtacgaggccggtgttaatcggccga

SEQ ID NO: 117 - DNA, 28 bp, Artificial Sequence
  Description of Artificial Sequence Synthetic oligonucleotide
tcggccgattaacaccggcctcgtacct

SEQ ID NO: 118 - DNA, 17 bp, Artificial Sequence
  Description of Artificial Sequence Synthetic oligonucleotide
tgctgccctcaatggtc

SEQ ID NO: 119 - DNA, 17 bp, Artificial Sequence
  Description of Artificial Sequence Synthetic oligonucleotide
gaccattgagggcagca

SEQ ID NO: 120 - DNA, 24 bp, Artificial Sequence
  Description of Artificial Sequence Synthetic oligonucleotide
aggccggtggttaatcggccgagg

SEQ ID NO: 121 - DNA, 24 bp, Artificial Sequence
  Description of Artificial Sequence Synthetic oligonucleotide
cctcggccgattaaccaccggcct

SEQ ID NO: 122 - DNA, 24 bp, Artificial Sequence
  Description of Artificial Sequence Synthetic oligonucleotide
gaggccggtggttaatcggccgag

SEQ ID NO: 123 - DNA, 24 bp, Artificial Sequence
  Description of Artificial Sequence Synthetic oligonucleotide
ctcggccgattaaccaccggcctc

SEQ ID NO: 124 - DNA, 22 bp, Artificial Sequence
  Description of Artificial Sequence Synthetic oligonucleotide
gctaggtacgaggctgggtttt

SEQ ID NO: 125 - DNA, 22 bp, Artificial Sequence
  Description of Artificial Sequence Synthetic oligonucleotide
aaaacccagcctcgtacctagc

SEQ ID NO: 126 - DNA, 19 bp, Artificial Sequence
  Description of Artificial Sequence Synthetic oligonucleotide
aacatacggctaggtacga

SEQ ID NO: 127 - DNA, 19 bp, Artificial Sequence
  Description of Artificial Sequence Synthetic oligonucleotide
tcgtacctagccgtatgtt

SEQ ID NO: 128 - DNA, 18 bp, Artificial Sequence
  Description of Artificial Sequence Synthetic oligonucleotide
ggtggtaatcggacgagg

SEQ ID NO: 129 - DNA, 18 bp, Artificial Sequence
  Description of Artificial Sequence Synthetic oligonucleotide
cctcgtccgattaccacc

SEQ ID NO: 130 - DNA, 17 bp, Artificial Sequence
  Description of Artificial Sequence Synthetic oligonucleotide
gggtgatcggacgaggc

SEQ ID NO: 131 - DNA, 17 bp, Artificial Sequence
  Description of Artificial Sequence Synthetic oligonucleotide
gcctcgtccgatcaccc

SEQ ID NO: 132 - DNA, 17 bp, Artificial Sequence
  Description of Artificial Sequence Synthetic oligonucleotide
acatgcctagggttcaa

SEQ ID NO: 133 - DNA, 62 bp, Homo sapiens
tgccctccacaggactctccctactgcctgagcaaacctgagcctcccggcagacccaccca

SEQ ID NO: 134 - DNA, 61 bp, Homo sapiens
tgccctccacagactctccctactgcctgagcaaacctgagcgtcccggcagacccaccca

SEQ ID NO: 135 - DNA, 62 bp, Homo sapiens; identical to SEQ ID NO: 133

SEQ ID NO: 136 - DNA, 62 bp, Homo sapiens
tgccctccacaggactctccctactgcctgagcaaacctgagcgtcccggcagacccaccca

SEQ ID NO: 137 - DNA, 62 bp, Homo sapiens; identical to SEQ ID NO: 136

SEQ ID NO: 138 - DNA, 63 bp, Homo sapiens
tgccctccacaggactctccctactgcctgagcaaacctgagcgctcccggcagacccaccca

SEQ ID NO: 139 - DNA, 63 bp, Homo sapiens
tgccctccacagtactctccctactgcctgagcaaacctgagcgctcccggcagacccaccca

SEQ ID NO: 140 - DNA, 55 bp, Homo sapiens
tgcctcacagatctcctactcctgagcaaacctgagcgtccggcagacccaccca

SEQ ID NO: 141 - DNA, 63 bp, Homo sapiens
tgccctccacaggactctccctactgcctgagcaaacctgaggcctcccggcagacccaccca

SEQ ID NO: 142 - DNA, 52 bp, Homo sapiens
ctggcctgagaagttctgcacatgcgtggcaccatttcctgtgaacacttgc

SEQ ID NO: 143 - DNA, 52 bp, Homo sapiens
ccagcccgggaaggtttgcgcgcgtgtggcaccatttcccatgaacacccat

SEQ ID NO: 144 - DNA, 52 bp, Homo sapiens
ctggctcgggaagttctgcgcatgcgtggcaccatttcccgtgaacacccat

SEQ ID NO: 145 - DNA, 52 bp, Homo sapiens
ctggctcgggaagttctgcgcatgcgtggcaccatttctcgtgaacacctat

SEQ ID NO: 146 - DNA, 52 bp, Homo sapiens
ctggctcgggaagttctgcgcatgcgtggcaccgtttcccgtgaacacccgt

SEQ ID NO: 147 - DNA, 52 bp, Homo sapiens
ctggcttgggaagttctgcgcatgcatggcaccatttcccgtgaacacccat

SEQ ID NO: 148 - DNA, 32 bp, Homo sapiens
aagttctgcgcatgcgtggcaccatttctcgt

SEQ ID NO: 149 - DNA, 32 bp, Homo sapiens
aagttctgcgcatgcgtggcaccatttcccgt

SEQ ID NO: 150 - DNA, 32 bp, Homo sapiens; identical to SEQ ID NO: 149

SEQ ID NO: 151 - DNA, 32 bp, Homo sapiens
aagttctgcgcatgcgtggcaccatttcctgt

SEQ ID NO: 152 - DNA, 30 bp, Homo sapiens
gtactgtgcatgcgtggcaccatttcccgt

SEQ ID NO: 153 - DNA, 30 bp, Homo sapiens
gttctgtgcatgcgtggcaccatttcccgt

SEQ ID NO: 154 - DNA, 32 bp, Homo sapiens
aagttctgcacatgcgtggcaccatttcctgt

SEQ ID NO: 155 - DNA, 32 bp, Homo sapiens
aagttctgcgcatgtgtggcaccatttcccgt

SEQ ID NO: 156 - DNA, 30 bp, Homo sapiens
gttctgtgcatgcttggcaccatttcctgt

SEQ ID NO: 157 - DNA, 32 bp, Homo sapiens
aagttctgcacatgtgtggcaccatttcctgt

SEQ ID NO: 158 - DNA, 32 bp, Homo sapiens
aagttttgcgcatgcgtgacaccatttcccat

(19) United States
(12) Patent Application Publication
(10) Pub. No.: US 2008/0045471 A1
(43) Pub. Date: Feb. 21, 2008

(54) Nucleic Acids For Apoptosis Of Cancer Cells

(76) Inventor: Don Adams North, Arlington, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/691,994

(22) Filed: Mar. 27, 2007

Related U.S. Application Data
(60) Provisional application No. 60/786,316, filed on Mar. 27, 2006.
(60) Provisional application No. 60/820,577, filed on Jul. 27, 2006.

(51) Int. Cl. A61K 48/00; C07H 21/00; C12N 5/06

(52) U.S. Cl. 514/44; 435/375; 536/23.1

(57) ABSTRACT

The disclosure relates to nucleic acids having Apoptotic Sequence Nos. 5, 8, 9, 11, 14, 60 and 66. It also relates to agents targeting Apoptotic Sequences, said agents having SEQ ID NO:1, SEQ ID NO:2, SEQ ID NO:3, SEQ ID NO:4, SEQ ID NO: 5, SEQ ID NO:6, and SEQ ID NO:7. The composition may also include a pharmaceutically acceptable carrier. The disclosure also includes a method of killing a cancer cell by administering to a cancer cell a treatment formulation including a nucleic acid having an Apoptotic Sequence targeting agent of: SEQ ID NO:1, SEQ ID NO:2, SEQ ID NO:3, SEQ ID NO:4, SEQ ID NO: 5, SEQ ID NO:6, and SEQ ID NO:7 and a pharmaceutically acceptable carrier. The cancer cell may be located in a subject with cancer.

### PRIORITY CLAIM

The present application claims priority under 35 U.S.C. §119(e) to U.S. Provisional Patent Application Serial No. Ser. No. 60/786,316, filed Mar. 27, 2006, titled “Gene Targeting-Induced Apoptosis of Cancer Cells,” and to U.S. Provisional Patent Application Ser. No. 60/820,577, filed Jul. 27, 2006, titled “Nucleic Acid Targeted Cancer Cell Death Agents,” both of which are incorporated by reference herein in their entireties.

### TECHNICAL FIELD

The present invention, in some embodiments, relates to a subset of Cancer Marker Sequences termed Apoptotic Sequences found in particular cancer specific mutations. These unique Apoptotic Sequences provide targets for the action of suitable targeting agents, which cause induction of cell death in cancer cells while leaving healthy cells unharmed. The present invention, in some embodiments, provides for targeting agents whose design or activity is based on knowledge of Apoptotic Sequences. Other embodiments of the invention also relate to targeting agents, particularly oligonucleotides, which induce death in cancer cells using nucleic acid sequence information from the Cancer Marker or Apoptotic Sequences.

### BACKGROUND

Cancer results when a cell in the body malfunctions and begins to replicate abnormally. The safest, most effective cancer treatments kill cancer cells without significantly harming healthy cells. This relies upon distinguishing cancer cells from healthy cells, which current methods of chemotherapy and radiotherapy do quite poorly.

Much cancer research focuses on emergence of oncogenes and inactivating mutations of tumor suppressor genes because these genes have a clearly delineated association with abnormal cell replication. However, addressing tumor therapy to these types of genes has only been modestly effective. There remains in the art a need to find effective cancer therapies that have minimal toxicity and other adverse effects.

### SUMMARY

In one embodiment, the invention provides nucleic acids, particularly oligonucleotides that are found in cancer cell but not normal cell transcriptomes. These mutations, unique to cancer cells are termed “Cancer Marker Sequences” in the context of this invention. In an alternative embodiment, the present invention provides for “Apoptotic Sequences.” Apoptotic Sequences are a subset of the cancer cell transcriptome-specific Cancer Marker Sequences. Administration of agents derived from one or more Apoptotic Sequences, i.e. targeting agents, induces growth inhibition or death of cancer cells, through apoptosis or other cell death-inducing mechanisms such as e.g., necrosis. In specific embodiments, the cancer therapeutic targeting agent, based on an Apoptotic Sequence with demonstrated ability to kill cancer cells has a sequence of: SEQ ID NO:1, SEQ ID NO:2, SEQ ID NO:3, SEQ ID NO:4, or SEQ ID NO:5, SEQ ID NO:6, SEQ ID NO:7. In another embodiment of the invention, the cancer therapeutic is targeted based on an Apoptotic Sequence with demonstrated ability to kill cancer cells that is specifically not a sequence of: SEQ ID NO:1, SEQ ID NO:2, SEQ ID NO:3, SEQ ID NO:4, SEQ ID NO:5, SEQ ID NO:6, and/or SEQ ID NO:7.

In other embodiments, Apoptotic Sequences of the invention encode RNA that target genes containing Cancer Marker Sequences, by antisense RNA, interfering RNA (RNAi) or Ribozyme mechanisms. Alternatively, the nucleic acid may be an oligonucleotide, particularly one that uses non-phosphodiester base linkages and is thus resistant to in vivo degradation by endogenous exo- and endonucleases. Such oligonucleotides can be prepared using deoxyribo- or ribo-nucleotide moieties. Another embodiment relates to a composition including a nucleic acid, e.g., having a sequence of SEQ ID NO:1, SEQ ID NO:2, SEQ ID NO:3, SEQ ID NO:4, SEQ ID NO:5, SEQ ID NO:6, SEQ ID NO:7. The invention also provides for a composition that targets an Apoptotic Sequence, that is specifically not a sequence of: SEQ ID NO:1, SEQ ID NO:2, SEQ ID NO:3, SEQ ID NO:4, SEQ ID NO:5, SEQ ID NO:6, or SEQ ID NO:7 and a pharmaceutically acceptable carrier. The cancer cell may be located in a subject with cancer. The composition may also include a pharmaceutically acceptable carrier.

Yet another embodiment relates to a method of killing a cancer cell by administering to a cancer cell a pharmaceutical composition including a nucleic acid e.g., an oligonucleotide targeting agent, that targets an Apoptotic Sequence, such as a sequence of: SEQ ID NO:1, SEQ ID NO:2, SEQ ID NO:3, SEQ ID NO:4, SEQ ID NO:5, SEQ ID NO:6, or SEQ ID NO:7 and a pharmaceutically acceptable carrier. In yet another embodiment, the invention provides for a method of killing a cancer cell by administering to a cancer cell a pharmaceutical composition including a nucleic acid e.g., an oligonucleotide targeting agent, that targets an Apoptotic Sequence, that is specifically not a sequence of: SEQ ID NO:1, SEQ ID NO:2, SEQ ID NO:3, SEQ ID NO:4, SEQ ID NO:5, SEQ ID NO:6, or SEQ ID NO:7 and a pharmaceutically acceptable carrier. The cancer cell may be located in a subject with cancer.

Embodiments of the present invention may be better understood through reference to the following Figures and Detailed Description.

### BRIEF DESCRIPTION OF THE DRAWINGS

FIG. 1 illustrates a Cancer Marker Sequence according to an embodiment of the present invention found in the LTBR gene as aligned to the mRNA from healthy cell transcriptomes (SEQ ID NOS: 133-141). The location of a single nucleotide polymorphism (SNP) is indicated.

FIG. 2 illustrates a Cancer Marker Sequence according to an embodiment of the present invention from six different cancer cell lines and four different cancer types, aligned to the corresponding healthy mRNA from 17 different genes (SEQ ID NOS: 7 (also an Apoptotic Sequence), and SEQ ID NOS:142-158).

FIG. 3 illustrates a method of determining whether a candidate sequence is a Cancer Marker Sequence. The common mutant DNA sequence shown is SEQ ID NO:7.

FIG. 4 illustrates the Cancer Marker Sequence of FIG. 2 (SEQ ID NOS:7 and 26) in multiple cancer cell lines. PCR conditions for isolation of the sequence are also indicated.

FIG. 5 illustrates a method for single-priming PCR using a Cancer Marker Sequence primer, such as a primer having an Apoptotic Sequence according to an embodiment of the present invention.

FIG. 6 presents the results of single-priming PCR as analyzed on gels for cDNA from a healthy human and from tumor or blood samples of two cancer subjects, for various candidate Cancer Marker Sequence derived Apoptotic Sequence-based primers. Apoptotic Sequence-based primers of the present invention include those identified by numbers 5, 8, 9, 11, 14, 60 and 66.

FIG. 7 diagrams how four Apoptotic Sequence nucleic acid related primers, such as DNA (SEQ ID NOS: 5, 1, 4, and 2), of the present invention may be combined in an example embodiment to eradicate cancer cells with four unique cancer mutations.

FIG. 8 presents results following intra-tumoral injection of a therapeutic targeting agent corresponding to Apoptotic Sequence 5 on growth of SW480 human colon carcinoma cells in a nude mouse xenograft. Tumor cross-sectional area (mm²) is plotted against time (days) after intratumoral injection of Apoptotic Sequence 5 based targeting oligonucleotide, PBS or control scrambled sequence.

FIG. 9 is a photograph of an agarose gel visualizing electrophoresed PCR amplified DNA products. The amplified DNAs derived from total RNA templates correspond to control normal tissue, tumor tissue and blood of a colon cancer patient. PCR was performed on cDNA templates reverse transcribed from total RNA, and PCR amplified for 35 cycles. The gel was loaded with amplified DNA samples as follows: Molecular weight standard (lane M), Control (lane 1), colon cancer patient tumor sample (lane 2), blood (lane 3).

FIG. 10 presents the results of intra-tumoral injection of oligonucleotides corresponding to Apoptotic Sequences 5, 9, 60, 66 targeting agents alone, or in combination, into mouse xenografts. The cross sectional tumor volume expressed in mm² was measured over a period of 35 days.

### DETAILED DESCRIPTION

The present invention provides, in one aspect, for treating cancer by targeting Apoptotic Sequences that uniquely characterize cancer cells, which are a subset of Cancer Marker Sequences. The invention is based, in part, on the discovery that oligonucleotides complementary to a subset of Cancer Marker Sequences selectively inhibit the growth of, and more particularly induce cell death of, cancer cells. These observations were made in vitro in tissue culture, and confirmed in vivo, using a mouse model of cancer. Furthermore, the mouse studies demonstrated that at doses much greater than would ordinarily be used therapeutically (and greater than required for efficacy), the oligonucleotides based on Apoptotic Sequence targets had no apparent toxicity.

Cancer Marker Sequences

Embodiments of the present invention relates to Cancer Marker Sequences comprising short nucleic acids having sequences corresponding to cancer-associated mutations present only in the transcriptome (see definition infra) of cancer cells and not in normal cells. Cancer Marker Sequences represent a special kind of cancer mutation—one that has nucleic acid content exclusive to cancer cells. If such exclusivity were not present, the mutation would not be considered a Cancer Marker Sequence. Without such differences, it is not possible to target cancer cells while avoiding healthy cells. Thus a Cancer Marker Sequence provides a target for therapeutic intervention. Cancer Marker Sequences include both nucleic acids having a sequence identical to that of the mutant mRNA and the complementary sequence. However, both complementary nucleic acids are not required for all aspects of this invention. In some aspects, only one or the other of the complementary nucleic acids will be used. The appropriate nucleic acids to use as the Cancer Marker Sequence for each application will be apparent to one skilled in the art.

Many genes may be associated with each Cancer Marker Sequence—the number of genes is normally in direct correlation to the number of unique mRNA molecules containing each Cancer Marker Sequence. Sometimes, hundreds of mRNA molecules contain a Cancer Marker Sequence, yielding hundreds of mapped genes. This is evident in TABLE 1 of U.S. Provisional Patent Application No. 60/742,699 filed Mar. 23, 2006, incorporated herein by reference in its entirety.

TABLE 1 in U.S. Provisional Patent Application No. 60/742,699 filed Mar. 23, 2006, lists Cancer Marker Sequences and the associated cancers. These sequences may include SNPs, but also include longer mutations suitable for diagnostic and targeted cancer cell death use. Cancer Marker Sequences may be utilized in cancer detection and diagnosis. Such sequences, termed Cancer Detection Reagents and their method of use for cancer detection is disclosed in U.S. Provisional Patent Application No. 60/747,260 filed May 15, 2006, incorporated herein by reference in its entirety. TABLE 1 in U.S. Provisional Patent Application No. 60/747,260 filed May 15, 2006, lists Cancer Detection Reagents and the associated cancers.

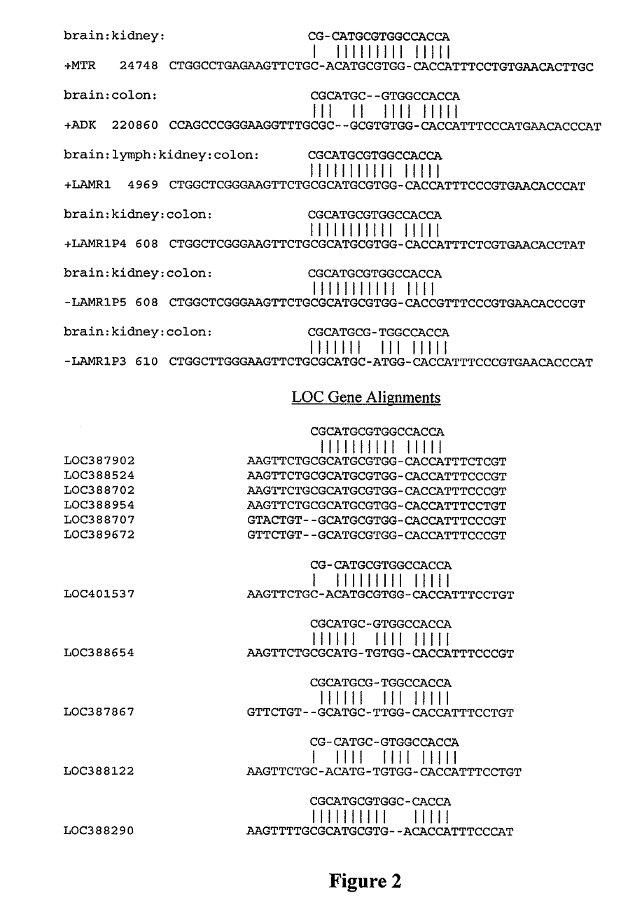

While many of the Cancer Marker Sequences are located in genes with no currently known relevance to cancer, some are located in genes known to be important in cancer. These sequences often represent SNPs (Single Nucleotide Polymorphisms), cryptic splicing and other genetic defects. Cancer Marker Sequences may be common to many genes and many cancers. This does not mean that every Cancer Marker Sequence will exist in every cancer cell line or cancer subject. This is demonstrated in FIG. 2 for six different cancer cell lines and four different cancer types in which they occur.

Apoptotic Sequences

In one embodiment, the present invention relates to nucleic acid sequences having therapeutic properties, which are able to induce cell death in cancer cells while leaving healthy cells unaffected by such criteria as general health and behavior of a test animal or recipient of such nucleic acids. Nucleic acid sequences possessing the above therapeutic cancer cell death inducing property are referred to in the invention as “targeting agents” to Apoptotic Sequences. The design and activity of targeting agents is based on knowledge of Apoptotic Sequences. The cancer-specific Apoptotic Sequences are a subset of Cancer Marker Sequences described above, the targeting of which by a targeting agent results in the induction of cancer cell death. In another embodiment, the invention relates to methods of inducing cell death in cancer cells, for the treatment of cancer using therapeutic pharmaceutical compositions comprising one or more nucleic acid targeting agents each possessing a sequence of a distinct Apoptotic Sequence. Cell death may proceed through apoptosis or through other cell death mechanisms.

Current cancer research focuses on oncogenes and tumor suppressor genes, which are often mutated in cancer cells, but not in normal cells. However, not all DNA abnormalities associated with cancer are located in an oncogene or a tumor suppressor gene. Apoptotic Sequences of the present invention are found in the transcriptome of cancer cells, but are generally absent from that of healthy cells. The transcriptome is the set of all mRNA molecules (or transcripts) in one or a population of biological cells sharing a common lineage, differentiation status, tissue type or environmental circumstances. Therefore, unlike the genome, which is fixed for a given species (apart from genetic polymorphisms), the transcriptome varies depending upon the cellular nature, context or environment.

The location within the genome of Apoptotic Sequences identified in this application is not of primary concern. Some are in tumor suppressor genes or oncogenes, while others are not. However, including nucleic acid sequences based on their differential occurrence in the transcriptome of cancer cells instead of genomic location avoids unnecessarily limiting relevant sequences that may result in a reduction of treatment efficacy. Further, by selecting sequences that are not located in the healthy transcriptome, therapeutics based on targeting those sequences has little or no toxicity to normal cells by gross evaluation of recipients of the treatment.

A single Apoptotic Sequence can represent a common cancer mutation in multiple genes (see Example 11). Thus, the function of an Apoptotic Sequence may not depend on the expression level of a single gene, but may instead benefit from expression of multiple genes at varying levels. In this situation, a single Apoptotic Sequence affects a wide variety of cancer cells. Coupled with the low or non-existent level of harm to normal cells, this allows identification and specific destruction of cancer cells even in samples having relatively low numbers of cancer cells, such as metastasized cells in blood.

Further, the repetitive occurrence of cancer mutation sequences in multiple genes may allow the simultaneous disruption of protein production from these genes. For example, cancer cell death may result from ribosomal protein deficiency.

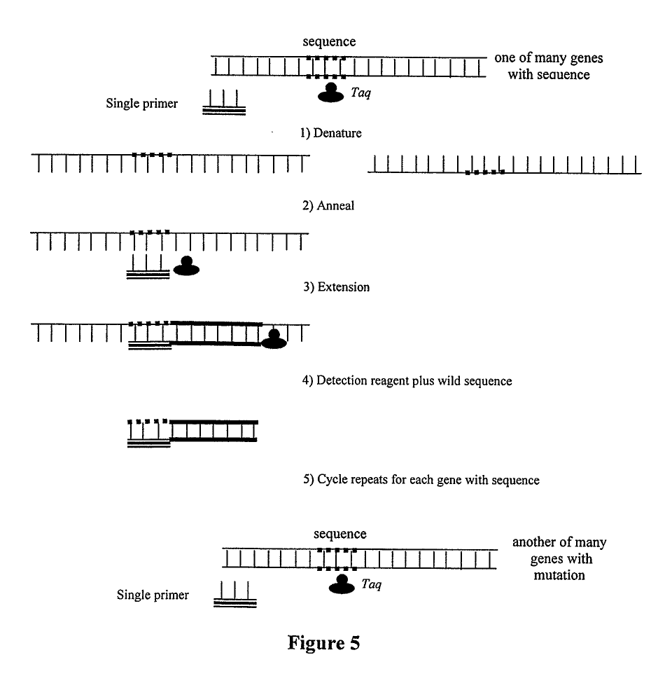

In the same manner that a target cancer mutation can repetitively occur in multiple genes, they can also repetitively occur in multiple cancer types. An Apoptotic Sequence is therefore not necessarily cancer type specific, although each one may have a higher presence in a single cancer type, and/or in one individual subject over another. As a result, it may be desirable to develop a cancer profile for a subject or sample prior to attempting destruction of cancer cells, such as by treatment. This profiling is easily facilitated e.g., using a 20 ml blood sample and the therapeutic nucleic acid including an Apoptotic Sequence based targeting agent as RT-PCR primers. One method of using primers derived from Apoptic Sequences is shown in FIG. 5. Biopsies and other samples may be tested, but are not normally required. As a more convenient alternative, the presence of Apoptotic Sequences may be detected in the metastasized cancer cells present in a subject's blood, which then assures their presence in all or most of the subject's tumors.

Exemplary nucleic acid reagents targeted to an Apoptotic Sequence according to the present invention include but are not limited to a siRNA, a ribozyme, or an antisense molecule and may be between 6-10, 6-20, 6-30, 6-40, 6-50, 6-60, 6-70, 6-80, 6-90 and 6-100 nucleotides in length. Longer sequences can also be used. In a specific embodiment, the Apoptotic Sequence based therapeutic (i.e. targeting agent), particularly when utilized as an oligonucleotide in antisense orientation, is 17 nucleotides in length (Examples 6 and 8-10; SEQ ID NOS:1-7).

General Activity of Agents Targeting Apoptotic Sequences

To further enhance destruction of cancer cells following treatment, Cancer Marker Sequences located in cancer cell transcriptomes were selected for their ability to induce cell death in cells when delivered as a therapeutic nucleic acid to cancer cells (Example 6, Table 3D-E). These Apoptotic Sequences when used as therapeutic targets are associated with the capacity to induce cancer cell death. This is not to say that they are necessarily associated with apoptotic genes, but rather the Apoptotic Sequences themselves, when embodied in a nucleic acid, can trigger cell death.

An Apoptotic Sequence may be present in many genes. Thus an agent targeted to that Apoptotic Sequence can simultaneously interfere with expression of all of these genes harboring a particular target cancer mutation suffocates or starve the cell through a mass protein deficiency. This is different from programmed cell death normally associated with apoptosis. Thus two alternative commonly recognized types of cell death may ensue (see below) following treatment with an Apoptotic Sequence derived targeting agent embodied in a nucleic acid.

Nucleic acids (targeting agents) having Apoptotic Sequences are able to induce cell death of cancer cells as demonstrated by tumor regression in nude mouse xenografts (see Examples 8 and 10). First, the Apoptotic Sequence targeting nucleic acids may be introduced into the cancer cells by uptake from the environment and/or production within the cell. Next, the Apoptotic Sequence targeting nucleic acids may interfere with cellular production of protein, for example by hybridizing with homologous mRNA. This may result in antisense, silencing, or interfering effects, among others.

Induction of Cancer Cell Death by Apoptotic and Non-Apoptotic Mechanisms

The present invention provides for several types of agents in various functional embodiments, which target Apoptotic Sequences, all of which have a direct or indirect activity in mediating cancer cell death. Cell death is generally classified into two categories, programmed cell death or apoptosis, which has an active, well-defined underlying mechanism involving caspases, and non-apoptotic death, or necrosis, which occurs without clearly defined underlying regulatory mechanisms and non-involvement of caspases (Kitanaka et al., Cell Death and Differ. 1999; 6:508-515). Treatment with an agent that targets an Apoptotic Sequence results in cancer cell death. Induction of cell death may involves classically recognized apoptotic cell death end-points such as DNA laddering, Annexin V positive staining, nuclear disintegration etc., or death via non-classical cell death mechanisms, e.g., necrotic death. Embodiments of the present invention provide for an agent which targets an Apoptotic Sequence, wherein said targeting causes cell death. Either apoptotic or non-apoptotic mechanism may be involved. Methods of detection and quantitation of cell death by either mechanism (see below) are envisioned.

Apoptosis is characterized by many biological and morphological changes such as, change of mitochondrial membrane potential, activation of caspases, DNA fragmentation, membrane blebbing and formation of apoptotic bodies. Based on these changes, various assays are designed to detect or quantitate apoptotic cells. Typical assays include Annexin-V binding, caspase enzyme activity measurements, TUNEL (Terminal deoxynucleotidyl transferase-mediated dUTP nick-end-labeling) and DNA gel electrophoresis for DNA laddering. In addition, some of the aforesaid assays have been adapted to measure apoptosis in situ and in vivo (Yang et al., Cancer Biother. Radiopharm. 2001; 16(1):73-83). Embodiments of the present invention provide for but are not limited to the use of any one or more of the above assay methods to detect the presence of, and quantitate apoptosis as a result of exposing a cell population to an Apoptotic Sequence targeting agent. Apoptotic levels may be measured on an isolated homogenous or non-homogenous cell population derived either from in vitro cultured cells or cells derived from or within a subject.

Apoptotic death is associated with nuclear condensation and pyknosis (chromatin condensation) which is generally absent in necrotic (non-apoptotic) death. Methods to distinguish apoptotic from non-apoptotic death are based on morphological and biochemical criteria (Kitanaka et al., Cell Death Differ. 1999; 6:508-515). Apoptosis is accompanied by reduced cytoplasmic volume while necrotic death is accompanied by appearance of autophagic vacuoles, general disintegration and dilation of organelles (Kitanaka et al., supra). When non-apoptotic mechanisms are associated with cell-death induction following treatment with an Apoptotic Sequence targeting agent, embodiments of the present invention provide for methods known in the art to identify and quantitate such non-apoptotic cell-death.

Compositions Targeted to Apoptotic Sequences

Embodiments of the present invention further provide for administration of an agent that targets an Apoptotic Sequence in isolated cells or to a subject in need of such treatment. The present invention provides for treatment of cancer in subjects including but not limited to humans, domestic pets including but not limited to cats, dogs, hamsters, etc., sport and farm animals including but not limited to horses, cattle, sheep etc.

The agents targeted to Apoptotic Sequences may be administered to a subject in accordance with the methods of treatment in an amount sufficient to produce a therapeutic effect (see Examples 6, 8-10 and below). The Apoptotic Sequence targeting-oligonucleotides of the subject invention can be administered to such human or other animal in a conventional dosage form prepared by combining the oligonucleotide of the invention with a conventional, pharmaceutically acceptable carrier, diluent, and/or excipient according to known techniques. It will be recognized by one of ordinary skill in the art that the form and character of the pharmaceutically acceptable carrier, diluent, and/or excipient is dictated by the amount of active ingredient with which it is to be combined, the route of administration, and other well-known variables.

In another aspect, the present invention may provide a composition, e.g., a pharmaceutical composition, containing one or a combination of agents targeted to Apoptotic Sequences, formulated together with a pharmaceutically acceptable carrier. Such compositions may include one or a combination of agents e.g., targeted to two or more different Apoptotic Sequences.

The route of administration of the agent targeted to an Apoptotic Sequence may be oral, parenteral, transmucosal, by inhalation, or topical. The term parenteral as used herein includes intravenous, intramuscular, subcutaneous, rectal, vaginal, or intraperitoneal administration. The subcutaneous, intravenous, and intramuscular forms of parenteral administration are generally preferred. The term transmucosal as used herein includes nasal, buccal, pharyngeal, rectal, vaginal, and ocular.

In one embodiment, the invention provides a therapeutic composition comprising a combination of agents targeted to Apoptotic Sequences which bind to different Cancer Marker Sequences in cancer cells and have complementary cell death inducing activities.

In another embodiment, pharmaceutical compositions of the invention also can be administered in combination therapy, i.e., combined with other agents. For example, the combination therapy can include a composition of the present invention with at least one other therapy such as radio- or chemo-therapy.

As used herein, “pharmaceutically acceptable carrier” includes any and all solvents, dispersion media, coatings, antibacterial and antifungal agents, isotonic and absorption delaying agents, and the like that are physiologically compatible. The carrier may be suitable for intravenous, intramuscular, subcutaneous, parenteral, spinal or epidermal administration (e.g., by injection or infusion). Depending on the route of administration, the active oligonucleotide may be coated in a material to protect it from the action of alkali, acid and other natural conditions that may cause degradation of the oligonucleotide.

A composition of the present invention can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In specific embodiments, the agent that targets an Apoptotic Sequence may be administered to a subject in an appropriate carrier or vector formulation, for example, liposomes, viral capsid, nanoparticle, protein translocation domain, etc., suspended in appropriate pharmaceutical carriers and/or diluents (as described in “delivery systems” below). Pharmaceutically acceptable diluents include saline and aqueous buffer solutions. Liposomes include water-in-oil-in-water emulsions as well as conventional liposomes (Strejan et al., J. Neuroimmunol. 1984; 7:27). Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the agent targeting the Apoptotic Sequence, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. Sterile injectable solutions can be prepared by incorporating the active agent that targets an Apoptotic Sequence in the required amount in an appropriate solvent with one or a combination of ingredients enumerated above, as required, followed by sterilization, e.g., microfiltration. Generally, dispersions are prepared by incorporating the agents targeted to Apoptotic Sequence into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. When the agent that targets Apoptotic Sequences of the present invention are administered as pharmaceuticals, for example to humans or animals, they can be given alone or as a pharmaceutical composition containing, for example, 0.01 to 99.5% (more preferably, 0.1 to 90%) of active ingredient in combination with a pharmaceutically acceptable carrier.

Actual dosage levels of the active agent that targets Apoptotic Sequences in the pharmaceutical compositions of the present invention may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A normal dosage, based on body weight and other pharmacological parameter will be known to a skilled practitioner (Sachdeva, Expert Opin Investig Drugs 1998; 7(11):1849-64).

A physician or veterinarian having ordinary skill in the art can readily determine and prescribe the effective amount of the pharmaceutical composition required. For example, the physician or veterinarian could start doses of the agent that targets Apoptotic Sequences of the invention employed in the pharmaceutical composition at levels lower than that required in achieving the desired therapeutic effect and gradually increasing the dosage until the desired effect is achieved. In general, a suitable daily dose of compositions of the invention will be that amount of the agent that targets an Apoptotic Sequence, which is the lowest dose effective to produce a therapeutic effect. Such an effective dose will generally depend upon the factors described above. If desired, the effective daily dose of a therapeutic composition may be administered as two, three, four, five, six or more sub-doses administered separately at appropriate intervals throughout the day, optionally, in unit dosage forms. While it is possible for a compound of the present invention to be administered alone, it may be administered as a pharmaceutical formulation (composition).

Therapeutic compositions can be administered with medical devices known in the art. For example, in one embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, discloses an implantable micro-infusion pump for dispensing medication at a controlled rate. U.S. Pat. No. 4,486,194, discloses a therapeutic device for administering medication through the skin. U.S. Pat. No. 4,447,233, discloses a medication infusion pump for delivering medication at a precise infusion rate. U.S. Pat. No. 4,447,224, discloses a variable flow implantable infusion apparatus for continuous drug delivery; U.S. Pat. No. 4,439,196, discloses an osmotic drug delivery system having multi-chamber compartments. U.S. Pat. No. 4,475,196, discloses an osmotic drug delivery system. These patents are incorporated herein by reference. Many other such implants, delivery systems, and modules are known to those skilled in the art.

A “therapeutically effective dosage” of a single or mixture of an agent that targets an Apoptotic Sequences may inhibit cancer cell growth and induced cell death in at least about 20%, by at least about 40%, by at least about 60%, or by at least about 80% of cancer cells present, relative to untreated subjects. The ability of an agent that targets an Apoptotic Sequence to inhibit cancer cell growth or induce cell death can be evaluated in an animal model system, such as those described in Examples 6 and 8-10, or other model systems known in the art that are predictive of efficacy in human conditions. Alternatively, the agent that targets an Apoptotic Sequence can be evaluated by examining its ability to inhibit or kill cancer cells using in vitro assays known to the skilled practitioner, including but not limited to the in vitro assays described in the Examples.

Agents Targeted to Apoptotic Sequences

An agent in a composition for therapeutic use may have a structure designed to achieve a well-known mechanism of activity including but not limited to a dsRNA-mediated interference (siRNA or RNAi), a catalytic RNA (ribozyme), a catalytic DNA, an aptazyme or aptamer-binding ribozyme, a regulatable ribozyme, a catalytic oligonucleotide, a nucleozyme, a DNAzyme, a RNA enzyme, a minizyme, a leadzyme, an oligozyme, or an antisense oligonucleotide. The agent targeted to an Apoptotic Sequence having properties comprising any of the types listed above may be between 6-10, 6-20, 6-30, 6-40, 6-50, 6-60, 6-70, 6-80, 6-90 and 6-100 nucleotides in length. Longer sequences can also be used.

In a non-limiting embodiment of the invention, the agent targeting an Apoptotic Sequence may be an antisense oligonucleotide sequence. The antisense sequence is complementary to at least a portion of the 5′ untranslated, 3′ untranslated or coding sequence of one or several Cancer Marker Sequences of a cancer cell's transcriptome as described above. An oligonucleotide sequence corresponding to the agent targeting an Apoptotic Sequence must be of sufficient length to specifically interact (hybridize) with the target Apoptotic Sequence but not so long that the oligonucleotide is unable to discriminate a single base difference. For example, for specificity the oligonucleotide is at least six nucleotides in length. Longer sequences can also be used. In a particular embodiment exemplified infra (Examples 6-10), the agent targeting Apoptotic Sequences may be 17 nucleotides in length. In another specific embodiment the agent targeting Apoptotic Sequences may have a DNA or RNA nucleotide sequence corresponding to SEQ ID NOS:1-7. The maximum length of the sequence will depend on maintaining its hybridization specificity, which depends in turn on the G-C content of the agent, melting temperature (Tₘ) and other factors, and can be readily determined by calculation or experiment e.g., stringent conditions for detecting hybridization of nucleic acid molecules as set forth in “Current Protocols in Molecular Biology”, Volume I, Ausubel et al., eds. John Wiley:New York N.Y., pp. 2.10.1-2.10.16, first published in 1989 but with annual updating) or by utilization of free software such Osprey (Nucleic Acids Research 32(17):e133) or EMBOSS (http://www.uk.embnet.org/Software/EMBOSS).

In another embodiment, the present invention provides for the design of inhibitory RNA sequences (RNAi or siRNA) based on Apoptotic Sequences. Design of siRNA molecules is well known in the art and established parameters for their design have been published (Elbashir, et al. EMBO J. 2001; 20: 6877-6888). For example a target sequence beginning with two AA dinucleotide sequences are preferred because siRNAs with 3′ overhanging UU dinucleotides are the most effective. It is recommended in siRNA design that G residues be avoided in the overhang because of the potential for the siRNA to be cleaved by RNase at single-stranded G residues. The siRNA designed on the basis of a target Apoptotic Sequence can be produced by methods, such as chemical synthesis, in vitro transcription, siRNA expression vectors, and PCR expression cassettes. Irrespective of which method one uses, the first critical step in designing a siRNA is to choose the siRNA target site. Since a target sequence including flanking nucleotides is available for each Apoptotic Sequence, design of a suitable siRNA molecule is well within the knowledge of a skilled practitioner. Oligonucleotide targeting agents which recognize small variations of a core Apoptotic Sequence target are provided for in the present invention. The design of a suitable family of siRNA molecules encompassing variant flanking sequences is well within the knowledge of a skilled practitioner. Thus, with knowledge of the target Apoptotic Sequence, the present invention provides for the design, synthesis, and therapeutic use of suitable siRNA molecules with will target Apoptotic Sequences in cancer cells.

In another embodiment, the present invention provides for the design of Ribozymes based on Apoptotic Sequences. Design and testing efficacy of ribozymes is well known in the art (Tanaka et al., Biosci Biotechnol Biochem. 2001; 65:1636-1644). It is known that a hammerhead ribozyme requires a 5′ UH 3′ sequence (where H can be A, C, or U) in the target RNA, a hairpin ribozyme requires a 5′ RYNGUC 3′ sequence (where R can be G or A; Y can be C or U; N represents any base), and the DNA-enzyme requires a 5′ RY 3′ sequence (where R can be G or A; Y can be C or U). Based on the foregoing design parameters and knowledge of the target Apoptotic Sequence, a skilled practitioner will be able to design an effective ribozyme either in hammerhead, hairpin or DNAzyme format. For testing the comparative activity of a given ribozyme, an RNA substrate which contains the common target sequence, i.e., an RNA containing an Apoptotic Sequence is used. Thus, with knowledge of the target Apoptotic Sequence, embodiments of the present invention provide for the design, synthesis, and therapeutic use of suitable ribozymes which target Apoptotic Sequences in cancer cells.

Design, Chemistry and Synthesis of an Agent Targeting an Apoptotic Sequence

An agent targeting an Apoptotic Sequence may be a DNA or a RNA molecule, or any modification or combination thereof. An agent targeting an Apoptotic Sequence may contain, inter-nucleotide linkages other than phosphodiester bonds, such as phosphorothioate, methylphosphonate, methylphosphodiester, phosphorodithioate, phosphoramidate, phosphotriester, or phosphate ester linkages (Uhlman et al., Chem. Rev. 1990; 90(4):544-584; Tidd, Anticancer Res. 1990; 10(5A):1169-1182), resulting in increased stability. Oligonucleotide stability may also be increased by incorporating 3′-deoxythymidine or 2′-substituted nucleotides (substituted with, e.g., alkyl groups) into the oligonucleotides during synthesis or by providing the oligonucleotides as phenylisourea derivatives, or by having other molecules, such as aminoacridine or poly-lysine, linked to the 3′ ends of the oligonucleotides (see, e.g., Tidd, 1990, supra). Modifications of the RNA and/or DNA nucleotides comprising the agent targeting Apoptotic Sequences of the invention may be present throughout the oligonucleotide or in selected regions of the oligonucleotide, e.g., the 5′ and/or 3′ ends.

The agent targeting Apoptotic Sequences can be made by any method known in the art, including standard chemical synthesis, ligation of constituent oligonucleotides, and transcription of DNA encoding the oligonucleotides, as described below.

Delivery Systems

Embodiments of the present invention provide for methods to increase the level of an agent targeting an Apoptotic Sequence in a target cell population or in a subject in need of treatment. Methods of delivery include but are not limited to physical methods mediated by chemical or biochemical formulations, physical force such as ballistic delivery, or by electrical methods such as electroporation. Delivery of an agent targeting an Apoptotic Sequence may be achieved without incorporation into an additional biological delivery agent such as a plasmid or virus vector.

Alternatively delivery into a cell or a subject is achieved by incorporating the agent targeting the Apoptotic Sequence into a biological vector. When incorporated into a biological vector, the agent will have extended persistence or half-life due to activity of a promoter which continually expresses the active form of the agent targeting an Apoptotic Sequence in a target cell population or a subject. The agent when incorporated into a biological vector may be delivered by a physical method as described above or by biologically mediated mechanisms such as receptor mediated cellular entry used by viruses.

Direct Delivery Methods

Where the expression level of an agent targeting an Apoptotic Sequence in a cell is to be increased by direct administration of the gene to a cell, the nucleic acid may be provided in a structure that facilitates uptake by a cell. For example, in alternative embodiments, the therapeutic nucleic acid may be provided in a liposome, microsphere or microbead (see infra).

Nanoparticle Compositions

In a particular embodiment the agent targeting an Apoptotic Sequence is an antisense oligonucleotide sequence incorporated into a gold nanoparticle-oligonucleotide complex (Au-NPOC) as described in Rosi et al., (Science 2006, 312:1027-1030). The antisense sequence of the Au-NPOC is complementary to at least a portion of the 5′ untranslated, 3′ untranslated or coding sequence of one or several cancer specific genes of a cancer cell's transcriptome as described infra. The antisense oligonucleotide sequence corresponding to the Apoptotic Sequence which is conjugated to the Au-NP may be at least six nucleotides in length, but can be up to about 100 nucleotides long. Longer sequences can also be used. In a specific embodiment the Apoptotic Sequence targeting agent is 17 nucleotides in length. In another specific embodiment the agent has either a DNA or RNA nucleotide sequence corresponding to SEQ ID NOS:1-7. In another specific embodiment the Apoptotic Sequence targeting agent is 17 nucleotides in length, either a DNA or RNA not containing any of SEQ ID NOS:1-7. In another embodiment the agent is an oligonucleotide conjugated to an Au-NP and may be composed of DNA, RNA, or any modifications or combinations thereof. The antisense sequence may be conjugated to the Au-NP by a tetra- or mono-thiol link. In another embodiment the antisense oligonucleotide strand density on an Au-NP may be between 20 to 180, between 45 to 120, or between 45-50 or 110-120 strands per particle depending on mono- or tetra-thiol linkage respectively. The strand density may be dependent on the coupling chemistry which includes but is not limited to mono- or tetra-thiol based conjugation.

An Au-NPOC incorporating an agent targeting an Apoptotic Sequence may readily enter a cell by direct uptake or may be mixed with commercially available lipofection compounds known to the art for delivery into cells (Rosi et al., Science, 2006; 312:1027-1030). In a specific embodiment, the agent when incorporated into an Au-NPOC has additional properties including but not limited to enhanced stability, lower susceptibility to nuclease degradation, non-toxicity to cells, deliverability at higher concentration, and deliverability with greater efficiency (higher percent transfection of cells in a population), compared to a corresponding non-Au-NPOC agent targeting an Apoptotic Sequence. Delivery by a ballistic or electrical method is also provided for by the invention.

Cell Penetrating Peptides

The plasma membrane of cells in a cell population or target tissue may be impermeable to hydrophilic compounds such as an oligonucleotide targeted to an Apoptotic Sequence. Embodiments of the present invention also provide for Apoptotic Sequence targeting-oligonucleotides to be modified so as to increase their ability to penetrate the target tissue by, e.g., coupling the oligonucleotides to a lipophilic compound (U.S. Pat. No. 5,386,023), a cell penetrating peptide or related delivery agent. In a specific embodiment the Apoptotic Sequence targeting agent is coupled to cell penetrating peptides (CPPs) or protein transduction domains (PTDs) using coupling chemistries known to a skilled practitioner. CPPs and PTDs have been characterized for their ability to translocate through the cellular plasma membrane (Takakura et al., Pharm Res. 1991; 7:339-346; Graslund et al., Genet Eng (NY) 2004; 26:19-31). When CPPs are linked to oligonucleotides, proteins, or nano-particles, they facilitate the transport of these entities across the cell membrane (Nori et al., Adv Drug Deliv Rev 2005; 57:609-636; Snyder et al., Pharm Res 2004; 21:389-393; Temsamani et al., Drug Discov Today 2004; 9:1012-1019). Non-limiting examples of CPPs include three of the most widely used CPPs: the Penetratin peptide (Antp), which is derived from the Drosophila transcription factor Antennapaedia (Derossi et al., J Biol Chem 1994; 269:10444-10450), the Tat peptide derived from the HIV-1 Tat protein (Weeks et al., J Biol Chem 1993; 268:5279-5284) and a hydrophobic peptide (MTS) derived from the Kaposi fibroblast growth factor signal peptide (Hawiger, Curr Opin Chem Biol 1999; 3:89-94).

In another specific non-limiting embodiment, an Apoptotic Sequence targeting agent is delivered into a population of cells in vitro or to a non-human animal or human subject by incorporation into “Vectosomes” (Normand et al., J Biol Chem 2001; 18:15042-15050). Vectosomes provide a means to deliver oligonucleotides to cells by mixing the oligonucleotide with a C-terminal fragment (“VP22.C1;” amino acid residues 159-301) of purified herpes simplex virus VP22 structural protein (Normand et al., supra). The VP22.C1 fragment interacts and forms electrostatic complexes with oligonucleotides, which are taken up more efficiently than lipofection based complexes (Normand et al., supra). Embodiments of the present invention also provide for nuclear targeting of an Apoptotic Sequence by a peptide-based gene delivery system, e.g., MPG, a fusion of the HIV-1 gp41 protein and nuclear localization signal of SV40 large T antigen (Simeoni et al., Nucl Acids Res 2003; 31:2717-2724).

A skilled artisan will know how to choose a suitable delivery agent described above depending on specific requirements of delivery in a therapy. In general, due to non-dependence on sequence-specific interactions between a delivery agent and oligonucleotide, mere mixing of components in appropriate ratios in the presence of a neutral carrier (e.g. Phosphate Buffered Saline (PBS) or Dublecco's Modified Eagle's Medium (DMEM)) suffices for complex formation. Addition of the preformed complex to a cell population by appropriate means such as injection, spraying or other means of application will result in uptake of the complex by target cells.

Delivery by Biological Vectors

Vectors and vector delivery systems may be biological agents that mediate delivery of an Apoptotic Sequence based therapeutic to a target cell population either in vitro or in a subject. A DNA vector construct comprising a sequence encoding a nucleic acid agent targeted to an Apoptotic Sequence is introduced into cells. The vector DNA construct includes additional functional components such as transcriptional regulatory elements, including a promoter element, an enhancer or UAS element, and a transcriptional terminator signal, for controlling the transcription of the Apoptotic Sequence in target cells. Mechanical methods, such as microinjection, ballistic DNA injection, liposome-mediated transfection, electroporation, or calcium phosphate precipitation, can be used to introduce such DNA constructs into target cells. Alternatively, one can use DNA delivery vectors to introduce the DNA into target cells.

Delivery Vectors

Suitable delivery vectors, which are often called expression vectors, include virus-based vectors and non-virus based DNA or RNA delivery systems. Examples of appropriate virus-based gene transfer vectors include, but are not limited to, pCEP4 and pREP4 vectors from Invitrogen, and, more generally, those derived from retroviruses, for example Moloney murine leukemia-virus based vectors such as LX, LNSX, LNCX or LXSN (Miller and Rosman, Biotechniques 1989; 7:980-989); lentiviruses, for example human immunodeficiency virus (“HIV”), feline leukemia virus (“FIV”) or equine infectious anemia virus (“EIAV”)-based vectors (Case et al., Proc. Natl. Acad. Sci. U.S.A. 1999; 96:22988-2993; Curran et al., Mol. Ther. 2000; 1:31-38; Olsen, Gene Ther. 1998; 5:1481-1487; U.S. Pat. Nos. 6,255,071 and 6,025,192); adenoviruses (Zhang, Cancer Gene Ther. 1999; 6:113-138; Connelly, Curr. Opin. Mol. Ther. 1999; 1:565-572; Stratford-Perricaudet, Human Gene Ther. 1990; 1:241-256; Rosenfeld, Science 1991; 252:431-434; Wang et al., Adv. Exp. Med. Biol. 1991; 309:61-66; Jaffe et al., Nat. Genet. 1992; 1:372-378; Quantin et al., Proc. Natl. Acad. Sci. U.S.A. 1992; 89:2581-2584; Rosenfeld et al., Cell 1992; 68:143-155; Mastrangeli et al., J. Clin. Invest. 1993, 91:225-234; Ragot et al., Nature 1993; 361:647-650; Hayaski et al., J. Biol. Chem. 1994; 269:23872-23875; Bett et al., Proc. Natl. Acad. Sci. U.S.A. 1994; 91:8802-8806), for example Ad5/CMV-based E1-deleted vectors (Li et al., Human Gene Ther. 1993; 4:403-409); adeno-associated viruses, for example pSub201-based AAV2-derived vectors (Walsh et al., Proc. Natl. Acad. Sci. U.S.A. 1992; 89:7257-7261); herpes simplex viruses, for example vectors based on HSV-1 (Geller and Freese, Proc. Natl. Acad. Sci. U.S.A. 1990; 87:1149-1153); baculoviruses, for example AcMNPV-based vectors (Boyce and Bucher, Proc. Natl. Acad. Sci. U.S.A. 1996; 93:2348-2352); SV40, for example SVluc (Strayer and Milano, Gene Ther. 1996; 3:581-587); Epstein-Barr viruses, for example EBV-based replicon vectors (Hambor et al., Proc. Natl. Acad. Sci. U.S.A. 1988; 85:4010-4014); alphaviruses, for example Semliki Forest virus- or Sindbis virus-based vectors (Polo et al., Proc. Natl. Acad. Sci. U.S.A. 1999; 96:4598-4603); vaccinia viruses, for example modified vaccinia virus (MVA)-based vectors (Sutter and Moss, Proc. Natl. Acad. Sci. U.S.A. 1992; 89:10847-10851), lentiviral microRNA-based systems (Stegmeier et al., Proc Natl Acad Sci USA, 2006; 102:13212-13217) or any other class of viruses that can efficiently transduce cells and that can accommodate the gene encoding an enzymatic or catalytic nucleic acid and sequences necessary and/or desirable for its expression.

In specific non-limiting embodiments of the invention, the promoter utilized to express the Apoptotic Sequence targeting agent may be selectively active in cancer cells; one example of such a promoter is the PEG-3 promoter, as described in International Patent Application No. PCT/US99/07199, Publication No. WO 99/49898 (published in English on Oct. 7, 1999); other non-limiting examples include the prostate specific antigen gene promoter (O'Keefe et al., Prostate 2000; 45:149-157), the kallikrein 2 gene promoter (Xie et al., Human Gene Ther. 2001; 12:549-561), the human alpha-fetoprotein gene promoter (Ido et al., Cancer Res. 1995; 55:3105-3109), the c-erbB-2 gene promoter (Takakuwa et al., Jpn. J. Cancer Res. 1997; 88:166-175), the human carcinoembryonic antigen gene promoter (Lan et al., Gastroenterol. 1996; 111:1241-1251), the gastrin-releasing peptide gene promoter (Inase et al., Int. J. Cancer 2000; 85:716-719). the human telomerase reverse transcriptase gene promoter (Pan and Koenman, Med. Hypotheses 1999; 53:130-135), the hexokinase II gene promoter (Katabi et al., Human Gene Ther. 1999; 10:155-164), the L-plastin gene promoter (Peng et al., Cancer Res. 2001; 61:4405-4413), the neuron-specific enolase gene promoter (Tanaka et al., Anticancer Res. 2001; 21:291-294), the midkine gene promoter (Adachi et al., Cancer Res. 2000; 60:4305-4310), the human mucin gene MUC1 promoter (Stackhouse et al., Cancer Gene Ther. 1999; 6:209-219), and the human mucin gene MUC4 promoter (Genbank Accession No. AF241535), which is particularly active in pancreatic cancer cells (Perrais et al., J Biol. Chem., 2001; 276(33):30923-33).

In an embodiment for expression of siRNA, ribozyme or antisense RNA molecules targeted to an Apoptotic Sequence expression is driven from a promoter for eukaryotic RNA polymerase I (pol I) or RNA polymerase III (pol III). Prokaryotic RNA polymerase promoters are also used, providing that the prokaryotic RNA polymerase enzyme is expressed in the appropriate cells (Elroy-Stein and Moss, Proc. Natl. Acad. Sci. USA, 1990; 87: 6743-7; Gao and Huang Nucleic Acids Res., 1993; 21:2867-72; Lieber et al., Methods Enzymol, 1993; 217, 47-66; Zhou et al., Mol. Cell. Biol., 1990; 10:4529 37). Embodiment of the present invention also provide for transcription units derived from genes encoding U6 small nuclear (snRNA), transfer RNA (tRNA), and adenovirus VA RNA which are particularly useful in generating high concentrations of desired RNA molecules such as ribozymes or siRNA in cells (Couture and Stinchcomb, Trends Genet. 1996; 12:510-515; Noonberg et al., Nucleic Acid Res., 1994, 22:2830; Noonberg et al., U.S. Pat. No. 5,624,803; Good et al., Gene Ther. 1997; 4, 45; Beigelman et al., International PCT Publication No. WO 96/18736). The above transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, Trends Genet. 1996; 12:510-515).

Alternatively, if it is desired that the DNA construct be stably retained by the cells, the DNA construct can be supplied on a plasmid and maintained as a separate element using an episomal vector or integrated into the genome of the cells using an integrating vector such as a plasmid containing a selectable marker and additional elements with promote genomic integration or an integrating virus such as a retrovirus (see Couture and Stinchcomb, supra).

In an alternative embodiment of the present invention genes that frequently contained apoptotic sequences in patients with cancer may be targeted by Apoptotic Sequence targeting agents described above but tailored to the specific sequence in question. The target sequences are identified by computational analysis to compare selected apoptotic sequences with databases of genes to determine genes that frequently contained apoptotic sequences in patients with cancer. These genes may serve as a target for inducing apoptosis in cancer cells even though most or all are not oncogenes. These genes may only be expressed in cancer cells, or treatment may focus on variations of these genes having only apoptotic sequences for targeting. Examples of such genes are shown in Table 6.

### EXAMPLES

The present invention may be better understood by reference to the following examples, which are provided by way of explanation and not by way of limitation.

### Example 1

### Apoptotic Sequence Targeting Agents

Examples of targeting agents to Apoptotic Sequences, in antisense oligonucleotide form, according to embodiments of the present invention are shown in Table 1. Each ID number indicated in the table is used when referring to that Apoptotic Sequence throughout this specification, for example in the experiments described in the Figures. Table 5 shows both strands of an identified Apoptotic Sequence while Table 1 shows the actual targeting agent corresponding to the respective Apoptotic Sequence. Although Apoptotic Sequence targeting agents need not all be a specific length, the agents of Table 1 are all 17 base pairs in length, allowing specificity, but facilitating function.

TABLE 1
Apoptotic Sequence Targeting Agents

| | |
|---|---|
| ID | Apoptotic Sequence Targeting Agent |

| | |
|---|---|
| 5 | AAGGGGGTTCCTTGGGC |
| | (SEQ ID NO: 1) |
| 8 | CCTGAGCAAACCTGAGC |
| | (SEQ ID NO: 6) |
| 9 | GGCCTGCCAGAAGCACA |
| | (SEQ ID NO: 2) |
| 11 | CGCATGCGTGGCCACCA |
| | (SEQ ID NO: 7) |
| 14 | GCCGATTAACACCAGCC |
| | (SEQ ID NO: 3) |
| 60 | CGATTAACCACCGGCCT |
| | (SEQ ID NO: 4) |
| 66 | TTGAACCCTAGGCATGT |
| | (SEQ ID NO: 5) |

In a particular embodiment, a nucleic acid or nucleic acids, such as DNA specifically targeting one or more Apoptotic Sequences are provided for, and used to induce death in a cancer cell. An Apoptotic Sequence nucleic acid targeting agent is provided in a physiologically acceptable carrier, such as PBS or CSF solution, to form a treatment formulation. This treatment formulation is administered to the cancer cell through the blood, spinal fluid, or by intratumoral injection. A normal dosage, based on body weight, of each Apoptotic Sequence targeting agent DNA from Table 1 has been administered to several mice, and 10 times the normal dosage has been administered to 5 mice. Normal DNA administration was 5 mg per 1 kg of body weight, mixed in a ratio of 10 mg DNA per 1 ml PBS or CSF. Normal DNA administration for humans may be between 5 mg and 25 mg per kg of body weight.

### Example 2

### Multi-Gene Aspect

Many genes may be associated with each Apoptotic Sequence. Sometimes, hundreds of mRNA transcripts may contain a single Apoptotic Sequence. The common appearance of these Apoptotic Sequences, which may be cancerous mutations, in many genes is not presently understood. However, it is this commonality in multiple genes that may facilitate the cancer cell-differentiating ability of Apoptotic Sequences and their cancer cell death inducing ability.

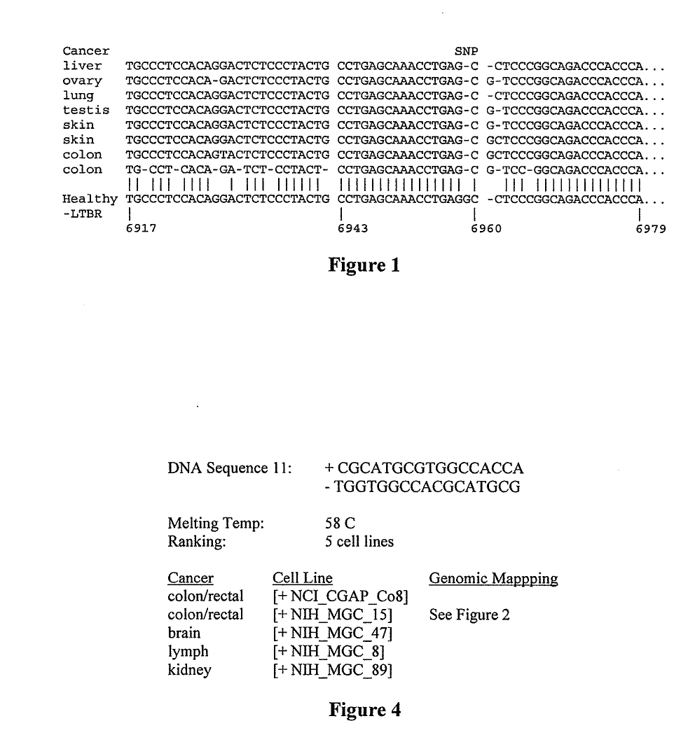

While most candidate Apoptotic Sequences are located in genes with no currently known relevance to cancer, some are located in genes known to be important in cancer. These sequences often manifest themselves as SNPs, cryptic splicing and other genetic defects. For example, FIG. 1 illustrates an Apoptotic Sequence found in the Lymphotoxin Beta Receptor (LTBR) gene.

FIG. 1 shows that the same point mutation occurs in the same gene in different subjects with different types of cancer. Specifically, FIG. 1 shows a portion of an alignment between LTBR mRNA from eight different cancer cell lines and six different cancer types, mapped to the corresponding healthy LTBR mRNA. As the figure shows, the eight cancer LTBRs (SEQ ID NOS: 133-140) vary slightly between each other and the healthy LTBR (SEQ ID NO: 141). However at location 6959 bp, the cancer LTBRs vary identically, each missing a guanine (G) and yielding the same Apoptotic Sequence target, CCTGAGCAAACCTGAGC (SEQ ID NO:6).

FIG. 2 shows that the same Apoptotic Sequence can occur in common regions in different mutations, different genes, in different subjects, and different types of cancer. Specifically, FIG. 2 shows a portion of an alignment between mRNA from four different cancer cell lines and four different cancer types (SEQ ID NOS:142-158), aligned with the corresponding healthy mRNA from different genes. The overall alignments vary from gene to gene, but each has a common region yielding the Apoptotic Sequence, CGCATGCGTGGCCACCA (SEQ ID NO:7).

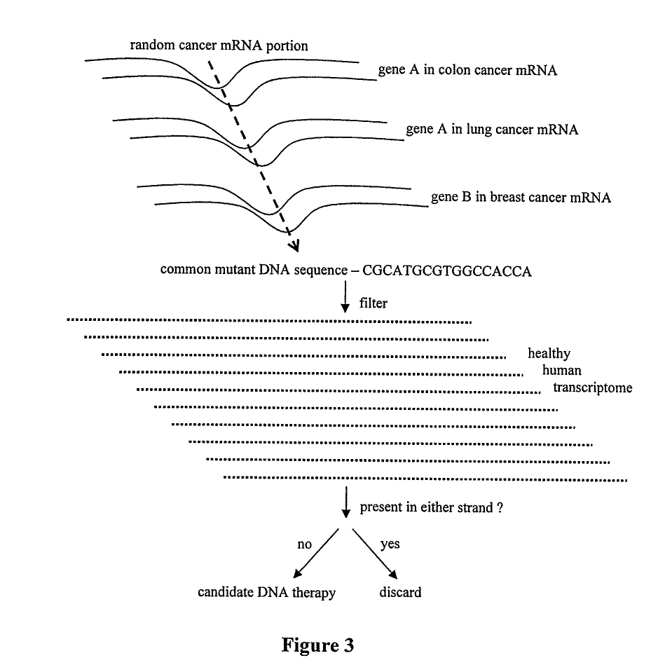

The Apoptotic Sequences shown in FIGS. 1, 2 and 3 are not dependent on any common functionality among the genes in which they appear, or in the tissues in which these genes are expressed. Further, none of the sequences has been found in the healthy human transcriptome. Therefore the presence of these sequences in any mRNA transcript, not just those from genes shown in the figures, may be an indicator of cancer's presence in the host cell.

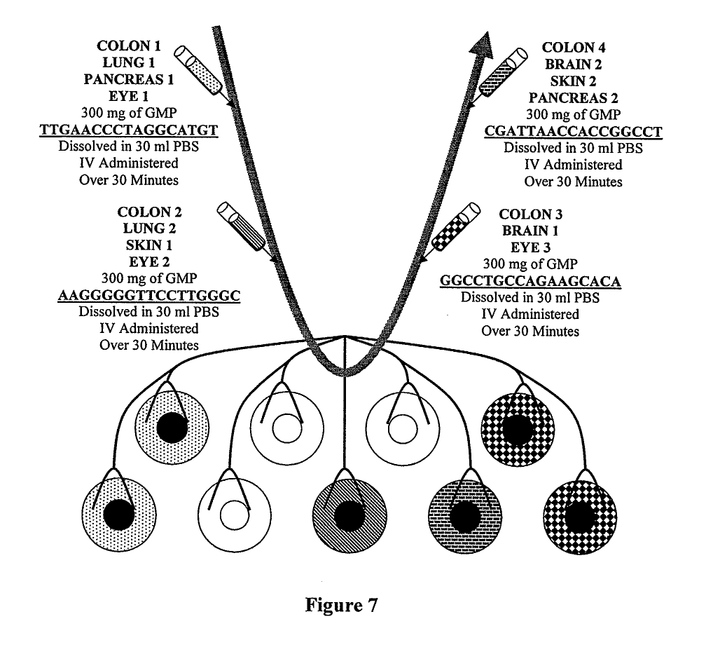

Apoptotic Sequences may be common to many genes and many cancers. This does not mean that they will exist in every cancer cell line or cancer subject. Therefore it is desirous to know which Apoptotic Sequences correspond to a subject's individual cancer. Then the sequences can be used to make an appropriate Apoptotic Sequence targeting agent treatment formulation. This is illustrated in FIG. 7 where a single cancer may require multiple different nucleic acids to eradicate all the cancer cells. The figure also shows the overlap in the Apoptotic Sequences between cancer types. So one treatment formulation may be effective against many types of cancer, but no two cancer subjects should be presumed as having the same cancer mutations. This flexibility gives the Apoptotic Sequences' treatment formulations superiority over the rigid targeting of current chemotherapies.

### Example 3

### Multi Gene or Single Gene Apoptotic Sequences

Table 2 shows further information for the Apoptotic Sequences of Table 1. In particular, it provides their multi-gene or single gene mapping characterizations. In the case of single gene Apoptotic Sequences, the recognized National Institutes of Health (NIH) gene names are provided. Also provided and shown in parentheses are common alias names given to the mapped gene, and genes that are similar to the mapped gene and contain the Apoptotic Sequence as well. In the latter case, most of these genes are predicted and have yet to be characterized by NIH.

TABLE 2
Gene Mapping of Apoptotic Sequences

| | | | Subject | | (Alias |
|---|---|---|---|---|---|
| | Phosphorothioated | | R colon | | Names) & |
| | Apoptotic Sequence | Healthy | cancer | Target Gene | Similar |
| ID | Targeting Agent | cells | cells | Characterization | Genes |
| 5 | AAGGGGGTTCCTTGGGC | 10% | 82% | multi-gene | |
| | (SEQ ID NO: 1) | | | | |
| 9 | GGCCTGCCAGAAGCACA | 9% | 70% | GNB2L1 | (RACK1) |
| | (SEQ ID NO: 2) | | | | |
| 14 | GCCGATTAACACCAGCC | 15% | 72% | multi-gene | |
| | (SEQ ID NO: 3) | | | | |
| 60 | CGATTAACCACCGGCCT | 12% | 73% | multi-gene | |
| | (SEQ ID NO: 4) | | | | |
| 66 | TTGAACCCTAGGCATGT | 8% | 83% | EEF1A1 | EEF1A2 |
| | (SEQ ID NO: 5) | | | | LOC441032 |
| | | | | | LOC440595 |
| | | | | | LOC442709 |
| | | | | | LOC442332 |

### Example 4

### In Vitro Cancer Cell Detection Tests

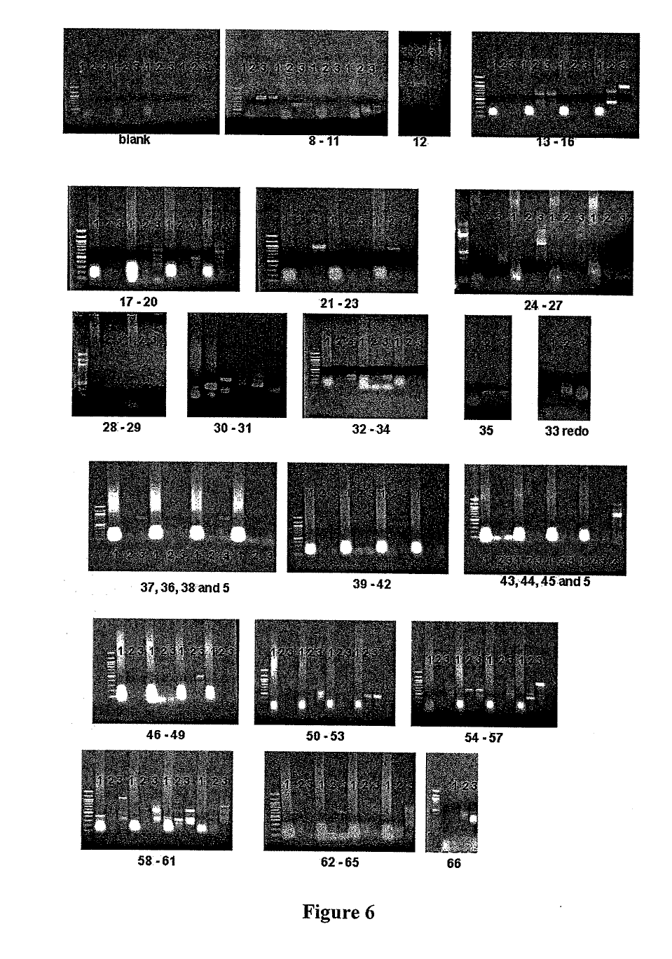

The cancer cell differentiation abilities of the candidate Apoptotic Sequences from Table 5 were tested for their presence in cancer cells and absence in healthy cells. The general method of this testing is shown in FIG. 5. Testing was conducted using an excised 9 mm tumor and a 20 ml blood sample, taken at different times, from Subject R and a 20 ml blood sample from Subject H. Subject R was a female human patient with metastasized colon cancer. Subject H was a male human patient also with metastasized colon cancer. The multi-gene, one-to-many aspect of Apoptotic Sequences yields sensitivity sufficient to allow detection of metastasized cancer cells in blood samples as well as biopsy tissues, as shown in FIG. 6. The healthy control sample used in the tests must be carefully selected because of this sensitivity. It is possible for cancer to be detected in what is otherwise believed to be healthy cells. Therefore, a healthy control sample from tissue not normally associated with cancer, like vascular walls, is used.

Table 3A shows the results of single priming RT-PCR using the primers with the Apoptotic Sequences from Table 5, i.e. anti-sense oligo primers synthesized from sixty-six cancer mutations isolated by the method described supra. Tests were performed on RNA from a clinical human cancer sample (RNA isolated from freshly excised and cultured tissue of “Subject R”, a colon cancer patient), and a vascular wall healthy control sample (vascular endothelial cell line). A filled-in column in Table 3A indicates a sequence's presence and an empty column indicates a sequence's absence. Those sequences found in the healthy control sample were discarded from the candidate Apoptotic Sequence pool, while the others are available for subsequent cell death tests.

TABLE 3A
Cancer-Unique Mutations in a Single Tumor: Patient R

| | 01 | 02 | 03 | 04 | 05 | 06 | 07 | 08 | 09 | 10 | 11 | 12 | 13 | 14 | 15 | 16 | 17 | 18 |
|---|---|---|---|---|---|---|---|---|---|---|---|---|---|---|---|---|---|---|
| Healthy mRNA | ▪ | | | | | | | | | | | | | | | | | |
| R Tumor mRNA | ▪ | | | | ▪ | | | ▪ | ▪ | | ▪ | ▪ | | ▪ | | ▪ | | |
| | 19 | 20 | 21 | 22 | 23 | 24 | 25 | 26 | 27 | 28 | 29 | 30 | 31 | 32 | 33 | 34 | 35 | 36 |
| Healthy mRNA | | | | | | | | | | | | | | | | | | |
| R Tumor mRNA | | ▪ | | | ▪ | | | ▪ | | | | ▪ | ▪ | | ▪ | | ▪ | |

| | 37 | 38 | 39 | 40 | 41 | 42 | 43 | 44 | 45 | 46 | 47 | 48 | 49 | 50 | 51 |
|---|---|---|---|---|---|---|---|---|---|---|---|---|---|---|---|
| Healthy mRNA | | | | | | | | | | | | | | | |
| R Tumor mRNA | | | | | | | | | | | | | | | |
| | 52 | 53 | 54 | 55 | 56 | 57 | 58 | 59 | 60 | 61 | 62 | 63 | 64 | 65 | 66 |
| Healthy mRNA | | | | | | | ▪ | | | | | | | | |
| R Tumor mRNA | | ▪ | | ▪ | | ▪ | ▪ | | ▪ | | | | | | ▪ |

The number of cancer-unique mutations found in Patient R's tumor led to the hypothesis that all of the tumor cells do not possess the same mutations, and more broadly all the tumors in Patient R (five at the time of testing) did not possess the same mutations. It is possible that due to their multiple gene nature, primers synthesized from the mutations possessed a more robust detection capacity than single gene primers because detection is not dependent on the expression characteristics of only a single gene. Thus, due to possible presence of the Apoptotic Sequence in more than a single gene, detection sensitivity may be enhanced permitting detection of metastasized cancer cell traffic derived from Patient R's multiple tumors using only a blood sample.

TABLE 3B
Cancer-Unique Mutations in a Tumor versus Bloodstream (Patient R)

| | 01 | 02 | 03 | 04 | 05 | 06 | 07 | 08 | 09 | 10 | 11 | 12 | 13 | 14 | 15 | 16 | 17 | 18 |
|---|---|---|---|---|---|---|---|---|---|---|---|---|---|---|---|---|---|---|
| Healthy mRNA | ▪ | | | | | | | | | | | | | | | | | |
| R Tumor mRNA | ▪ | | | | ▪ | | | ▪ | ▪ | | ▪ | ▪ | | ▪ | | ▪ | | |
| R Blood mRNA | ▪ | | | | ▪ | | | ▪ | ▪ | | ▪ | ▪ | ▪ | ▪ | | ▪ | ▪ | |
| | 19 | 20 | 21 | 22 | 23 | 24 | 25 | 26 | 27 | 28 | 29 | 30 | 31 | 32 | 33 | 34 | 35 | 36 |
| Healthy mRNA | | | | | | | | | | | | | | | | | | |
| R Tumor mRNA | | ▪ | | | ▪ | | | ▪ | | | | ▪ | ▪ | | ▪ | | ▪ | |
| R Blood mRNA | | ▪ | | ▪ | | | ▪ | ▪ | | ▪ | | ▪ | ▪ | ▪ | ▪ | | ▪ | |

| | 37 | 38 | 39 | 40 | 41 | 42 | 43 | 44 | 45 | 46 | 47 | 48 | 49 | 50 | 51 |
|---|---|---|---|---|---|---|---|---|---|---|---|---|---|---|---|
| Healthy mRNA | | | | | | | | | | | | | | | |
| R Tumor mRNA | | | | | | | | | | | | | | | |
| R Blood mRNA | ▪ | | | | | ▪ | | | | | | ▪ | | | ▪ |
| | 52 | 53 | 54 | 55 | 56 | 57 | 58 | 59 | 60 | 61 | 62 | 63 | 64 | 65 | 66 |
| Healthy mRNA | | | | | | | ▪ | | | | | | | | |
| R Tumor mRNA | | ▪ | | ▪ | | ▪ | ▪ | | ▪ | | | | | | ▪ |
| R Blood mRNA | | ▪ | | ▪ | ▪ | ▪ | ▪ | ▪ | ▪ | ▪ | ▪ | | ▪ | | ▪ |

Table 3B lists the results of the sixty-six mutations from Table 3A, expanded to include the mRNA from 20 ml of Patient R's blood sample. The table shows that the mutations found in Patient R's single tumor are roughly a subset of the mutations found in the patient's bloodstream. Table 3B implies that different tumors within the same patient possess different mutations. It follows that different cancer patients possess different mutations. A 20 ml blood sample was taken from another colon cancer patient, Patient H, and the same RT-PCR tests were run. Table 3C shows the comparative results between Patient R and Patient H.

TABLE 3C
Cancer-Unique Mutations in Two Bloodstreams (Patient R & Patient H)

| | 01 | 02 | 03 | 04 | 05 | 06 | 07 | 08 | 09 | 10 | 11 | 12 | 13 | 14 | 15 | 16 | 17 | 18 |
|---|---|---|---|---|---|---|---|---|---|---|---|---|---|---|---|---|---|---|
| Healthy mRNA | ▪ | | | | | | | | | | | | | | | | | |
| R Blood mRNA | ▪ | | | | ▪ | | | ▪ | ▪ | | ▪ | ▪ | ▪ | ▪ | | ▪ | ▪ | |
| H Blood mRNA | ▪ | | | | ▪ | | | ▪ | ▪ | | ▪ | | | ▪ | | ▪ | | |
| | 19 | 20 | 21 | 22 | 23 | 24 | 25 | 26 | 27 | 28 | 29 | 30 | 31 | 32 | 33 | 34 | 35 | 36 |
| Healthy mRNA | | | | | | | | | | | | | | | | | | |
| R Blood mRNA | | ▪ | | ▪ | | | ▪ | ▪ | | ▪ | | ▪ | ▪ | ▪ | ▪ | | ▪ | |
| H Blood mRNA | ▪ | | ▪ | | | | ▪ | ▪ | | | | ▪ | | ▪ | ▪ | | ▪ | |

| | 37 | 38 | 39 | 40 | 41 | 42 | 43 | 44 | 45 | 46 | 47 | 48 | 49 | 50 | 51 |
|---|---|---|---|---|---|---|---|---|---|---|---|---|---|---|---|
| Healthy mRNA | | | | | | | | | | | | | | | |
| R Blood mRNA | ▪ | | | | | ▪ | | | | | | ▪ | | | ▪ |
| H Blood mRNA | ▪ | ▪ | | | | | | | | | | ▪ | | | ▪ |
| | 52 | 53 | 54 | 55 | 56 | 57 | 58 | 59 | 60 | 61 | 62 | 63 | 64 | 65 | 66 |
| Healthy mRNA | | | | | | | ▪ | | | | | | | | |
| R Blood mRNA | | ▪ | | ▪ | ▪ | ▪ | ▪ | ▪ | ▪ | ▪ | ▪ | | ▪ | | ▪ |
| H Blood mRNA | | ▪ | ▪ | ▪ | ▪ | ▪ | | ▪ | ▪ | ▪ | | ▪ | | ▪ | ▪ |

Table 3C shows that nearly two-thirds of the sixty-six cancer-unique mutations (Cancer Marker Sequences) were found in the two patients' bloodstream. This confirmed the robust detection capability of primers synthesized with the mutation nucleotides. The table also confirmed that cancer mutations vary from patient to patient, being both common and unique. This implies that no single cancer treatment can address each patient, or perhaps even each tumor in a single patient.

### Example 5

### In Vitro Cancer Cell Death Tests

Candidate Apoptotic Sequences were identified from the Cancer Marker Sequences above, which differentiated between healthy and cancer cells, by testing for the capacity to kill the cancer cells. A sequence's ability to differentiate between healthy and cancer cells does not necessarily mean it can kill the cancer cells. Although most of the candidate Apoptotic Sequences may be used to partially or completely down-regulate expression of many genes in cancer cells, this may not be sufficient to kill the cells. A candidate Apoptotic Sequence when targeted, should necessarily possess both the ability to differentiate cancer from normal cells, and kill the cancer cells.

Anti-sense phosphorothioated DNA (S-oligos) were synthesized from the eighteen cancer-unique mutations found in both Patient R's tumor and bloodstream from Table 3B (#1 & #58 were not used). The S-oligos were mixed in a buffered treatment formulation and individually exposed to 20,000 cells from Patient R's tumor over a several day period. This was followed by MTT cell proliferation assays. Six of the S-oligos caused significant tumor cell death, as shown in Table 3D.

Procedure for Transfection of Cells Using Maxfect™

Cell suspensions from each cell line were prepared at a density of 2×10⁶ cells/16 ml medium. To each well of a 96-well plate, 160 μl of the cell suspension (˜20,000 cells) was added. Cells were allowed to grow for 24 h at 37° C. in an atmosphere of 95% air/5% CO2 and 100% humidity. After 24 h, cells had attached to the plate and were ready for transfection. A stock oligo solution was prepared in sterile water and an aliquot of the stock was diluted with sterile serum-free medium without antibiotics in siliconized tubes and the ratio of oligo solution to medium kept constant at 1/20.

The Maxfect™ lipid, a transfection reagent, was diluted by adding 1 μl of Maxfect™ lipid to 20 μl of serum-free medium without antibiotics in siliconized tubes. The oligo solution prepared from the prior steps was added directly to the diluted Maxfect™ solution. The two solutions were mixed by tapping the tubes or by repeatedly pipetting the liquid followed by incubation at room temperature for 20 min.

The wells containing the cells from the first plating step were washed with serum- and antibiotic-free medium. To the washed cells in each well, 60 μl of serum and antibiotic-free medium was added. 40 μl of the oligo/Maxfect™ complex from the prior steps was added to each well to give a total volume of 100 μl. Cells were incubated at 37° C. for 6 h in a tissue culture incubator supplied with 5% CO2. At the end of 6 h incubation, 100 μl of medium containing 2 times of normal concentrations of serum and antibiotics (2× medium) was added to each well. The cells were incubated for an additional 12-24 h under normal cell culture conditions. At the end of incubation, the medium in each well was aspirated and replaced with fresh 1× medium containing the normal additives for cell culture. The cells were incubated for additional 96 h.

MTT Assay

At the end of 96 h, an MTT assay (Promega Corporation, Madison, Wis.) was performed. The MTT assay was conducted by adding 15 μL of tetrazolium dye solution to each well and continuing incubation of cells for an additional 4 h. During this 4-h incubation period, viable cells converted the dye component of the tetrazolium salt to a formazan product, which is blue. After 4 hours, 100 μl of Solubilization/Stop solution was added to each well. The plate was kept at room temperature overnight, and the blue color of the product was measured at 575 nm on an ELISA plate reader. The absorbance obtained for the cells treated with oligos relative to that obtained for the control cells gave the % of inhibition on cell growth.

TABLE 3D
MTT Cell Proliferation Assay of S-oligo Effect on 20,000 Tumor Cells (Patient R)

| | | | | | | | | | | | | | | | | | |
|---|---|---|---|---|---|---|---|---|---|---|---|---|---|---|---|---|---|
| 05 | 08 | 09 | 11 | 12 | 14 | 16 | 20 | 26 | 30 | 31 | 33 | 35 | 53 | 55 | 57 | 60 | 66 |

% Inhibition

| | | | | | | |
|---|---|---|---|---|---|---|
| R | 82 | 70 | 75 | 72 | 73 | 80 |
| Tumor | | | | | | |
| Cells | | | | | | |

Table 3D demonstrates that the reduction in expression of the set of genes corresponding to mutations 05, 09, 11, 14, 60, and 66 cause cell death. The death may be due to the reduction of expression of a single gene or a combination of genes in each set of genes whose expression may be affected by an agent targeting a specific Apoptotic Sequence. The percent inhibitions in the table may correspond to the same or different subsets of tumor cells each possessing a specific type of mutation response to a particular Apoptotic Sequence within the population of 20,000 tumor cells tested. Since the six S-oligos represent six different mutations, and may cause reduction of six different set of genes, an overlapping portion of the 20,000 tumor cells may be affected by the activity of each Apoptotic Sequence. This implies that, when combined or sequentially administered, the S-oligos are capable of causing apoptosis in 100% of the tumor cells.

The six S-oligos causing cell death in Table 3D were contacted to three known colon cancer cells lines and tested for their effectiveness against additional colon cancers. These results are shown in Table 3E.

TABLE 3E
Assay of S-oligo Effect on Colon Cancer
Cells by MTT Cell Proliferation Assay

| | | % Inhibition | | | | | |
|---|---|---|---|---|---|---|---|
| Cell Line | Cancer | 05 | 09 | 11 | 14 | 60 | 66 |
| Patient R | colon | 82.00 | 70.00 | 75.00 | 72.00 | 73.00 | 83.00 |
| Tumor | | | | | | | |
| DLD-1 | colon | 62.6 | 48.51 | 65.40 | 54.37 | 49.14 | 45.78 |
| HT-29 | colon | 52.68 | 59.48 | 43.39 | 64.15 | 24.92 | 0.00 |
| LoVo | colon | 100.00 | 84.93 | 96.19 | 100.00 | 29.25 | 8.42 |

In addition to the experiments described in Table 3E, the six oligos were exposed to known cell lines of various cancers to study their effectiveness against cancer in general and to repeat the assertion that they do not interfere with normal cells. An optimized number of cells for each cell type tested was determined (Table 3F) prior to performing the actual tests so as to ensure efficient transfection, and growth condition for each different type of human cancer derived cell line used in MTT proliferation tests (Table G). The cell number seeded in tissue culture wells for each cell line had to be empirically determined so as to ensure optimized MTT proliferation assays while testing the effect on cancer cell growth of the oligonucleotides.

The six oligonucleotides were evaluated in 16 human cancer cell lines and one normal endothelial cell line for the study. The endothelial cell line was normal primary microvascular endothelial cells (HMVEC). The HMVEC cells, medium and growth factors for culturing HMVEC cells were supplied by Cambrex Bioproducts (Walkersville, Md.). A test was also run on the normal cell line to determine the inhibition effect of the Maxfect (transfection reagent) alone without any oligos. The Maxfect alone showed an inhibition of 5.0. The results of these tests are shown below in Tables 3G-L.

From the results seen in Tables 3G-L it appears that any one of the Apoptotic Sequences targeting agents tested is capable of causing growth inhibitory activity in many types of cancer cells including breast, ovarian, colon, lung or brain cancer. This observation further emphasizes the likelihood that an agent to a single Apoptotic Sequence may be targeting multiple cancer specific genes. As a result of this broad specificity the targetability of a given Apoptotic Sequence is not confined to a single cancer type.

TABLE 3F
Summary of cell numbers used for cell lines tested

| Cell | | Cell |
|---|---|---|
| Line | Cell Type | Number/Well |
| MCF-7 | Breast cancer | 8 × 10³ |
| MDAMB231 | Breast cancer | 8 × 10³ |
| MDAMB468 | Breast cancer | 8 × 10³ |
| ZR-75-1 | Breast cancer | 10 × 10³ |
| A549 | Lung cancer | 8 × 10³ |
| Calu-1 | Lung cancer | 8 × 10³ |
| NCI-H460 | Lung cancer | 10 × 10³ |
| A427 | Lung cancer | 12 × 10³ |
| U87 | Brain cancer, glioblastoma | 10 × 10³ |
| U118 | Brain cancer, glioblastoma | 10 × 10³ |
| Daoy | Brain cancer, medulloblastoma | 8 × 10³ |
| DLD-1 | Colon cancer | 8 × 10³ |
| HT-29 | Colon cancer | 8 × 10³ |
| LoVo | Colon cancer | 8 × 10³ |
| SKOV-3 | Ovarian cancer | 12 × 10³ |
| OVCAR-3 | Ovarian cancer | 20 × 10³ |
| HMVEC | Human endothelial microvascular cells | 20 × 10³ |

TABLE 3G
MTT Cell Proliferation Assay to Determine the Growth Inhibitory
Effect of S-oligos on Cancer Cells and Normal Cells

| | | % Inhibition | | | | | |
|---|---|---|---|---|---|---|---|
| Cell Line | Cancer | C 5 | C 9 | C 11 | C 14 | C 60 | C 66 |
| Normal | N/A | 0.0- | 14.0- | 11.0- | 6.0- | 0.0- | 0.0- |
| | | 16.0 | 15.0 | 17.0 | 16.0 | 16.0 | 8.0 |
| Patient R | colon | 82.00 | 70.00 | 75.00 | 72.00 | 73.00 | 83.00 |
| Tumor | | | | | | | |
| DLD-1 | colon | 62.60 | 48.51 | 65.40 | 54.37 | 49.14 | 45.78 |
| HT-29 | colon | 52.68 | 59.48 | 43.39 | 64.15 | 24.92 | 0.00 |
| LoVo | colon | 100.00 | 84.93 | 96.19 | 100.00 | 29.25 | 8.42 |
| Calu-1 | lung | 55.77 | 42.17 | 52.07 | 65.11 | 52.46 | 57.26 |
| A549 | lung | 95.48 | 81.76 | 96.96 | 90.67 | 76.62 | 80.22 |
| A427 | lung | 42.15 | 51.26 | 39.03 | 52.51 | 36.24 | 45.50 |
| NCIH460 | lung | 66.22 | 64.79 | 66.11 | 76.45 | 67.50 | 66.21 |
| U118 | brain | 40.54 | 50.57 | 44.01 | 47.36 | 49.08 | 49.02 |
| Daoy | brain | 86.03 | 41.16 | 84.22 | 80.66 | 42.65 | 67.03 |
| U87 | brain | 81.52 | 85.96 | 85.77 | 91.41 | 83.42 | 85.92 |
| OVCAR-3 | ovary | 62.39 | 28.09 | 27.73 | 34.29 | 6.20 | 20.28 |
| SKOV-3 | ovary | 13.92 | 16.80 | 17.86 | 18.02 | 16.75 | 25.57 |
| MCF7 | breast | 69.4 | 40.00 | 66.80 | 49.70 | 44.50 | 34.90 |
| MDAMB-231 | breast | 52.77 | 51.65 | 41.82 | 44.40 | 43.84 | 35.05 |
| ZR-75-1 | breast | 81.78 | 80.53 | 92.00 | 99.96 | 86.56 | 70.64 |
| MDAMB-468 | breast | 73.21 | 72.47 | 82.63 | 76.03 | 72.96 | 71.16 |

TABLE 3H
Effect of S-oligos on proliferation of human
brain cancer and endothelial cells

| Cell Line | | U87 | U118 | DAOY | HMVEC* |
|---|---|---|---|---|---|
| Oligo | Dose (μM) | % Inhibition | | | |
| C5 | 1 | 71.09^{a} | 17.92^{a} | 80.87^{a} | 16.00 |
| | 0.25 | 81.52^{a} | 36.41^{a} | 86.03^{a} | −2.00 |
| | 0.0625 | 78.41^{a} | 40.54^{a} | 51.37^{a} | 2.00 |
| C9 | 1 | 78.73^{a} | 50.57^{a} | 37.79^{a} | 14.00 |
| | 0.25 | 85.96^{a} | 45.93^{a} | 41.16^{a} | 14.00 |
| | 0.0625 | 83.87^{a} | 44.79^{a} | 36.09^{a} | 15.00 |
| C11 | 1 | 84.84^{a} | 43.35^{a} | 81.14^{a} | 11.00 |
| | 0.25 | 85.65^{a} | 42.24^{a} | 84.22^{a} | 17.00 |
| | 0.0625 | 85.77^{a} | 44.01^{a} | 70.9^{a} | 17.00 |
| C14 | 1 | 81.55^{a} | 47.36^{a} | 80.66^{a} | 16.00 |
| | 0.25 | 91.41^{a} | 42.48^{a} | 67.40^{a} | 9.00 |
| | 0.0625 | 86.02^{a} | 38.95^{a} | 53.92^{a} | 6.00 |
| C60 | 1 | 78.15^{a} | 49.08^{a} | 38.50^{a} | 15.00 |
| | 0.25 | 83.42^{a} | 43.36^{a} | 32.65^{a} | 16.00 |
| | 0.0625 | 83.39^{a} | 34.60^{a} | 42.65^{a} | −3.00 |
| C66 | 1 | 73.81^{a} | 49.02^{a} | 52.49^{a} | 8.00 |
| | 0.25 | 85.92^{a} | 39.59^{a} | 67.03^{a} | −3.00 |
| | 0.0625 | 78.23^{a} | 29.67^{a} | 36.02^{a} | 4.00 |

*Normal endothelial cells
^{a}Inhibitory effect is statistically significant (P < 0.001)

TABLE 3I
Effect of S-oligos on proliferation of human breast cancer cells

| Cell Line | | MDAMB-231 | ZR-75-1 | MDAMB-468 | MCF-7 |
|---|---|---|---|---|---|
| Oligo | Dose (μM) | % Inhibition | | | |
| C5 | 1 | 41.06^{a} | 81.78^{b} | 73.20^{a} | 67.60^{f} |
| | 0.25 | 52.77^{a} | 80.95^{b} | 51.71^{b} | 69.40 |
| | 0.0625 | 44.98^{a} | 46.36 | 64.91^{a} | 41.10 |
| C9 | 1 | 41.71^{a} | 65.45 | 72.40^{a} | 19.30 |
| | 0.25 | 39.55^{a} | 75.25^{e} | 68.40^{a} | 40.00^{c} |
| | 0.0625 | 51.65^{a} | 80.53^{c} | 52.77^{b} | 35.20^{c} |
| C11 | 1 | 34.26^{a} | 92.00^{c} | 82.63^{a} | 61.70^{a} |
| | 0.25 | 34.07^{a} | 89.74^{b} | 73.70^{a} | 66.80^{a} |
| | 0.0625 | 41.82^{a} | 54.98^{c} | 69.67^{a} | 34.10^{d} |
| C14 | 1 | 31.87^{a} | 99.96^{d} | 76.03^{a} | 43.80^{c} |
| | 0.25 | 44.40^{a} | 94.51^{d} | 72.05^{a} | 49.70^{b} |
| | 0.0625 | 39.47^{a} | 81.38^{c} | 67.56^{b} | 45.90^{b} |
| C60 | 1 | 24.15^{b} | 65.45^{d} | 62.79^{a} | 36.30^{a} |
| | 0.25 | 41.86^{a} | 64.78^{d} | 51.46^{b} | 44.50^{a} |
| | 0.0625 | 43.84^{a} | 86.56^{b} | 72.96^{a} | 33.40^{b} |
| C66 | 1 | 28.12^{d} | 69.22^{d} | 51.14^{f} | 34.90^{b} |
| | 0.25 | 31.78^{b} | 51.21^{f} | 71.10^{a} | 17.00 |
| | 0.0625 | 35.05^{a} | 70.64^{f} | 44.37^{d} | 15.20 |

^{a}Inhibitory effect is statistically significant (P < 0.001)
^{b}Inhibitory effect is statistically significant (P < 0.005)
^{c}Inhibitory effect is statistically significant (P < 0.01)
^{d}Inhibitory effect is statistically significant (P < 0.02)
^{e}Inhibitory effect is statistically significant (P < 0.025)
^{f}Inhibitory effect is statistically significant (P < 0.05)

TABLE 3J
Effect of S-oligos on proliferation of human colon cancer cells

| | | DLD-1 | HT-29 | LoVo |
|---|---|---|---|---|
| Oligo | Dose (μM) | % Inhibition | | |
| C5 | 1 | 61.28^{a} | 51.04^{a} | 97.52^{a} |
| | 0.25 | 62.60^{a} | 47.12^{a} | 101.60^{a} |
| | 0.0625 | 41.61^{a} | 52.68^{a} | 75.62^{a} |
| C9 | 1 | 45.57^{a} | 59.48^{a} | 84.93^{a} |
| | 0.25 | 48.51^{a} | 58.95^{a} | 66.75^{a} |
| | 0.0625 | 48.06^{a} | 27.82^{e} | 35.90^{e} |
| C11 | 1 | 55.23^{a} | 43.39^{a} | 59.40^{b} |
| | 0.25 | 65.40^{a} | 29.25^{b} | 96.19^{a} |
| | 0.0625 | 52.22^{a} | 42.61^{a} | 68.53^{a} |
| C14 | 1 | 48.13^{a} | 64.15^{a} | 104.88^{a} |
| | 0.25 | 54.37^{a} | 47.08^{a} | 75.35^{a} |
| | 0.0625 | 41.87^{a} | 32.12^{e} | 37.23^{c} |
| C60 | 1 | 46.19^{b} | 24.92^{b} | 21.54 |
| | 0.25 | 49.14^{a} | 18.98^{d} | 16.67 |
| | 0.0625 | 35.97^{a} | 20.01^{f} | 29.25 |
| C66 | 1 | 45.78^{d} | −31.32 | 5.58 |
| | 0.25 | 37.29^{b} | −35.32 | −1.77 |
| | 0.0625 | 19.98^{a} | −8.92 | 8.42 |

^{a}Inhibitory effect is statistically significant (P < 0.001)
^{b}Inhibitory effect is statistically significant (P < 0.005)
^{c}Inhibitory effect is statistically significant (P < 0.01)
^{d}Inhibitory effect is statistically significant (P < 0.02)
^{e}Inhibitory effect is statistically significant (P < 0.025)
^{f}Inhibitory effect is statistically significant (P < 0.05)

TABLE 3K
Effect of S-oligos on proliferation of human lung cancer cells

| Cell Line | | Calu-1 | A549 | A427 | NCI-H460 |
|---|---|---|---|---|---|
| Oligo | Dose (μM) | % Inhibition | | | |
| C5 | 1 | 55.11^{c} | 95.48^{a} | 34.49^{b} | 33.05^{b} |
| | 0.25 | 35.04^{b} | 93.59^{a} | 42.15^{a} | 64.56^{a} |
| | 0.0625 | 26.46^{b} | 84.51^{a} | 19.70^{d} | 66.22^{a} |
| C9 | 1 | 42.17^{b} | 81.76^{a} | 51.26^{a} | 58.72^{a} |
| | 0.25 | 33.29^{a} | 80.02^{a} | 38.77^{b} | 64.79^{a} |
| | 0.0625 | 34.50^{a} | 75.69^{a} | 23.48^{d} | 59.32^{a} |
| C11 | 1 | 52.07^{a} | 96.96^{a} | 29.17^{b} | 53.33^{a} |
| | 0.25 | 47.57^{a} | 93.75^{a} | 39.03^{a} | 55.04^{a} |
| | 0.0625 | 48.51^{a} | 82.68^{a} | 37.95^{e} | 66.11^{a} |
| C14 | 1 | 65.11^{a} | 90.67^{a} | 44.68^{a} | 72.82^{a} |
| | 0.25 | 57.94^{a} | 83.16^{a} | 52.51^{a} | 76.45^{a} |
| | 0.0625 | 49.45^{a} | 72.29^{a} | 26.73^{b} | 60.64^{a} |
| C60 | 1 | 52.46^{a} | 76.62^{a} | 34.49^{f} | 67.50^{a} |
| | 0.25 | 46.23^{a} | 76.27^{a} | 31.9^{c} | 66.90^{a} |
| | 0.0625 | 45.01^{a} | 66.03^{a} | 36.24^{f} | 60.09^{a} |
| C66 | 1 | 57.26^{a} | 80.22^{a} | 45.50^{b} | 66.21^{a} |
| | 0.25 | 52.43^{a} | 61.70^{a} | 45.34^{a} | 64.20^{a} |
| | 0.0625 | 53.85^{a} | 60.01^{a} | 25.55^{f} | 60.27^{a} |

^{a}Inhibitory effect is statistically significant (P < 0.001)
^{b}Inhibitory effect is statistically significant (P < 0.005)
^{c}Inhibitory effect is statistically significant (P < 0.01)
^{d}Inhibitory effect is statistically significant (P < 0.02)
^{e}Inhibitory effect is statistically significant (P < 0.025)
^{f}Inhibitory effect is statistically significant (P < 0.05)

TABLE 3L
Effect of S-oligos on proliferation of human ovarian cancer cells

| | | |
|---|---|---|
| Cell Line | OVCAR-3 | SKOV-3 |

| | | |
|---|---|---|
| Oligo | Dose (μM) | % Inhibition |

| | | | |
|---|---|---|---|
| C5 | 1 | 62.39^{b} | 7.64 |
| | 0.25 | 51.08^{c} | 13.92 |
| | 0.0625 | 31.96^{d} | 16.50 |
| C9 | 1 | 26.63 | 15.20 |
| | 0.25 | 28.09 | 15.16 |
| | 0.0625 | 17.01 | 16.80 |
| C11 | 1 | 16.78 | 14.35 |
| | 0.25 | 27.73^{f} | 16.86 |
| | 0.0625 | 18.24 | 17.86 |
| C14 | 1 | 32.83^{f} | 17.23 |
| | 0.25 | 34.29^{f} | 16.62 |
| | 0.0625 | 16.02 | 18.02 |
| C60 | 1 | −26.27 | 16.48 |
| | 0.25 | 6.20 | 16.75 |
| | 0.0625 | −4.52 | 16.50 |
| C66 | 1 | 20.28 | 20.20^{a} |
| | 0.25 | −1.10 | 15.65 |
| | 0.0625 | 8.03 | 25.57^{b} |

^{a}Inhibitory effect is statistically significant (P < 0.001)
^{b}Inhibitory effect is statistically significant (P < 0.005)
^{c}Inhibitory effect is statistically significant (P < 0.01)
^{d}Inhibitory effect is statistically significant (P < 0.02)
^{f}Inhibitory effect is statistically significant (P < 0.05)

Twenty candidate Apoptotic Sequences in Table 3A were selected to prepare targeting agent oligonucleotides to conduct cell death tests similar to those described above. The selected agents targeting Apoptotic Sequences were introduced into phosphorothioated DNA and prepared in commercially available lipids for transfection, the lipids being a standard transfection technique for in vitro antisense DNA tests. The resulting Apoptotic Sequence targeting agent compositions were applied to cell cultures grown from a tumor removed from Subject R. Table 4 shows the results, including healthy and cancer cell death percentages. Blanks indicate results in which substantial amounts of both healthy cells and cancer cells were killed.

TABLE 4
Candidate Apoptotic Sequence Cell Death Tests

| | | | | |
|---|---|---|---|---|
| | | | | Subject |
| SEQ | | | | R colon |
| ID | | Phosphorothioated | Healthy | cancer |
| NO: | ID | Apoptotic Sequence | cells | cells |

| | | | | |
|---|---|---|---|---|
| 1 | 5 | AAGGGGGTTCCTTGGGC | 10% | 82% |
| 6 | 8 | GCTCAGGTTTGCTCAGG | 28% | 46% |
| 2 | 9 | GGCCTGCCAGAAGCACA | 9% | 70% |
| 8 | 10 | CCAACTGGATCCCAGGT | | |
| 7 | 11 | TGGTGGCCACGCATGCG | 20% | 75% |
| 9 | 12 | CGGATGTCCCTGCTGGG | | |
| 3 | 14 | GCCGATTAACACCAGCC | 15% | 72% |
| 10 | 16 | GCCGATTCACACCCAGC | | |
| 11 | 20 | GCCTCGTACCTAGCCG | | |
| 12 | 23 | CGCCTCGGCCGATTAAC | | |
| 13 | 26 | GGCCGATTTACACCCGG | | |
| 14 | 30 | TCGGCCGATTAACCCCA | | |
| 15 | 31 | CCGATTAACACCGGCCT | | |
| 16 | 33 | GCTGTTGTCATACTTGCT | | |
| 17 | 35 | CCACGTGATGTAGACTG | | |
| 18 | 53 | CCCAGCCTCGTACCTAG | | |
| 19 | 55 | CAGCCTCTACCTAGCCTT | | |
| 20 | 57 | CACCGGCCTCGTACCT | | |
| 4 | 60 | CGATTAACCACCGGCCT | 12% | 73% |
| 5 | 66 | TTGAACCCTAGGCATGT | 8% | 83% |

Although all of the sequences causing cancer cell death in Tables 3G-L also showed evidence of causing some healthy cell death, it is difficult to determine low cell death percentages such as those shown in the tables.

### Example 6

### In Vivo Mouse Toxicity Testing

Because healthy cell death is a direct reflection of toxicity, fifteen female C57BL/6NTaC strain, 12-14 week old mice were administered S-oligos 5, 9, 11, 60 and 66 or a PBS control at a concentration of 1 mg/ml. Each S-oligo was injected at 250 ug/ml into three mice per set for testing toxic effects of each Apoptotic Sequence targeting agent (equivalent to 5 mg per kg body weight). There was no apparent change in mouse behavior or other traits over two weeks of observation.

A subsequent test on five mice with an increased dose of each S-oligo using an equivalent of 50 mg S-oligo per kg of body weight was performed (2 mg of oligo/ml). As in the prior test, after several weeks no apparent change in mouse behavior was observed.

Thus it appears safe to administer doses of Apoptic Sequence targeting agent composition between approximately 5 mg-50 mg DNA per kg of body weight. Dosage may also be limited to no more than approximately 25 mg DNA per kg body weight. Because mice are standard toxicity model for humans, these dosages may be appropriate for administration to a human as well.

### Example 7

### Apoptotic Sequence Compositions

FIG. 7 shows four Apoptotic Sequence targeting agent compositions corresponding to Apoptotic Sequence numbers 5, 9, 60 and 66. Each of these compositions is administered to a cancer cell, including a cancer cell in a human subject, singly or in conjunction with one or more additional compositions. When applied to a human subject, they are used alone or combined with other therapeutics, such as chemotherapeutics and radiotherapeutics, or other treatments, such as surgery to remove a tumor, or injection into a tumor or its blood supply, or in proximity to a tumor.

A typical low dose of an Apoptotic Sequence based treatment formulation for an average human includes about 300 mg of phosphorothioated DNA, and a high dose includes about 1500 mg. The treatment formulation is administered weekly. It includes one or a combination of multiple Apoptotic Sequence targeting agent formulations. Administration continues until no further signs of cancer are detected and is resumed in cancer signs reappear if necessary. Tumor markers, such as those corresponding to the Apoptic Sequences are measured after each administration and administered treatment formulations is adjusted based on observed results.

An example of a complete administration formula and protocol for administration of one or more Apoptotic Sequence targeting agent formulations to one human subject includes the following steps. First, approximately 300 mg cGMP phosphorothioated DNA having an Apoptic Sequence is ordered from any commercial source or prepared. It is used either in desalted or HPLC purified form. The phosphorothioated DNA is quite stable when stored at −20° C. in the lyophilized form. It is stable for one week when stored at 4° C. Second, sterile PBS (phosphate buffered saline) or artificial CSF (cerebrospinal fluid) is provided. Third, the 300 mg of phosphorothioated DNA is prepared with 30 ml of sterile PBS or artificial CSF to form an Apoptotic Sequence based treatment formulation. These are mixed by shaking gently on a nutator at 4° C. or gently pipetting up and down at 4° C. Finally, the Apoptotic Sequence based treatment formulation is administered to a subject by slow IV drip for 30 minutes alone or in combination with other formulations (FIG. 7).

Each Apoptic Sequence treatment formulation should be undetectable in the body after 48 hours. Effects on cancer cells may be detectable within 24 hours of administration.

The Apoptotic Sequence based formulations have little to no effects on healthy tissues, such as liver toxicity.

### Example 8

### Efficacy of a Candidate Apoptotic Sequence In Vitro and In Vivo

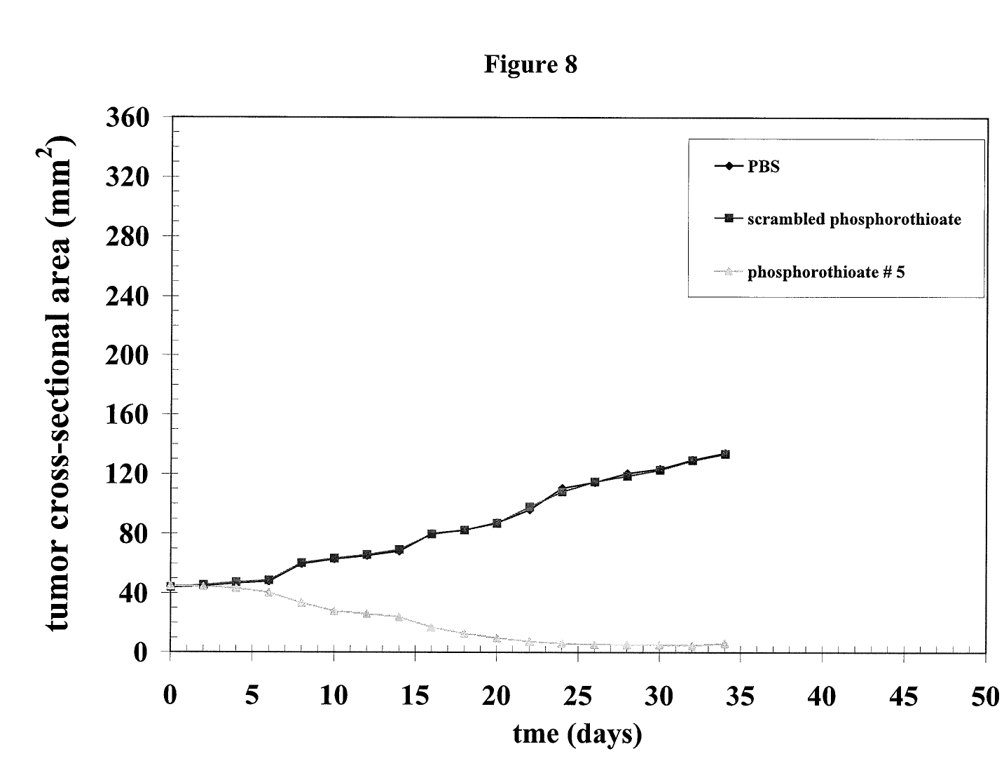

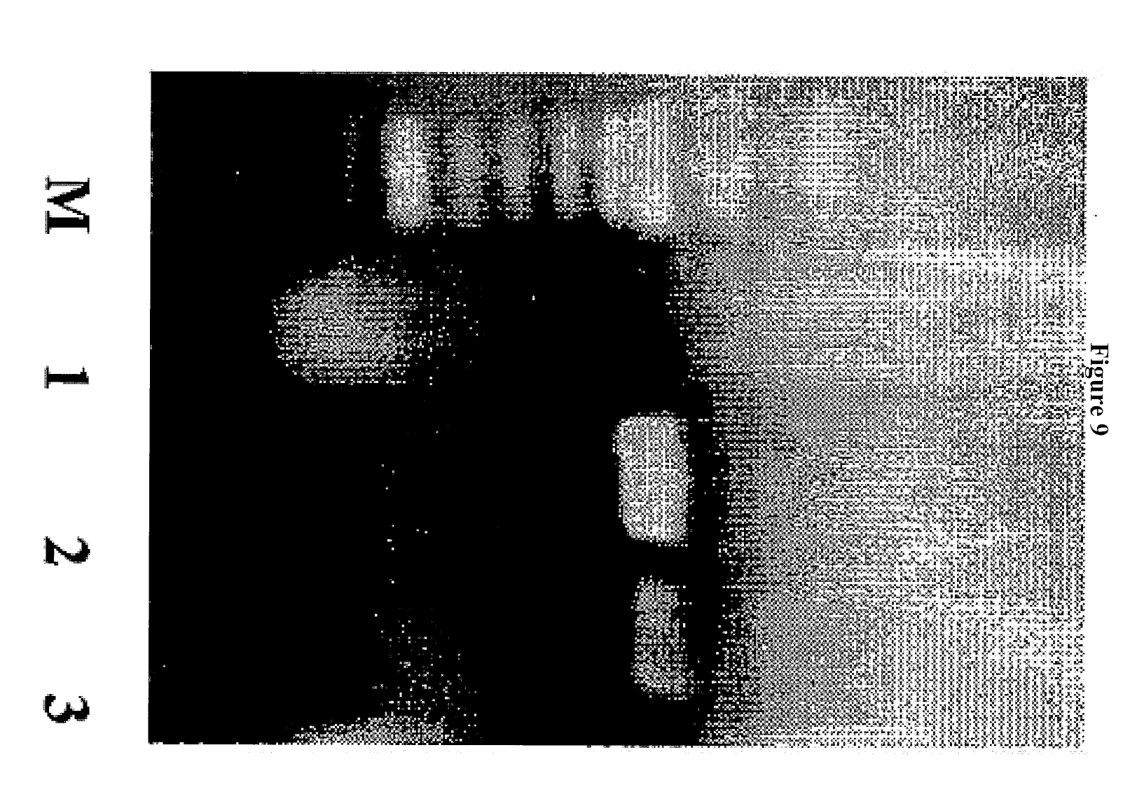

Studies indicate cancer specific expression of the SET-1 gene (SET domain protein-1; Terranova et al., Gene 2002; 292(1-2):33-41; Apoptotic Sequence 5; Tables 2 and 5), cancer-specific apoptosis by SET-1 gene depletion (Apoptotic Sequence 5; Tables 3G, 4, 5), and activity of SET-1 antisense in colon cancer xenografts (FIG. 8). The SET domain is a 130-amino acid, evolutionarily conserved sequence motif present in chromosomal proteins that function in modulating gene activities from yeast to mammals. Initially identified as members of the Polycomb- and trithorax-group (Pc-G and trx-G) gene families, which are required to maintain expression boundaries of homeotic selector (HOM-C) genes, SET domain proteins are also involved in position-effect-variegation (PEV), telomeric and centromeric gene silencing, and possibly in determining chromosome architecture (Reviewed in Jenuwein et al., Cell Mol Life Sci. 1998; 54:80-93; Peters et al., Cell 2001; 107:323-327; Schneider et al., Trends Biochem. Sci. 2002; 27:396-402; Varambally et al., Nature 2002; 419:624-629; Caldas and Aparicio, Cancer Metastasis Rev 1999; 18:313-329). SET domain proteins methylate histones irreversibly and influence the transcriptional state of genes and are essential for the epigenetic maintenance of either repressed or activated transcriptional states (Marmorstein, Trends Biochem. Sci. 2003; 28:59-62; Turner, Cell 2002; 111:285-291; Peters et al., Cell 2001; 107:323-327). There have been several SET domain genes that have been implicated in cancer (Marmorstein, 2003, supra; Turner, Cell 2002; 111:285-291; Peters et al., Cell 2001; 107:323-327; Schneider et al., Trends Biochem. Sci. 2002; 27:396-402; Varambally et al., Nature 2002; 419:624-629; Caldas and Aparicio, Cancer Metastasis Rev 1999; 18:313-329). These observations implicate SET domain proteins as multifunctional chromatin regulators with activities in both eu- and hetero-chromatin a role consistent with their modular structure, which combines the SET domain with additional sequence motifs of either a cysteine-rich region/zinc-finger type or the chromo-domain. SET domain biology is an important area of research since proteins bearing SET domains are widely distributed and play putative roles in epigenetic regulation and carcinogenesis. Most recently, SET domain, EZH2 polycomb group protein HMT has been shown to be overexpressed in metastatic prostate cancer (Varambally et al., 2002, supra) and the SET domain of MLL has been implicated in leukemogenesis (Caldas and Aparicio, 1999, supra).

The putative role of SET domain proteins in epigenetic regulation and the involvement of these proteins in various cancers indicates that specific down-regulation of SET-1 (Terranova et al., Gene 2002; 292(1-2):33-41) could be developed as a specific anticancer therapy. SET-1 depleting antisense phosphorothioate deoxynucleotide (Apoptotic Sequence 5, SEQ ID NO:1) was tested for specificity, non-toxicity, and effectiveness as a anticancer therapy. Identification of the SET-1 sequence unique to cancer cells was realized by designing oligonucleotides from conserved sequences of the flanking regions of the SET-1 mRNA as described above. RT-PCR-competition assays were performed using total RNA from normal and tumor tissues and cancer patient blood, and levels of the specific SET-1 oligonucleotide (SEQ ID NO:1) in the RNA of normal and tumor tissues and blood were determined. Total RNA from control, tumor and blood of a colon cancer patient was reverse transcribed and PCR amplified for 35 cycles. The intensity of the bands visible after agarose gel electrophoresis of the PCR reaction products (FIG. 9) was quantified. These results suggested cancer-specific expression of SET-1. The amplification product was seen only in amplification templates from colon cancer patient tumor sample (FIG. 9, lane 2) or blood (FIG. 9, lane 3) and not in a control sample (FIG. 9, lane 1). The intensity of PCR product band is 3-fold higher in the tumor as compared with the blood of the cancer patient, and is not present in the normal sample.

In vitro testing of phosphorothioate oligonucleotides was performed on normal (human aorta vascular smooth muscle cells) and the colon cancer (from the tumor of a colon cancer patient) cultured cells to identify promising genetic targets characteristic for each tumor histology. Phosphorothioate DNA against the sequence of SET-1 (SEQ ID NO:1) that was found to appear unique in cancer cells and not in the normal cells was transfected into the cultured cancer and normal cell lines using standard methods described above. The IC₅₀ value was determined using an MTT assay. In parallel, total RNA was isolated from cultured cells after transfection with the phosphorothioate DNA and the RT-PCR competition assay were performed using the corresponding oligonucleotide. The results obtained show that phosphorothioate DNA against SET-1 killed 80-85% of the cancer cells whereas only 10% normal cells were killed using the same amount of these phosphorothioate DNA molecules (Table 5). These studies indicated that SET-1 depletion was specifically toxic to cancer cells. Each value is the average of 8 independent determinations obtained by transfecting the cultured cells with 5 pg/ml phosphorothioate DNA, the concentration found to show optimal effect. Optimum concentration was determined for each cell lines by transfecting the cultured cells with varying concentrations of phosphorothioate DNA ranging between 1-10 pg/ml.

Lack of animal toxicity of the agent targeting Apoptotic Sequence 5 (SEQ ID NO:1) was demonstrated by injecting the oligonucleotide intraperitoneally (i.p.) into C57B mice at 7 mg/kg or 70 mg/kg as a single i.p. dose. The animals survived and had normal 15% weight gain over the next 1 month. These studies indicated lack of animal toxicity. Thus systemic delivery of an Apoptotic Sequence targeting agent has no apparent toxic effects in vivo.

Activity of an agent targeting Apoptotic Sequence 5 in SW480 colon cancer cell xenografts was performed by implanting 2×10⁶ cells subcutaneously in nu/nu nude mice. Once the tumors had reached a surface area of 45 mm², animals were treated with a single i.p. injection of PBS, the targeting agent to apoptotic Sequence 5 (SEQ ID NO:1), or scrambled control. Daily bidimensional measurements were carried out and cross-sectional tumor area data is presented as a function of time in FIG. 8. Results of these studies clearly demonstrate that Apoptotic Sequence 5 targeting agent is very active in this human cell line xenograft model. These findings present a persuasive case for development of therapeutics based on Apoptotic Sequence 5 as an anticancer agent. Thus systemic delivery of an Apoptotic Sequence based agent in addition to having no apparent toxic effect in vivo is effective in treating a cancer.

### Example 9

### Efficacy of Targeting Agents to Candidate Apoptotic Sequences Used Singly or in Combination In Vivo

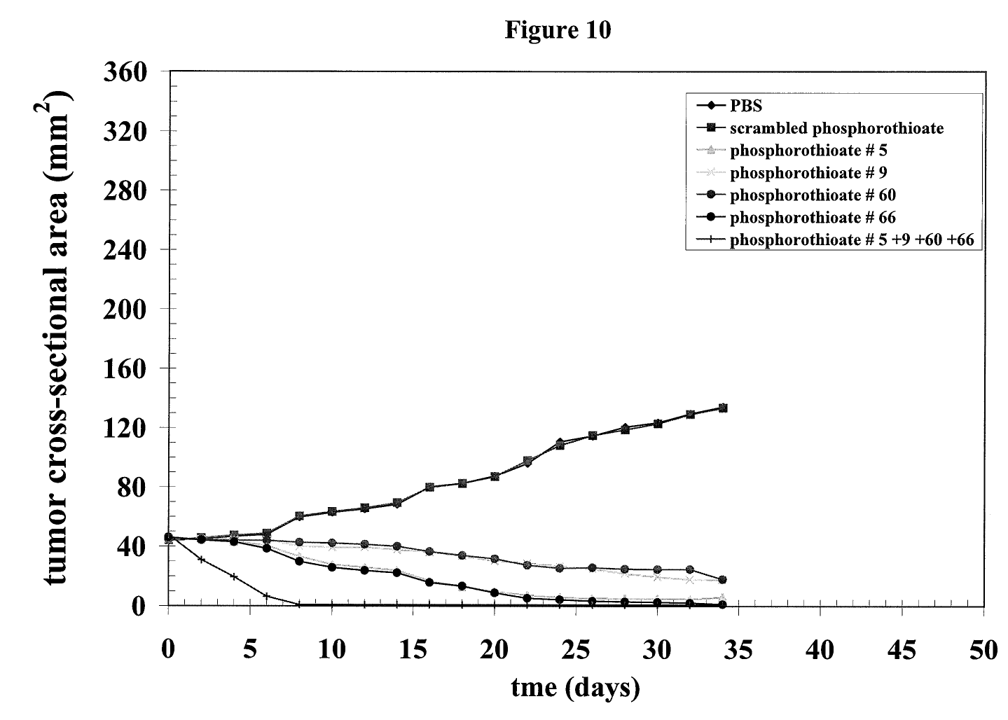

Activity of Apoptotic Sequence 5, 9, 60, 66, and a mixture of all four-based targeting agents were tested in SW480 colon cancer cell xenografts. The experiment was performed by implanting 2×10⁶ cells in 100 μl of PBS subcutaneously in 20 week old athymic nu/nu nude mice. Tumors were allowed to develop (˜25 days) prior to initiation of therapy. Once the tumors had reached a surface area of ˜22 mm², animals were treated with a single i.p. injection of PBS, s-oligonucleotides based on Apoptotic Sequences 5, 9, 60, 66 and a mixture of all four, scrambled control and PBS control. Bidimensional measurements were carried out and cross-sectional tumor area data determined which is presented as a function of time in FIG. 10. Results of these studies demonstrate that targeting all four Apoptotic Sequences results in potent therapeutic anti-cancer activity in this colon carcinoma human cell line xenograft model. These findings present a persuasive case for development of all four Apoptotic Sequences as anticancer therapeutic targets, particularly for treating colon cancer. Of particular note was a higher efficacy and more rapid activity of treatments which combined the four Apoptotic Sequences targeting agents compared to use of each alone. Combined treatment compositions and Apoptotic Sequence 5 and 66 based agents used alone, resulted in almost complete tumor regression at the end of the experimental cycle (35-days).

### Example 10

### Presence of a Single Target Apoptotic Sequence in Multiple Genes

Several Cancer Marker Sequences were identified, a subset of which, when targeted were cancer cell death inducing Apoptotic Sequences. Of particular note was the presence of Apoptotic Sequence targets in multiple tissues, cancers and diverse genes as seen in the examples listed in Table 5.

TABLE 5

Candidate Apoptotic Sequence Computational
Analysis

| | Candidate | |
|---|---|---|
| ID | Apoptotic Sequence | Affected Cancers |
| 5 | + GCCCAAGGAACCCCCTT | ovarian colorectal |
| | (SEQ ID NO: 21) | brain epid testis |
| | − AAGGGGGTTCCTTGGGC | liver |
| | (SEQ ID NO: 1) | |

Targeted Genes
CHCHD3(7) EEF1G(11) LOC136337(X) ABCC3(17)

| | | |
|---|---|---|
| 8 | + GCTCAGGTTTGCTCAGG | ovarian colorectal |
| | (SEQ ID NO: 22) | lung testis liver |
| | − CCTGAGCAAACCTGAGC | skin |
| | (SEQ ID NO: 6) | |

Targeted Genes
LTBR(12)

| | | |
|---|---|---|
| 9 | + TGTGCTTCTGGCAGGCC | breast colorectal |
| | (SEQ ID NO: 23) | brain adrenal eye |
| | − GGCCTGCCAGAAGCACA | |
| | (SEQ ID NO: 2) | |

Targeted Genes
GNB2L1(5)

| | | |
|---|---|---|
| 10 | + ACCTGGGATCCAGTTGGAGGACGG | colorectal lung brain |
| | C | |
| | (SEQ ID NO: 24) | |
| | − GCCGTCCTCCAACTGGATCCCAGG | |
| | T | |
| | (SEQ ID NO: 25) | |

Targeted Genes
ZNF500(16)

| | | |
|---|---|---|
| 11 | + CGCATGCGTGGCCACCA | colorectal brain |
| | (SEQ ID NO: 7) | lymph |
| | − TGGTGGCCACGCATGCG | |
| | (SEQ ID NO: 26) | |

Targeted Genes
LOC388707(1) LAMR1(3) LOC389672(8)

| | | |
|---|---|---|
| 12 | + CCCAGCAGGGACATCCG | ovarian colorectal |
| | (SEQ ID NO: 27) | lung cervix uterus |
| | − CGGATGTCCCTGCTGGG | skin pancreas testis |
| | (SEQ ID NO: 9) | liver |

Targeted Genes
MOV10(1)

| | | |
|---|---|---|
| 13 | + GGCTAGGTACGAGGCTGG | ovarian colorectal |
| | (SEQ ID NO: 28) | lung brain uterus |
| | − CCAGCCTCGTACCTAGCC | skin kidney pancreas |
| | (SEQ ID NO: 29) | muscle lymph eye |

Targeted Genes
AACS(12) AAMP(2) ABCF3(3) ACTB(7) ACTBP2(5)
ACTG1(17) ACTN1(14) ADCK4(19) ADPRT(1) AES(19)
AFG3L2(18) AHSA1(14) AIPL1(17) AKT1(14) ALDOA(16)
ANAPC2(9) ANKRD19(9) ANXA11(10) ANXA7(10)
AP1M1(19) AP2A1(19) AP2M1(3) APCL(19) APOE(19)
ARHGDIA(17) ARHGEF1(19) ARHGEF16(1) ARL6IP4(12)
ARPC2(2) ASPH(8) 11ASRGL1(11) ASS(9) ATF4(22)
ATF5(19) ATP1A1(1) ATP5A1(18) ATP5F1(1) ATP5O(21)
AUTL2(X) AZ2(3) bA395L14.12(2) BAT3(6) BCAS3(17)
BLP1(8) BRMS1(11) BSG(19) BTF3(5) C10orf45(10)
C14orf126(14) C20orf41(20) 2orf17(2) C3orf4(3)
C4orf9(4) C5orf6(5) C6.1A(X) C6orf107(6) 6orf11(6)
C6orf48(6) C7orf30(7) CACNA2D3(3) CAMKK2(12)
CASP4(11) CASQ1(1) CBS(21) CBX7(22) CBX8(17)
CCND3(6) CCT3(1) CCT5(5) CCT6A(7) CCT7(2) CD74(5)
CD79A(19) CD79B(17) CDC20(1) CDC2L2(1) CDCA5(11)
CDCA8(1) CDH12(5) CDH24(14) CDIPT(16) CDK4(12)
CDW92(9) CEECAM1(9) CENPB(20) CGI-96(22) CHCHD3(7)
CIDEB(14) CNOT10(3) COMT(22) ORO1A(16) CORO2A(9)
COTL1(16) CRN(4) CRTAP(3) CRYBB2P1(22) CS(12)
CTAG3(6) CYB5-M(16) DBH(9) DBI(2) DCLRE1C(10)
DCTN2(12) DDB1(11) DDX10(11) DDX56(7) DGCR8(22)
DGKA(12) DHCR24(1) DKFZp434B227(3) DKFZP434C171(5)
DKFZP434K046(16) DKFZP564D172(5) DKFZp5G4K142(X)
DKFZp586M1819(8) DNAJB1(19) DNCH1(14) DNM2(19)
DRIM(12) DustyPK(1) E1B-AP5(19) E2F4(16)
EDARADD(1) EEF1D(8) EEF1G(11) EEF2(19) EIF2B5(3)
EIF2S1(14) eIF3k(19) EIF3S1(15) EIF3S2(1)
EIF3S5(11) EIF3S7(22) EIF3S8(16) EIF3S9(7)
EIF4G1(3) ELMO2(20) ENDOG(9) ENO1(1) ENO1P(1)
ENTPD8(17) EPAC(12) ETFDH(4) FAH(15) FAM31B(1)
FANCA(16) FBL(19) FBXO7(22) FDFT1(8) FECH(18)
FGFR4(5) FKBP1B(2) FKBP8(19) FKSG17(8) FLI1(11)
FLJ00038(9) FLJ10241(19) FLJ12750(12) FLJ12875(1)
FLJ14800(12) FLJ14827(12) FLJ20071(18) FLJ20203(1)
FLJ20294(11) FLJ20487(11) FLJ21827(11)
FLJ22028(12) FLJ22688(19) FLJ25222(15)
FLJ27099(14) FLJ31121(5) FLJ32452(12) FLJ35827(11)
FLJ38464(9) FLJ44216(5) FMN2(1) FMO5(1) FOSL1(11)
FSCN1(7) FUS(16) G22P1(22) G2AN(11) GA17(11)
GALK2(15) GAPD(12) GCC1(7) GCDH(19) GDI2(10)
GA1(22) GGCX(2) GIT1(17) GLUL(1) GNB2L1(5)
GOLGB1(3) GPAA1(8) GPI(19) GRHPR(9) GRSF1(4)
GSPT1(16) GSTM4(1) GYS1(19) H3F3B(17) HAND1(5)
HARS2(20) HAX1(1) HCA127(X) HCCR1(12) HCG4(6)
HDAC1(1) HDLBP(2) HLA-B(6) HMGA1(6) HMGA1L3(12)
HMGN1(21) HMGN2(1) HNRPD(4) HNRPH3(10) HNRPU(1)
HPS4(22) HRMT1L1(21) HS3ST4(16) HSA9761(5)
HSPA9B(5) HSPB1(7) HSPC142(19) HSPC242(22)
HSPCB(6) HSPCP1(4) HSPD1(2) ID3(1) IER3(6)
IGFBP4(17) IGHV4-34(14) L1RL1LG(19) ILF2(1)
ILVBL(19) IMPDH2(3) ITGB4BP(20) JIK(12) JM4(X)
K-ALPHA-1(12) KCNN2(5) KCTD1(18) KHSRP(19)
KIAA0141(5) KIAA0182(16) KIAA0258(9) KIAA0582(2)
KIAA0774(13) KIAA1049(16) KIAA1055(15)
KIAA1115(19) KIAA1211(4) KIAA1765(3) KNS2(14)
KPNB1(17) KRT17(17) KRT5(12) KRT8(12) LAMR1P3(14)
LARGE(22) LASP1(17) LCP1(13) LDHB(12) LDHBP(X)
LENG5(19) LGALS1(22) LGALS3BP(17) LIMK2(22)
LIN28(1) LMO7(13) LOC113174(11) LOC127253(1)
LOC129138(22) LOC136337(X) LOC137829(1)
LOC144581(12) LOC145414(14) LOC145989(15)
LOC146253(16) LOC148640(1) LOC149501(1)
LOC150417(22) LOC158078(9) LOC192133(14)
LOC201292(17) LOC220717(2) LOC221838(7)
LOC253482(9) LOC266724(2) LOC266783(1)
LOC283747(15) LOC283820(16) LOC284089(17)
LOC284393(19) LOC285214(3) LOC285741(6)
LOC285752(6) LOC286444(X) LOC339395(1)
LOC339799(2) LOC342705(18) LOC348180(16)
LOC374443(12) LOC387703(10) LOC388076(15)
LOC388344(17) LOC388519(19) LOC388556(19)
LOC388642(1) LOC388654(1) LOC388968(2)
LOC389181(3) LOC389240(4) LOC389342(5)
LOC389849(X) LOC389901(X) LOC390415(13)
LOC390814(17) LOC390860(18) LOC391634(4)
LOC391717(4) LOC391739(5) LOC391800(5)
LOC399942(11) LOC399969(11) LOC400068(12)
LOC400586(17) LOC400634(17) LOC400744(1)
LOC400954(2) LOC400963(2) LOC401010(2)
LOC401146(4) LOC401245(6) LOC401316(7)
LOC401677(11) LOC401838(16) LOC402057(22)
LOC402142(3) LOC402259(7) LOC402579(7)
LOC402650(7) LOC51149(5) LOC91272(5) LOC92755(8)
LPPR2(19) LSP1(11) LU(19) LYGE(8) M6PRBP1(19)
MAGED1(X) MAMDC2(9) MAP3K4(6) MAPRE1(20) MARS(12)
MBD3(19) MCM2(3) MECP2(X) MESDC1(15) MFGE8(15)
MGAT4B(5) MGC10540(17) MGC10986(17) MGC11061(2)
MGC12966(7) MGC19764(17) MGC20446(11) MGC26O1(16)
MGC2714(11) MGC2749(19) MGC29816(8) MGC3162(12)
MGC35555(8) MGC4606(16) MGC48332(5) MGC52000(2)
MGC5508(11) MGC71999(17) MGST2(4) MRPL2(6)
MRPL28(16) MRPL9(1) MRPS12(19) MRPS27(5)
MRPS34(16) MSH3(5) MSH6(2) MSN(X) MSNL1(5)
MUS81(11) MVP(16) MYBL2(20) MYCT1(6) NACA(12)
NAP1L1(12) NARF(17) NARS(18) NCOA4(10) NDE1(16)
NDUFA10(2) NDUFAB1(16) NDUFB9(8) NDUFS1(2)
NDUFS2(1) NICE-3(1) NICE-4(1) NME1(17) NME3(16)
NONO(X) NPM1(5) NQO2(6) NRBF2(10) NRBP(2) NS(3)
NUDT8(11) NUP210(3) NUTF2(16) NUTF2P2(14) NXF1(11)
OAZ1(19) OK/SW-cl.56(6) OS-9(12) OSBPL9(1) PBP(12)
PCCA(13) PCOLCE2(3) PDAP1(7) PDHA1(X) PDXP(22)
PEA15(1) PECI(6) Pfs2(16) PGD(1) PGK1(X) PH-4(3)
PHGDH(1) PIGT(20) PIK4CA(22) PKD1P3(16) PKM2(15)
PKM2(15) PLEKHA4(19) PM5(16) PMM2(16) POLDIP3(22)
POLE3(9) POLH(6) POLR2E(19) POLR2R(3) POU2F1(1)
PPFIBP2(11) PPIE(1) PPOX(1) PPP1R15A(19) PPP1R8(1)
PPP2R1A(19) PPP4C(16) PRAME(22) PRDX1(1)
PRKACA(19) PRNPIP(1) PRO1855(17) PRPF31(19)
PSAP(10) PSMC2(7) PSMD2(3) PSME1(14) PSPC1(13)
PTBP1(19) PTPN6(12) PTPRCAP(11) PTPRD(9) PTPRG(3)
PTTG1IP(21) PYCR1(17) RAB32(6) RAE1(20) RALGDS(9)
RAN(12) RANP1(6) RARS(5) RASAL1(12) RBBP7(X)
RDH11(14) REC14(15) RER1(1) RFC2(7) RGS16(1)
RHEBL1(12) RIOK1(6) RNF10(12) RNF20(9) RNF8(6)
RoXaN(22) RPL10(X) RPL10P1(21) RPL13(16) RPL14(3)
RPL15(3) RPL15P2(14) RPL24(3) RPL28(19) RPL3(22)
RPL30(8) RPL35(9) RPL35A(3) RPL37A(2) RPL37AP1(20)
RPL5(1) RPL8(8) RPL9(4) RPLP0(12) RPLP0P2(11)
RPLP2(11) RPS10(6) RPS14(5) RPS15(19) RPS16(19)
RPS17(15) RPS17P2(5) RPS19(19) RPS19P1(20)
RPS2(16) RPS20(8) RPS20P3(5) RPS2L1(20) RPS3(11)
RPS6(9) RPS9(19) RPS9P2(22) RRP4(9) RRP40(9)
RTKN(2) RUVBL1(3) RUVBL2(19) S100A16(1) SAFB(19)
SARS(1) SART3(12) SATB1(3) SBDS(7) SCD(10)
SCYL1(11) SEC31L1(4) SFRS2(17) SH2D3A(19)
SH3BP1(22) SH3BP5(3) SHMT2(12) SIAHBP1(8)
SIN3A(15) SKB1(14) SLC25A3(12) SLC25A6(X)
SLC25A6(Y) SLC7A5(16) SMARCA4(19) SMARCB1(22)
SNRPA(19) SNRPA1(15) SNRPB(20) SNRPC(6) SNX17(2)
SNX6(14) SOD1(21) SPINT1(15) SPPL2B(19) SRP14(15)
ST7(7) STAG3(7) STAMBP(2) STARD7(2) STAT6(12)
STIM1(11) STK33(11) STMN1(1) STXBP2(19)
SUPT16H(14) SUPT5H(19) SV2A(1) SV2C(5) TADA2L(17)
TADA3L(3) TAF11(6) TAGLN2(1) TCEB1(8) TCL1A(14)
TD-60(1) TDPX2(9) TIC(2) Tino(19) TIP120A(12)
TK1(17) TMEM4(12) TMSB4X(X) TOR3A(1) TPI1(12)
TPK1(7) TPM3(1) TRAP1(16) TRAPPC1(17) TRAPPC3(1)
TRBC2(7) TRIP10(19) TRP14(17) TUBA3(12) TUBA6(12)
TUBB2(9) TUSC2(3) TXNDC5(6) TXNIP(1) UBAP2(9)
UBC(12) UBE2J2(1) USP11(X) USP7(l6) VAMP8(2)
VWF(12) VWFP(22) WAC(10) WBSCR1(7) WDR1(4)
WDR18(19) WDR34(9) XPNPEP1(10) XPO5(6) YAP(1)
YKT6(7) YWHAB(20) ZNF212(7) ZNF24(18) ZNF41(X)
ZNF44(19) ZNF574(19) ZSWIM6(5)

| | | |
|---|---|---|
| 14 | + GGCTGGTGTTAATCGGCCGAGG | ovarian colorectal |
| | (SEQ ID NO: 30) | lung brain uterus |
| | − CCTCGGCCGATTAACACCAGCC | skin kidney pancreas |
| | (SEQ ID NO: 31) | muscle lymph eye |

Targeted Genes
ARHGDIA(17) ATP7A(X) BTF3(5) CAD(2) CD59(11)
CLNS1A(11) CSNK2B(6) DAP3(1) DHTKD1(10)
DNAJB12(10) FBL(19) FLJ22688(19) GPT(8) H2AFX(11)
HDLBP(2) HSPB1(7) INSM1(20) JIK(12) LOC129138(22)
LOC144483(12) LOC145414(14) LOC158078(9)
LOC221838(7) LOC285752(6) LOC286444(X)
LOC389912(X) LOC401146(4) LOC51149(5)
LOC83468(12) MSH6(2) NFAT5(16) NME2(17) RPL3(22)
RPS2L1(20) SDBCAG84(20) SDCCAG3(9) SH3BP1(22)
SMARCA4(19) WHSC2(4) XPO5(6) ZSWIM6(5)

| | | |
|---|---|---|
| 15 | + GGGGGTGAATCGGCCGAGG | ovarian colorectal |
| | (SEQ ID NO: 32) | lung brain uterus |
| | − CCTCGGCCGATTCACCCCC | skin kidney pancreas |
| | (SEQ ID NO: 33) | muscle lymph eye |

Targeted Genes
ACTB(7) ANKRD19(9) ASB1(2) ATF4(22) C1orf26(1)
CHGB(20) COG1(17) CPS1(2) CPT1A(11) CX3CL1(16)
CYFIP2(5) ELKS(12) FMO5(1) FTL(19) G2AN(11)
GFPT1(2) GNB2L1(5) GOT2(16) GTF3C5(9) HCA127(X)
HSPA4(5) HSPA8(11) HSPCB(6) HSPCP1(4) ILVBL(19)
KDELR1(19) KIAA1917(17) LAPTM4B(8) LOC116166(15)
LOC126037(19) LOC138198(9) LOC143920(11)
LOC158714(X) LOC283820(16) LOC340600(X)
LOC388783(20) LOC390730(16) LOC391044(1)
LOC391634(4) LOC392437(X) LOC401308(7)
LOC401677(11) LOC402461(7) LOC84549(8)
LOC90850(16) LYN(8) MAP4(3) NCL(2) NICE-3(1)
NICE-4(1) NJMU-R1(17) NONO(X) ODC1(2) PHB(17)
PKD1P3(16) PKM2(15) PM5(16) PRNPIP(1) PTPN11(12)
RCN1(11) RGS4(1) RNF8(6) RPL5(1) RPN1(3)
S100A11(1) SAE1(19) SCAMP3(1) SLC25A3(12) SORD(15)
ST7(7) TIMM50(19) TM4SF11(16) U5-116KD(17)
UBE2G2(21) UCHL1(4) VARS2(6) WDR6(3) ZNF160(19)

| | | |
|---|---|---|
| 16 | + GCTGGGTGTGAATCGGCCGAGG | ovarian colorectal |
| | (SEQ ID NO: 34) | lung brain uterus |
| | − CCTCGGCCGATTCACACCCAGC | skin kidney pancreas |
| | (SEQ ID NO: 35) | muscle lymph eye |

Targeted Genes
ABCB6(2) ACTB(7) ARHGEF1(19) ATP5G2(12) AZ2(3)
BAT3(6) BCL2L14(12) BID(22) C14orf94(14)
C6orf49(6) Cab45(1) CBX7(22) CDK4(12) CHCHD2(7)
CHCHD3(7) CNOT7(8) COX5B(2) DKFZP761D0211(16)
DMAP1(1) DNPEP(2) EDARADD(1) EML2(19) ENDOG(9)
ENO1(1) ENO1P(1) FGFR4(5) FLJ11773(12)
FLJ13868(16) FLJ22169(2) FTL(19) FUS(16) G22P1(22)
GOLGA3(12) HDLBP(2) HH114(15) HIC2(22) HLA-B(6)
HSPCA(14) HSPCB(6) HSPCP1(4) HSRNAFEV(2) ILKAP(2)
IMPDH2(3) IRX4(5) ITGA1(5) K-ALPHA-1(12)
KIAA0195(17) LDHB(12) LIG1(19) LOC128439(20)
LOC130617(2) LOC134147(5) LOC136337(X)
LOC220717(2) LOC285741(6) LOC387703(10)
LOC388783(20) LOC389169(3) LOC389181(3)
LOC389424(6) LOC389787(9) LOC389901(X)
LOC391634(4) LOC392437(X) LOC392647(7)
LOC399942(11) LOC400006(12) LOC401316(7)
LOC402057(22) LOC402579(7) LOC90321(19)
LOC90850(16) LYRIC(8) MACF1(1) NAPT(17)
MGC13170(19) MGC4549(19) MRPL23(11) MVP(16)
NIFIE14(19) OSGEP(14) PA2G4(12) PDIP(16) PELO(5)
PEX10(1) PKD1-like(1) PKM2(15) POFUT1(20) PREP(6)
PRKAB1(12) PSMD3(17) PTMA(2) RPL13A(19) RPLP0(12)
RPLP0P2(11) RPS11(19) RPS17(15) RPS17P2(5)
RPS3(11) SH3YL1(2) SLC25A19(17) SNRPA(19) SNRPC(6)
SPTAN1(9) SUPT5H(19) SYNGR2(17) TH1L(20)
TIMM50(19) TPM3(1) TPT1(13) TRAF4(17) TRIM29(11)
TUBA3(12) TUBA6(12) TUFM(16) UPK3B(7) UQCRH(1)
WBSCR1(7) WDR18(19) WDR34(9)

| | | |
|---|---|---|
| 17 | + AGGTACGAGGCCGGGTGTT | ovarian colorectal |
| | (SEQ ID NO: 36) | lung brain uterus |
| | − AACACCCGGCCTCGTACCT | skin kidney pancreas |
| | (SEQ ID NO: 37) | muscle lymph eye |

Targeted Genes
ANXA2(15) ANXA2P1(4) ANXA2P2(9) AP4E1(15) ARF3(12)
ATF4(22) ATP1A1(1) ATP5A1(18) AUTL2(X) BANP(16)
C20orf43(20) C6orf69(6) CCT3(1) CCT7(2) CDT6(1)
CHCHD3(7) CLDN2(X) CLECSF9(12) CTAG3(6) DKC1(X)
E2F4(16) EEF1G(11) EIF3S8(16) EST1B(1) FLJ10349(1)
FLJ10871(8) FLJ32370(8) FRAP1(1) FSCN1(7) GAPD(12)
GNPAT(1) HMOX1(22) HNRPF(10) K-ALPHA-1(12)
KIAA1917(17) KRT18(12) LOC136337(X) LOC145414(14)
LOC158345(9) LOC284393(19) LOC285752(6)
LOC339395(1) LOC388975(2) LOC389181(3)
LOC389342(5) LOC389849(X) LOC399942(11)
LOC400966(2) LOC401369(7) LOC92755(8) LOC92755(8)
LOC94431(16) M96(1) MAP3K13(3) MGAT4B(5)
MRPL48(11) MRPL48P1(6) NFE2L1(17) NIFU(12)
NIPSNAP1(22) OK/SW-cl.56(6) P4HB(17) PCDH11X(X)
PFKM(12) PITRM1(10) PKM2(15) RNPC4(14) RPL18(19)
RPL3(22) RPLP0P2(11) RPS17P2(5) RPS3(11) RPS5(19)
RRN3(16) RYK(3) SEC24A(5) SLC25A3(12) SOD1(21)
STRN4(19) TINF2(14) TM9SF4(20) TRIM2(4) TUBA3(12)
TUBA6(12) TUBB2(9) UQCRC1(3) WBP1(2) YARS(1)
YKT6(7) ZFP106(15) ZSWIM6(5)

| | | |
|---|---|---|
| 18 | + GTGTTAATCGGCCGAGG | ovarian colorectal |
| | (SEQ ID NO: 38) | lung brain uterus |
| | − CCTCGGCCGATTAACAC | skin kidney pancreas |
| | (SEQ ID NO: 39) | muscle lymph eye |

Targeted Genes
ABCF2(7) ABHD3(18) ACOXL(2) ACTB(7) ACTG1(17)
ADCY6(12) ADRM1(20) AK2(1) AK3(1) ANP32B(9)
ANXA2P2(9) ARF4L(17) ARG2(14) ARHC(1) ARHGDIA(17)
ARPC1B(7) ARPC2(2) ARRB2(17) ASPH(8) ATP5B(12)
ATP7A(X) BACH(1) BANP(16) BAZ1A(14) BGN(X) BID(22)
BLP1(8) BTF3(5) C14orf94(14) C20orf35(20)
C22orf5(22) CAD(2) CAP1(1) CAPNS1(19) CARM1(19)
CASP4(11) CASQ1(1) CCT3(1) CD59(11) CDK2(12)
CHCHD3(7) CLDN2(X) CLECSF9(12) CLNS1A(11) CNOT7(8)
COMT(22) COQ6(14) CPE(4) CSNK2B(6) CTSB(8)
CYB5-M(16) DAP3(1) DAXX(6) DBH(9) DCI(16) DDOST(1)
DDR1(6) DDX42(17) DHCR24(1) DHTKD1(10)
DJ159A19.3(1) DKFZp434B227(3) DKFZP586J0619(7)
DNAJA1(9) DNAJB12(10) DND1(5) E2F1(20) EDARADD(1)
EEF1D(8) EEF1G(11) EI24(11) EIF2B5(3) EIF3S6IP(22)
EIF3S8(16) EMD(X) ENO1(1) ENO1P(1) ENO2(12)
EPLIN(12) ESD(13) EXT2(11) FBL(19) FBXO7(22)
FLJ10597(1) FLJ11822(17) FLJ12541(15) FLJ12949(19)
FLJ21103(11) FLJ22688(19) FLJ22843(X) FLJ27099(14)
FLJ34836(5) FLNA(X) FSCN1(7) FTL(19) FTS(16)
GAPD(12) GBF1(10) GCN5L2(17) GGA2(16) GOLGA3(12)
GOSR2(17) GPR17(2) GPT(8) GUSB(7) GYS1(19)
H2AFX(11) H3F3B(17) HADHA(2) HADHAP(4) HDGF(1)
HDLBP(2) HMOX2(16) HNRPAB(5) HNRPDL(4) HNRPU(1)
HOXA9(7) HRB2(12) HRIHFB2122(22) HS2ST1(1)
HSPB1(7) HSPCA(14) HSPCAL2(4) HSPCAL3(11)
IDH3B(20) IFI30(19) IL4I1(19) IMPDH2(3) IMUP(19)
INSIG1(7) INSM1(20) ISYNA1(19) JARID1A(12) JIK(12)
JMJD2B(19) JRK(8) JUNB(19) K-ALPHA-1(12) KHSRP(19)
KIAA0182(16) KIAA0582(2) KIAA0738(7) KIAA1614(1)
KIAA1952(9) KPNB1(17) KRT17(17) KRT19(17) KRT7(12)
KRT8(12) LDHB(12) LDHBP(X) LTMR(12) LIMS2(2)
LMNA(1) LOC113444(1) LOC115509(16) LOC129133(22)
LOC136337(X) LOC144483(12) LOC145414(14)
LOC145767(15) LOC146053(15) LOC149501(1)
LOC153027(4) LOC158078(9) LOC158473(9)
LOC192133(14) LOC220433(13) LOC221838(7)
LOC256000(4) LOC283820(16) LOC285741(6)
LOC285752(6) LOC286444(X) LOC339395(1)
LOC339736(2) LOC341056(11) LOC387851(12)
LOC388076(15) LOC388524(19) LOC388642(1)
LOC388707(1) LOC388783(20) LOC388907(22)
LOC388975(2) LOC389912(X) LOC390819(17)
LOC392437(X) LOC392647(7) LOC399942(11)
LOC399994(12) LOC400397(15) LOC400631(17)
LOC400879(22) LOC400966(2) LOC401146(4)
LOC401308(7) LOC401316(7) LOC401426(7)
LOC401504(9) LOC401972(1) LOC401987(1)
LOC402461(7) LOC402618(7) LOC51149(5)
LOC83468(12) LOC90313(17) LOC92755(8) LSM4(19)
LTBP3(11) LYPLA2(1) MAGED1(X) MAP1LC3B(16)
MAP2K1(15) MBD3(19) MCM5(22) MCM6(2) MESDC2(15)
MGC11335(16) MGC19595(19) MGC20446(11) MGC2714(11)
MGC35182(9) MIR16(16) MRPL12(17) MRPL41(9)
MRPL45(17) MRPS26(20) MSH6(2) MYBL2(20) NAP1L1(12)
NCSTN(1) NDUFA9(12) NF1(17) NFAT5(16) NIPSNAP1(22)
NME1(17) NME2(17) NONO(X) NPEPPS(17) NUDT5(10)
NUP62(19) OK/SW-cl.56(6) ORC6L(16) P2RY6(11)
PDLIM1(10) PEA15(1) PEF(1) PFKM(12) PFKP(10)
PGK1(X) PGK1P2(19) PIK4CA(22) PITRM1(10) PKM2(15)
PM5(16) PMM2(16) POLR3D(8) PPAP2C(19) PPM1G(2)
PPP1CA(11) PPT1(1) PQLC1(18) PRDX4(X) PRO1855(17)
PROCR(20) PRSS15(19) PSMC3(11) PSMC3P(9) PSMC4(19)
PTOV1(19) QDPR(4) RAB8A(19) RABEP1(17) RAC1(7)
RAC4(X) RAE1(20) RARS(5) REC14(15) RELA(11)
RNF10(12) RNF26(11) RNPS1(16) RPL22(1) RPL3(22)
RPL35A(3) RPL5(1) RPL8(8) RPLP2(11) RPN2(20)
Rpp25(15) RPS2(16) RPS2L1(20) RPS3A(4) RPS4X(X)
RPS5(19) RPS6KB2(11) RRM2(2) RRM2P3(X) RSHL1(19)
S100A16(1) SAE1(19) SARS(1) SDBCAG84(20)
SDCCAG3(9) SDHB(1) SF3B3(16) SF4(19) SH3BP1(22)
SIN3A(15) SLC25A6(X) SLC25A6(Y) SLC41A3(3)
SLC43A1(11) SMARCA4(19) SNRPN(15) SOX10(22)
SPARC(5) SPINT1(15) SRPRB(3) STRN4(19) SUPT5H(19)
TAGLN2(1) TCOF1(5) TEAD2(19) THOC3(5) TIMELESS(12)
TM4SF8(15) TM9SF4(20) TMEM4(12) TNIP1(5) TPI1(12)
TPT1(13) TRAP1(16) TUBA1(2) TUBA3(12) TUBA6(12)
U5-116KD(17) UBA2(19) UBE1(X) UCHL1(4) UPK3B(7)
UQCRC1(3) VASP(19) VCP(9) VIP32(10) WBP1(2)
WBSCR1(7) WDR1(4) WHSC2(4) XPO5(6) YARS(1)
ZDHHC12(9) ZDHHC16(10) ZNF313(20) ZNF559(19)
ZNF584(19) ZSWIM6(5)

| | | |
|---|---|---|
| 19 | + AGATGGGTACCAACTGT | ovarian colorectal |
| | (SEQ ID NO: 40) | lung brain pancreas |
| | − ACAGTTGGTACCCATCT | muscle testis eye |
| | (SEQ ID NO: 41) | |

Targeted Genes
LOC220717(2) RPLP0P2(11) RPLP0(12)

| | | |
|---|---|---|
| 20 | + CGGCTAGGTACGAGGCTGGGGT | ovarian colorectal |
| | (SEQ ID NO: 42) | lung brain uterus |
| | − ACCCCAGCCTCGTACCTAGCCG | skin kidney muscle |
| | (SEQ ID NO: 43) | lymph eye |

Targeted Genes
C5orf6(5) CASQ1(1) CCT3(1) CORO2A(9) CTAG3(6)
ENTPD8(17) FLNA(X) FOSL1(11) GAPD(12) HSPC171(16)
HSPCB(6) HSPCP1(4) KIAA0296(16) LOC388556(19)
LOC389849(X) LOC391634(4) MBTPS1(16) NARF(17)
NONO(X) PEA15(1) RER1(1) RIOK1(6) RPS3(11)
RPS9(19) RPS9P2(22) SATB1(3) SLC12A4(16) TADA3L(3)
ZNF44(19)

| | | |
|---|---|---|
| 21 | + GAGGCGGGTGTGAATCGGCCGAGG | ovarian colorectal |
| | (SEQ ID NO: 44) | brain uterus skin |
| | − CCTCGGCCGATTCACACCCGCCTC | pancreas muscle lymph |
| | (SEQ ID NO: 45) | eye |

Targeted Genes
ACTG1(17) ATP5G3(2) CCT6A(7) CN2(18) CORO1A(16)
FTL(19) HMGA1(6) HSPCB(6) HSPCP1(4) LMAN2(5)
LOC257200(2) LOC388783(20) LOC391634(4)
LOC392437(X) MGC16824(16) MGC5178(16) NASP(1)
NASPP1(8) PFDN5(12) PME-1(11) RAB5C(17) SPTAN1(9)
TERF2IP(16) UBB(17) UBBP4(17) UQCR(19)

| | | |
|---|---|---|
| 22 | + AGGTACGAGGCCGGTGT | ovarian colorectal |
| | (SEQ ID NO: 46) | brain uterus skin |
| | − ACACCGGCCTCGTACCT | kidney pancreas |
| | (SEQ ID NO: 47) | muscle lymph |

Targeted Genes
ALDH1A1(9) ARPC2(2) ATP5A1(18) BST2(19) CD79B(17)
DBH(9) DDB1(11) EIF2B5(3) EIF3S6IP(22)
EIF3S6IPP(14) ELF3(1) ENO1(1) FLJ27099(14)
G22P1(22) G6PD(X) GAPD(12) GTF3C1(16) KIAA1068(7)
KIAA1068(7b) KIAA1952(9) LOC145414(14)
LOC192133(14) LOC285741(6) LOC346085(6)
LOC387703(10) LOC387922(13) LOC388076(15)
LOC389849(X) LOC389901(X) LOC92755(8) MCM7(7)
MCSC(9) MRPL45(17) NASP(1) NASPP1(8) NDST2(10)
OAZ1(19) OK/SW-cl.56(6) RPL18(19) RPS8(1)
TAGLN2(1) TPT1(13) XRCC1(19) ZNF271(18) ZSWIM6(5)

| | | |
|---|---|---|
| 23 | + GTTAATCGGCCGAGGCGC | ovarian colorectal |
| | (SEQ ID NO: 48) | lung brain uterus |
| | − GCGCCTCGGCCGATTAAC | skin kidney pancreas |
| | (SEQ ID NO: 49) | muscle lymph |

Targeted Genes
CSNK2B(6) EIF3S6IP(22) INSIG1(7) KIAA1115(19)
KRT7(12) LOC401658(1l) LOC402057(22) LOC89958(9)
LOC92755(8) MGC3047(1) OK/SW-cl.56(6) PROCR(20)
RAN(12) RPS17(15) RPS17P2(5) SMT3H1(21) UPP1(7)
WHSC2(4)

| | | |
|---|---|---|
| 24 | + AGACCAACAGAGTTCGG | ovarian colorectal |
| | (SEQ ID NO: 50) | lung skin kidney |
| | − CCGAACTCTGTTGGTCT | pancreas |
| | (SEQ ID NO: 51) | |

Targeted Genes
novel mapping

| | | |
|---|---|---|
| 25 | + TGGCTTCGTGTCCCATGCA | breast ovarian colo- |
| | (SEQ ID NO: 52) | rectal lung skin |
| | − TGCATGGGACACGAAGCCA | muscle liver |
| | (SEQ ID NO: 53) | |

Targeted Genes
GAPD(12) GAPDL4(4) KIAA0295(15) KLHL8(4)
LOC389849(X)

| | | |
|---|---|---|
| 26 | + CCGGGTGTAAATCGGCCGA | ovarian colorectal |
| | (SEQ ID NO: 54) | brain uterus skin |
| | − TCGGCCGATTTACACCCGG | pancreas muscle lymph |
| | (SEQ ID NO: 55) | |

Targeted Genes
C19orf13(19) EIF3S6P1(6) EIF3S6(8) GNB2L1(5)
GTF2H3(12) HDAC1(1) HSPCA(14) KRT5(12) PAK1IP1(6)
PD2(19) QARS(3) SFRS10(3)

| | | |
|---|---|---|
| 27 | + GCCGGTGTGAATCGGCCGA | colorectal lung brain |
| | (SEQ ID NO: 56) | uterus skin kidney |
| | − TCGGCCGATTCACACCGGC | pancreas muscle |
| | (SEQ ID NO: 57) | |

Targeted Genes
ARHC(1) ATP7B(13) BCAP31(X) C20orf35(20)
CTDSP2(12) EBNA1BP2(1) FLJ10737(1) FLJ20254(2)
G22P1(22) HDLBP(2) HMGN2(1) HS3ST4(16)
HSA272196(17) HSPC117(22) LCP1(13) LOC339395(1)
LOC387703(10) LOC389901(X) MGC11242(17) MRPL51(12)
NAP1L1(12) NDUFV1(11) POLDIP2(17) PSMB1(6)
SIRT2(19) SQSTM1(5) SRPR(11) STK25(2) SV2C(5)
TAGLN2(1) TJP1(15) XRCC1(19)

| | | |
|---|---|---|
| 28 | + TCATGATGGTGTATCGATGA | ovarian colorectal |
| | (SEQ ID NO: 58) | lung brain skin bone |
| | − TCATCGATACACCATCATGA | |
| | (SEQ ID NO: 59) | |

Targeted Genes
JIK(12) LOC400963(2) LOC91561(11) LOC286444(X)

| | | |
|---|---|---|
| 29 | + GCTCGGTGTTAATCGGCCGA | ovarian colorectal |
| | (SEQ ID NO: 60) | brain uterus skin |
| | − TCGGCCGATTAACACCGAGC | pancreas lymph eye |
| | (SEQ ID NO: 61) | |

Targeted Genes
CASP4(11) GGA2(16) HRIHFB2122(22) INSIG1(7)
KHSRP(19) LOC388642(1) LOC400879(22) PRDX4(X)
RPS2(16) SDHB(1) SLC25A6(X) SLC25A6(Y) TPI1(12)
TRAP1(16) VIP32(10)

| | | |
|---|---|---|
| 30 | + TGGGGTTAATCGGCCGAGG | ovarian colorectal |
| | (SEQ ID NO: 62) | lung uterus skin |
| | − CCTCGGCCGATTAACCCCA | pancreas lymph eye |
| | (SEQ ID NO: 63) | |

Targeted Genes
ADRBK1(11) BCKDK(16) LOC220717(2) MGC3329(17)
MRPL15(8) QARS(3) RPLP0(12) RPLP0P2(11) RPS9(19)
RPS9P2(22) SPATA11(19) SRM(1) TADA3L(3) TUFM(16)

| | | |
|---|---|---|
| 31 | + AGGCCGGTGTTAATCGGCCGA | ovarian colorectal |
| | (SEQ ID NO: 64) | lung brain uterus |
| | − TCGGCCGATTAACACCGGCCT | skin kidney pancreas |
| | (SEQ ID NO: 65) | lymph |

Targeted Genes
ACTG1(17) AK3(1) ANXA2P2(9) ARPC2(2) ATP5B(12)
CPE(4) DBH(9) DCI(16) DHCR24(1) DJ159A19.3(1)
EEF1D(8) ENO1(1) GOLGA3(12) HADHA(2) HADHAP(4)
HIP-55(7) HNRPU(1) JMJD2B(19) K-ALPHA-1(12)
KIAA1952(9) LOC145414(14) LOC158473(9)
LOC285741(6) LOC387851(12) LOC388524(19)
LOC388707(1) LOC392647(7b) LOC399942(11)
LOC399994(12) LOC401316(7) LOC401504(9)
LOC401987(1) MRPL45(17) NF1(17) NME1(17)
PRSS15(19) RABEP1(17) SOX10(22) SRPRB(3) TAGLN2(1)
TPT1(13) TUBA3(12) TUBA6(12) VCP(9) WBSCR1(7)
ZSWIM6(5)

| | | |
|---|---|---|
| 32 | + TGGTGAATCGGCCGAGGGT | ovarian colorectal |
| | (SEQ ID NO: 66) | brain uterus skin |
| | − ACCCTCGGCCGATTCACCA | kidney pancreas lymph |
| | (SEQ ID NO: 67) | |

Targeted Genes
ACADS(12) C20orf149(20) DCTN3(9) DPYSL3(5)
EIF3S1(15) IPO4(14) KIAA0152(12) LOC388556(19)
LOC401092(3) PRDX5(11) PSMF1(20) RAB11A(15)
RPL10(X) RPS9(19) RPS9P2(22) STXBP2(19) ZNF3(7)
ZNF-U69274(3)

| | | |
|---|---|---|
| 33 | + AGCAAGTATGACAACAGC | colorectal lung |
| | (SEQ ID NO: 68) | cervix skin pancreas |
| | − GCTGTTGTCATACTTGCT | muscle |
| | (SEQ ID NO: 69) | |

Targeted Genes
GAPD(12) LOC389849(X)

| | | |
|---|---|---|
| 34 | + CTTAAACCAAGCTAGCC | colorectal prostate |
| | (SEQ ID NO: 70) | brain skin bone |
| | − GGCTAGCTTGGTTTAAG | testis eye |
| | (SEQ ID NO: 71) | |

Targeted Genes
LOC143371(10) LOC150554(2) LOC158383(9) YWHAZ(8)

| | | |
|---|---|---|
| 35 | + CAGTCTACATCACGTGG | colorectal lung |
| | (SEQ ID NO: 72) | cervix brain kidney |
| | − CCACGTGATGTAGACTG | lymph liver eye |
| | (SEQ ID NO: 73) | |

Targeted Genes
LOC359792(Y) LOC400039(12) PCDH11X(X) PCDH11Y(Y)

| | | |
|---|---|---|
| 36 | + AATCTCCTGTTACACTCA | ovarian colorectal |
| | (SEQ ID NO: 74) | brain epid testis |
| | − TGAGTGTAACAGGAGATT | |
| | (SEQ ID NO: 75) | |

Targeted Genes
LOC146909 (17)

| | | |
|---|---|---|
| 37 | + GCCCAAGGAACCCCCTT | ovarian colorectal |
| | (SEQ ID NO: 76) | lung skin testis |
| | − AAGGGGGTTCCTTGGGC | liver eye |
| | (SEQ ID NO: 77) | |

Targeted Genes
ABCC3(17) CHCHD3(7) EEF1G(11) LOC136337(X)

| | | |
|---|---|---|
| 38 | + GGCTAGGACGAGGCCGGG | colorectal brain skin |
| | (SEQ ID NO: 78) | kidney pancreas |
| | − CCCGGCCTCGTCCTAGCC | muscle lymph |
| | (SEQ ID NO: 79) | |

Targeted Genes
ATP6V1E1(22) CCT4(2) CHGB(20) DHX9(1) EIF3S8(16)
LOC343515(1) MAP2K2(19) NDUFA9(12) NDUFA9P1(22)
SCARB1(12)

| | | |
|---|---|---|
| 39 | + GAGAAGGTTCCCGGGAA | colorectal lung |
| | (SEQ ID NO: 80) | pancreas lymph liver |
| | − TTCCCGGGAACCTTCTC | eye |
| | (SEQ ID NO: 81) | |

Targeted Genes
CHCHD3(7) EEF1G(11) LOC136337(X) MGC10471(19)

| | | |
|---|---|---|
| 40 | + GTGTTACTCGGCCGAGG | colorectal lung brain |
| | (SEQ ID NO: 82) | uterus skin kidney |
| | − CCTCGGCCGAGTAACAC | pancreas muscle |
| | (SEQ ID NO: 83) | |

Targeted Genes
ACLY(17) ADAR(1) ALDH1A1(9) C12orf10(12) GNAI2(3)
K-ALPHA-1(12) LMNB2(19) LOC400671(19) PPIE(1)
RYK(3) TTYH3(7) TUBA3(12) TUBA6(12)

| | | |
|---|---|---|
| 41 | + TTGAATCGGCCGAGGGTG | ovarian colorectal |
| | (SEQ ID NO: 84) | lung brain pancreas |
| | − CACCCTCGGCCGATTCAA | muscle eye |
| | (SEQ ID NO: 85) | |

Targeted Genes
CINP(14) COTL1(16) FLJ39075(16) GNB2L1(5)
KRT19(17) KRT4(12) LOC92305(4) MCSC(9) PCNT1(17)
PH-4(3) RPL8(8) ZNF337(20)

| | | |
|---|---|---|
| 42 | + GCCGGGTGGTGAATCGG | ovarian colorectal |
| | (SEQ ID NO: 86) | brain uterus skin |
| | − CCGATTCACCACCCGGC | kidney muscle |
| | (SEQ ID NO: 87) | |

Targeted Genes
ACTG1(17) CHCHD3(7) DFFA(1) DPYSL3(5) PRDX5(11)
SYMPK(19) TSPAN-1(1) ZDHHC16(10)

| | | |
|---|---|---|
| 43 | + GCCGGTGGTTAATCGGC | colorectal lung brain |
| | (SEQ ID NO: 88) | uterus skin kidney |
| | − GCCGATTAACCACCGGC | pancreas |
| | (SEQ ID NO: 89) | |

Targeted Genes
C6orf109(6) CFL1(11) FLJ30934(11) GALNT2(1)
K-ALPHA-1(12) LOC145414(14) LOC285752(6)
LOC399942(11) LOC56931(19) PCDH18(4) PSMC3(11)
RPL3(22) SARS(1) STK19(6) TCF7L1(2) TETRAN(4)
TUBA3(12) TUBA6(12)

| | | |
|---|---|---|
| 44 | + GGGCGCAGCGACATCAG | colorectal prostate |
| | (SEQ ID NO: 90) | lung adrenal pancreas |
| | − CTGATGTCGCTGCGCCC | lymph eye |
| | (SEQ ID NO: 91) | |

Targeted Genes
TREX2 (X)

| | | |
|---|---|---|
| 45 | + GCTATTAGCAGATTGTGT | colorectal lung |
| | (SEQ ID NO: 92) | kidney muscle testis |
| | − ACACAATCTGCTAATAGC | eye |
| | (SEQ ID NO: 93) | |

Targeted Genes
LOC399942(11) K-ALPHA-1(12) TUBA3(12) TUBA6(12)

| | | |
|---|---|---|
| 46 | + TGTTAATCTCCTGTTACACTCA | ovarian colorectal |
| | (SEQ ID NO: 94) | brain epid testis |
| | − TGAGTGTAACAGGAGATTAACA | liver |
| | (SEQ ID NO: 95) | |

Targeted Genes
LOC146909(17)

| | | |
|---|---|---|
| 47 | + CCACCGCACCGTTGGCC | ovarian colorectal |
| | (SEQ ID NO: 96) | cervix skin kidney |
| | − GGCCAACGGTGCGGTGG | testis |
| | (SEQ ID NO: 97) | |

Targeted Genes
FBXW5(9)

| | | |
|---|---|---|
| 48 | + ACCTGGAGCCCTCTGAT | colorectal lung skin |
| | (SEQ ID NO: 98) | kidney muscle liver |
| | − ATCAGAGGGCTCCAGGT | |
| | (SEQ ID NO: 99) | |

Targeted Genes
LOC399942(11) K-ALPHA-1(12) TUBA3(12) TUBA6(12)

| | | |
|---|---|---|
| 49 | + TCAGACAAACACAGATCG | colorectal prostate |
| | (SEQ ID NO: 100) | lung brain muscle |
| | − CGATCTGTGTTTGTCTGA | |
| | (SEQ ID NO: 101) | |

Targeted Genes
LOC285900(7) DGKI(7) LOC402525(7b) LOC388460(18)
RPL6(12)

| | | |
|---|---|---|
| 50 | + GAGAATACTGATTGAGACCTA | ovarian colorectal |
| | (SEQ ID NO: 102) | skin kidney lymph |
| | − TAGGTCTCAATCAGTATTCTC | testis |
| | (SEQ ID NO: 103) | |

Targeted Genes
LOC92755(8) OK/SW-cl.56(6)

| | | |
|---|---|---|
| 51 | + CCAGCCAGCACCCAGGC | colorectal gall skin |
| | (SEQ ID NO: 104) | pancreas lymph |
| | − GCCTGGGTGCTGGCTGG | |
| | (SEQ ID NO: 105) | |

Targeted Genes
ATP5A1(18) FLJ10101(9) IL9R(X) IL9R(Y)
LOC392325(9) LOC400481(16) RELA(11)

| | | |
|---|---|---|
| 52 | + TAGACCAACAGAGTTCC | colorectal lung skin |
| | (SEQ ID NO: 106) | kidney muscle liver |
| | − GGAACTCTGTTGGTCTA | |
| | (SEQ ID NO: 107) | |

Targeted Genes
novel mapping

| | | |
|---|---|---|
| 53 | + CTAGGTACGAGGCTGGGTTTT | colorectal lung |
| | (SEQ ID NO: 108) | uterus skin muscle |
| | − AAAACCCAGCCTCGTACCTAG | lymph |
| | (SEQ ID NO: 109) | |

Targeted Genes
ACTG1(17) LOC81691(16) PSAP(10) SFRS2(17)

| | | |
|---|---|---|
| 54 | + CGAGGCGGGTGTTAATCGGCC | colorectal lung brain |
| | (SEQ ID NO: 110) | skin pancreas lymph |
| | − GGCCGATTAACACCCGCCTCG | eye |
| | (SEQ ID NO: 111) | |

Targeted Genes
ACTB(7) ADCY6(12) BID(22) EIF3S6IP(22) EIF3S8(16)
K-ALPHA-1(12) MRPL12(17) PDLIM1(10) RARS(5)
RPN2(20) S100A16(1) TUBA1(2)

| | | |
|---|---|---|
| 55 | + AAGGCTAGGTAGAGGCTG | ovarian colorectal |
| | (SEQ ID NO: 112) | brain pancreas muscle |
| | − CAGCCTCTACCTAGCCTT | eye |
| | (SEQ ID NO: 113) | |

Targeted Genes
ANP32B(9) C20orf14(20) CAD(2) COL14A1(8)
CTNNBL1(20) DOK4(16) ENO1(1) FLJ22301(1) HSPCB(6)
HSPCP1(4) K-ALPHA-1(12) LOC339395(1) LOC391634(4)
LOC400397(15) PKM2(15) RACGAP1(12) STATIP1(18)
VASP(19)

| | | |
|---|---|---|
| 56 | + CATGGCCATGCTGTGCA | colorectal uterus |
| | (SEQ ID NO: 114) | skin testis |
| | − TGCACAGCATGGCCATG | |
| | (SEQ ID NO: 115) | |

Targeted Genes
DNPEP(2) MATP(5)

| | | |
|---|---|---|
| 57 | + AGGTACGAGGCCGGTGTTAATCGG | ovarian colorectal |
| | CCGA | lung brain kidney |
| | (SEQ ID NO: 116) | lymph |
| | − TCGGCCGATTAACACCGGCCTCGT | |
| | ACCT | |
| | (SEQ ID NO: 117) | |

Targeted Genes
ARPC2(2) DBH(9) ENO1(1) KIAA1952(9) LOC145414(14)
LOC285741(6) MRPL45(17) TAGLN2(1) TPT1(13)
ZSWIM6(5)

| | | |
|---|---|---|
| 59 | + TGCTGCCCTCAATGGTC | colorectal lung |
| | (SEQ ID NO: 118) | cervix skin muscle |
| | − GACCATTGAGGGCAGCA | eye |
| | (SEQ ID NO: 119) | |

Targeted Genes
novel mapping

| | | |
|---|---|---|
| 60 | + AGGCCGGTGGTTAATCGGCCGAGG | colorectal brain |
| | (SEQ ID NO: 120) | uterus skin kidney |
| | − CCTCGGCCGATTAACCACCGGCCT | pancreas |
| | (SEQ ID NO: 121) | |

Targeted Genes
C6orf109(6) GALNT2(1) LOC145414(14) LOC285752(6)
LOC56931(19) PCDH18(4) PSMC3(11) RPL3(22) STK19(6)
TETRAN(4)

| | | |
|---|---|---|
| 61 | + GAGGCCGGTGGTTAATCGGCCGAG | colorectal brain |
| | (SEQ ID NO: 122) | uterus skin kidney |
| | − CTCGGCCGATTAACCACCGGCCTC | pancreas |
| | (SEQ ID NO: 123) | |

Targeted Genes
C6orf109(6) LOC145414(14) LOC285752(6) LOC56931(19)
PCDH18(4) PSMC3(11) RPL3(22) STK19(6) TETRAN(4)

| | | |
|---|---|---|
| 62 | + GCTAGGTACGAGGCTGGGTTTT | colorectal lung |
| | (SEQ ID NO: 124) | uterus skin muscle |
| | − AAAACCCAGCCTCGTACCTAGC | lymph |
| | (SEQ ID NO: 125) | |

Targeted Genes
ACTG1(17) PSAP(10) SFRS2(17)

| | | |
|---|---|---|
| 63 | + AACATACGGCTAGGTACGA | ovarian colorectal |
| | (SEQ ID NO: 126) | brain uterus lymph |
| | − TCGTACCTAGCCGTATGTT | eye |
| | (SEQ ID NO: 127) | |

Targeted Genes
CIZ1(9) FLJ20203(1) FLJ23416(17) MGC3162(12)
MSF(17) SWAP70(11) YAP(1)

| | | |
|---|---|---|
| 64 | + GGTGGTAATCGGACGAGG | colorectal lung brain |
| | (SEQ ID NO: 128) | uterus skin muscle |
| | − CCTCGTCCGATTACCACC | |
| | (SEQ ID NO: 129) | |

Targeted Genes
AKT1(14) CHGA(14) CHRNA3(15) EMS1(11) FLJ20244(19)
FLJ22169(2) GNB2L1(5) LOC130617(2) LOC284393(19)
LOC347422(X) LOC388642(1) LOC389342(5) SLC4A2(7)
TIMM17B(X) TPI1(12) YKT6(7)

| | | |
|---|---|---|
| 65 | + GGGTGATCGGACGAGGC | ovarian colorectal |
| | (SEQ ID NO: 130) | lung brain pancreas |
| | − GCCTCGTCCGATCACCC | eye |
| | (SEQ ID NO: 131) | |

Targeted Genes
ACTG1(17) ANKRD19(9) DNAJB11(3) EEF1D(8) HSPCA(14)
HSPCAL2(4) HSPCAL3(11) LOC126037(19) LOC399704(6)
RABAC1(19)

| | | |
|---|---|---|
| 66 | + ACATGCCTAGGGTTCAA | colorectal lung |
| | (SEQ ID NO: 132) | cervix pancreas |
| | − TTGAACCCTAGGCATGT | testis eye |
| | (SEQ ID NO: 5) | |

Targeted Genes
EEF1A1(6) LOC401146(4)

### Example 11

### Cellular Genes with Targetable Apoptotic Sequences

Further computational analysis was used to compare selected Apoptotic Sequences with gene databases to identify genes that frequently contained Apoptotic Sequences in patients with cancer. These genes may serve as a target for inducing apoptosis in cancer cells even though most or all are not oncogenes. These genes may only be expressed in cancer cells, or treatment may focus on variations of these genes having only apoptotic sequences.

The genes shown in Table 6 contain Apoptotic Sequences. Table 6 shows the number of recorded occurrences in cancer patients of known Apoptotic Sequences contained in a gene, and the gene name and chromosomal location in the human genome.

TABLE 6
Occurrence of Apoptotic Sequences in Genes

| Recorded Occurrence | Gene Name (Chromosome) |
|---|---|
| 45 | [GAPD(12)] |
| 38 | [ACTG1(17)] |
| 34 | [PKM2(15)] |
| 33 | [K-ALPHA-K12)] |
| 28 | [HSPCB(6)] |
| 27 | [ENO1(1)] |
| 25 | [OK/SW-cl.56(6)] |
| 24 | [RPL3(22)] |
| 24 | [GNB2L1(5)] |
| 24 | [MAP3K13(3)] |
| 23 | [CHCHD3(7)] |
| 21 | [HSPCP1(4)] |
| 20 | [RPL8(8)] |
| 20 | [CCT3(1)] |
| 20 | [ACTB(7)] |
| 18 | [ALDOA(16)] |
| 18 | [PPP4C(16)] |
| 16 | [SLC25A6(X & Y)] |
| 16 | [SLC25A3(12)] |
| 16 | [IMPDH2(3)] |
| 16 | [ARF1(1)] |
| 15 | [ASPH(8)] |
| 15 | [TK1(17)] |
| 15 | [CCT7(2)] |
| 14 | [TUBA6(12)] |
| 14 | [RPS2(16)] |
| 14 | [RAN(12)] |
| 14 | [RANP1(6)] |
| 14 | [EIF3S8(16)] |
| 13 | [RPL4(15)] |
| 13 | [RPLP0(12)] |
| 13 | [SHMT2(12)] |
| 12 | [LDHB(12)] |
| 12 | [KRT18(12)] |
| 12 | [FBL(19)] |
| 12 | [ATP5A1(18)] |
| 11 | [SNRPB(20)] |
| 11 | [RPL13A(19)] |
| 11 | [RPL13(16)] |
| 11 | [P4HB(17)] |
| 11 | [NONO(X)] |
| 11 | [KRT19(17)] |
| 11 | [BSG(19)] |
| 11 | [ATP5B(12)] |
| 11 | [ARPC1B(7)] |
| 10 | [TUBB2(9)] |
| 10 | [TAGLN2(1)] |
| 10 | [SQSTM1(5)] |
| 10 | [MGAT4B(5)] |
| 10 | [MCM7(7)] |
| 10 | [RPLP0P2(11)] |
| 10 | [E1B-AP5(19)] |
| 10 | [CTSD(11)] |
| 10 | [CST3(20)] |
| 10 | [CCNB1(5)] |
| 10 | [ANXA2P2(9)] |
| 9 | [RNASEH2A(19)] |
| 9 | [KIF13B(8)] |
| 9 | [HSPB1(7)] |
| 9 | [HDLBP(2)] |
| 9 | [DDOST(1)] |
| 8 | [eIF3k(19)] |
| 8 | [UQCRCK3)] |
| 8 | [TXNDC5(6)] |
| 8 | [TEGT(12)] |
| 8 | [RPS6KB2(11)] |
| 8 | [PPP1CA(11)] |
| 8 | [PSMB5(14)] |
| 8 | [PSAP(10)] |
| 8 | [PRSS15(19)] |
| 8 | [PPM1G(2)] |
| 8 | [PGD(1)] |
| 8 | [MBD3(19)] |
| 8 | [LMNA(1)] |
| 8 | [ITGB4BP(20)] |
| 8 | [HNRPC(14)] |
| 8 | [FOXM1(12)] |
| 8 | [CALM2(2)] |
| 7 | [UPP1(7)] |
| 7 | [TUBB(6)] |
| 7 | [TUBA3(12)] |
| 7 | [TTC1(5)] |
| 7 | [SLC1A5(19)] |
| 7 | [RUVBL1(3)] |
| 7 | [OPRS1(9)] |
| 7 | [NUP98(11)] |
| 7 | [NPAS3(14)] |
| 7 | [NICE-3(1)] |
| 7 | [NICE-4(1)] |
| 7 | [MSI2(17)] |
| 7 | [LGALS3BP(17)] |
| 7 | [KIAA1841(2)] |
| 7 | [KIAA0934(10)] |
| 7 | [HNRPD(4)] |
| 7 | [HDAC7A(12)] |
| 7 | [HBXAP(11)] |
| 7 | [GTF3A(13)] |
| 7 | [GNAI2(3)] |
| 7 | [EEF2(19)] |
| 7 | [Cab45(1)] |
| 7 | [CLU(8)] |
| 7 | [CHTF18(16)] |
| 7 | [CCT5(5)] |
| 7 | [ATP5F1(1)] |
| 7 | [ATP1A1(1)] |
| 7 | [KRT8(12)] |
| 7 | [ANP32B(9)] |
| 7 | [AKT1(14)] |
| 7 | [ABCC3(17)] |
| 6 | [ZNF500(16)] |
| 6 | [WT1(11)] |
| 6 | [VIPR2(7)] |
| 6 | [VIM(10)] |
| 6 | [UROD(1)] |
| 6 | [TTN(2)] |
| 6 | [TPD52(8)] |
| 6 | [TKT(3)] |
| 6 | [TCOF1(5)] |
| 6 | [TCF3(19)] |
| 6 | [STMN1(1)] |
| 6 | [RPS16(19)] |
| 6 | [PRPF31(19)] |
| 6 | [POLD2(7)] |
| 6 | [PHGDH(1)] |
| 6 | [NR4A3(9)] |
| 6 | [NASP(1)] |
| 6 | [MYBL2(20)] |
| 6 | [MTA3(2)] |
| 6 | [MGC19595(19)] |
| 6 | [MGC10200(18)] |
| 6 | [MDS027(3)] |
| 6 | [MCM5(22)] |
| 6 | [MAGEE1(X)] |
| 6 | [LTBR(12)] |
| 6 | [KPNA2(17)] |
| 6 | [KIAA1076(12)] |
| 6 | [KIAA0117(1)] |
| 6 | [JIK(12)] |
| 6 | [HSPC142(19)] |
| 6 | [HMGA1(6)] |
| 6 | [HDGF(1)] |
| 6 | [HDAC4(2)] |
| 6 | [H3F3B(17)] |
| 6 | [GUK1(1)] |
| 6 | [GSN(9)] |
| 6 | [FUT10(8)] |
| 6 | [FTL(19)] |
| 6 | [FH(1)] |
| 6 | [FBF-1(17)] |
| 6 | [FASN(17)] |
| 6 | [FARSLA(19)] |
| 6 | [ESDN(3)] |
| 6 | [ERP70(7)] |
| 6 | [EEF1G(11)] |
| 6 | [DustyPK(1)] |
| 6 | [DRIM(12)] |
| 6 | [CUGBP2(10)] |
| 6 | [CS(12)] |
| 6 | [CORO1A(16)] |
| 6 | [COL1A2(7)] |
| 6 | [CLK3(15)] |
| 6 | [CKAP1(19)] |
| 6 | [CD74(5)] |
| 6 | [BCKDK(16)] |
| 6 | [BAT1(6)] |
| 6 | [ATP11A(13)] |
| 6 | [ARHGDIA(17)] |
| 6 | [APOE(19)] |
| 6 | [AP2M1(3)] |
| 6 | [ADIPOR2(12)] |
| 5 | [ZNF9(3)] |
| 5 | [ZNF30(19)] |
| 5 | [YWHAZ(8)] |
| 5 | [WIT-1(11)] |
| 5 | [VCP(9)] |
| 5 | [USP22(17)] |
| 5 | [UBE2C(20)] |
| 5 | [TYR(11)] |
| 5 | [TRO(X)] |
| 5 | [TRIM28(19)] |
| 5 | [TRAP1(16)] |
| 5 | [TNFSF4(1)] |
| 5 | [TIGD5(8)] |
| 5 | [THAP6(4)] |
| 5 | [STARD7(2)] |
| 5 | [SNRPA(19)] |
| 5 | [SLC7A5(16)] |
| 5 | [SLC5A10(17)] |
| 5 | [SIPA1L2(1)] |
| 5 | [SELH(11)] |
| 5 | [SDHA(5)] |
| 5 | [RPS9(19)] |
| 5 | [RPS9P2(22)] |
| 5 | [RPS6(9)] |
| 5 | [RPL14(3)] |
| 5 | [RASSF1(3)] |
| 5 | [RAI1(17)] |
| 5 | [RAB7(3)] |
| 5 | [PYCR1(17)] |
| 5 | [PTTG1IP(21)] |
| 5 | [PTDSR(17)] |
| 5 | [PSMD2(3)] |
| 5 | [PSK(16)] |
| 5 | [PRNP(20)] |
| 5 | [POLR2E(19)] |
| 5 | [POGZ(1)] |
| 5 | [PLP2(X)] |
| 5 | [PCDH7(4)] |
| 5 | [OAZ1(19)] |
| 5 | [NRXN1(2)] |
| 5 | [NEUGRIN(15)] |
| 5 | [NDRG1(8)] |
| 5 | [MYEOV(11)] |
| 5 | [MYCN(2)] |
| 5 | [MRPS28(8)] |
| 5 | [MOV10(1)] |
| 5 | [MLPH(2)] |
| 5 | [MLF2(12)] |
| 5 | [MGC4659(14)] |
| 5 | [MGC45866(15)] |
| 5 | [MGC3731(22)] |
| 5 | [MGC22960(1)] |
| 5 | [MFI2(3)] |
| 5 | [MCM2(3)] |
| 5 | [MAGED1(X)] |
| 5 | [TPI1(12)] |
| 5 | [TPT1(13)] |
| 5 | [KTN1(14)] |
| 5 | [KRT17(17)] |
| 5 | [KPNB1(17)] |
| 5 | [IL4I1(19)] |
| 5 | [NUP62(19)] |
| 5 | [HRMT1L2(19)] |
| 5 | [HRI(7)] |
| 5 | [HPCAL1(2)] |
| 5 | [HIC(7)] |
| 5 | [HAND1(5)] |
| 5 | [GYS1(19)] |
| 5 | [GTPBP5(20)] |
| 5 | [GOLGA2(9)] |
| 5 | [GNAO1(16)] |
| 5 | [GLTSCR2(19)] |
| 5 | [GFAP(17)] |
| 5 | [GALNT2(1)] |
| 5 | [FZD9(7)] |
| 5 | [FRAS1(4)] |
| 5 | [FBXO10(9)] |
| 5 | [FBXL11(11)] |
| 5 | [DSTN(20)] |
| 5 | [NPM1(5)] |
| 5 | [DHX37(12)] |
| 5 | [DDB1(11)] |
| 5 | [CSNK1E(22)] |
| 5 | [COG5(7)] |
| 5 | [CLTA(9)] |
| 5 | [CENPF(1)] |
| 5 | [CD81(11)] |
| 5 | [CCT6A(7)] |
| 5 | [CBS(21)] |
| 5 | [CASP8(2)] |
| 5 | [CAD(2)] |
| 5 | [BCL9(1)] |
| 5 | [BAZ2A(12)] |
| 5 | [AUTS2(7)] |
| 5 | [ATP6V0C(16)] |
| 5 | [ATP5G3(2)] |
| 5 | [AP2S1(19)] |
| 5 | [ANKS1(6)] |
| 5 | [AKR1B1(7)] |
| 5 | [AFAP(4)] |
| 5 | [MGC10981(4)] |
| 5 | [ADNP(20)] |
| 5 | [ACO2(22)] |
| 5 | [ABCD1(X)] |

Molecules used to target these genes may include small molecules or nucleic acids, including all forms of DNA and RNA, particularly treated forms. These molecules may be in any formulation that is pharmaceutically acceptable. Some formulations may aid in delivery or therapeutic effect.

The safety and efficacy of a given gene-targeting molecule based on an Apoptotic Sequence in any given formulation may be tested using available methods, non-limiting embodiments of which are described in the above paragraphs. In one particular example, it may be tested by providing the molecule in vitro to healthy cells and cancer cells of similar origin and selecting only molecules that kill many cancer cells while leaving the healthy cells substantially unharmed.

In vivo tests may also be performed in appropriate models. In one example, because healthy cell death is a direct reflection of toxicity, mice may be administered a molecule or not and observed for changes in physiology or behavior. For example, if the molecule is a nucleic acid, it may be administered in an amount of approximately 5 mg to 50 mg per 1 kg of body weight, in particular no more than 25 mg per 1 kg of body weight.

Various publications are cited herein, the contents of which are incorporated by reference herein in their entireties.

While embodiments of this disclosure have been depicted, described, and are defined by reference to specific example embodiments of the disclosure, such references do not imply a limitation on the disclosure, and no such limitation is to be inferred. The subject matter disclosed is capable of considerable modification, alteration, and equivalents in form and function, as will occur to those ordinarily skilled in the pertinent art and having the benefit of this disclosure. The depicted and described embodiments of this disclosure are examples only, and are not exhaustive of the scope of the disclosure. For example, one or ordinary skill in the art will recognize that, in many situations, nucleic acids complementary to the Apoptotic Sequences specified herein will themselves be Apoptotic Sequences.

1. A targeting agent comprising a first isolated nucleic acid molecule that specifically hybridizes to a second nucleic acid having an Apoptotic Sequence and located in a cancer cell, wherein delivery of the targeting agent into the cancer cell results in induction of cell death in the cancer cell.

2. The targeting agent of claim 1, wherein the first isolated nucleic acid comprises a DNA molecule.

3. The targeting agent of claim 1, wherein the first isolated nucleic acid comprises an RNA molecule.

4. The targeting agent of claim 1, wherein the second nucleic acid comprises a DNA molecule.

5. The targeting agent of claim 1, wherein the second nucleic acid comprises a RNA molecule.

6. The targeting agent of claim 1, wherein the first isolated nucleic acid comprises inter-nucleotide linkages other than phosphodiester bonds.

7. The targeting agent of claim 6, wherein the inter-nucleotide linkages comprise a phosphorothioate, a methylphosphonate, a methylphosphodiester, a phosphorodithioate, a phosphoramidate, a phosphotriester, or a phosphate ester linkage.

8. The targeting agent of claim 1, wherein the first isolated nucleic acid molecule specifically hybridizes to second nucleic acid molecule comprising a sequence selected from the group consisting of: SEQ.ID.NO:1, SEQ.ID.NO:2, SEQ.ID.NO:3, SEQ.ID.NO:4, SEQ.ID.NO:5, SEQ.ID.NO:6, SEQ.ID.NO:7, and sequences complementary thereto.

9. A composition comprising:
a first isolated nucleic acid molecule that specifically hybridizes to a second nucleic acid having an Apoptotic Sequence and located in a cancer cell; and
a pharmaceutically acceptable carrier,
wherein delivery of the targeting agent into the cancer cell results in induction of cell death in the cancer cell.

10. The targeting agent of claim 9, wherein the first isolated nucleic acid molecule specifically hybridizes to second nucleic acid molecule comprising a sequence selected from the group consisting of: SEQ.ID.NO:1, SEQ.ID.NO:2, SEQ.ID.NO:3, SEQ.ID.NO:4, SEQ.ID.NO:5, SEQ.ID.NO:6, SEQ.ID.NO:7, and sequences complementary thereto.

11. The targeting agent of claim 9, further comprising a coating to protect the first isolated nucleic acid from the action of alkali, acid and other natural conditions that may cause degradation of the nucleic acid.

12. A method of killing a cancer cell comprising:
administering to the cancer cell a first isolated nucleic acid molecule that specifically hybridizes to a second nucleic acid having an Apoptotic Sequence and located in the cancer cell; and
inducing cell death in the cancer cell via the first isolated nucleic acid.

13. The method of claim 12, wherein the first isolated nucleic acid molecule specifically hybridizes to second nucleic acid molecule comprising a sequence selected from the group consisting of: SEQ.ID.NO:1, SEQ.ID.NO:2, SEQ.ID.NO:3, SEQ.ID.NO:4, SEQ.ID.NO:5, SEQ.ID.NO:6, SEQ.ID.NO:7, and sequences complementary thereto.

14. The method of claim 12, wherein cancer cell death comprises apoptosis.

15. The method of claim 12, comprising administering the first isolated nucleic acid to the cancer cell in a subject with cancer.

16. The method of claim 15, wherein the cancer cell is a breast cancer.

17. The method of claim 15, wherein the cancer cell is a a colon cancer.

18. The method of claim 15, wherein the cancer cell is a lung cancer.

19. The method of claim 15, wherein the cancer cell is a brain cancer.

20. The method of claim 15, wherein the cancer cell is a glioblastoma.

21. The method of claim 15, wherein the cancer cell is a medulloblastoma.

22. The method of claim 15, wherein the cancer cell is an ovarian cancer.

23. The method of claim 15, comprising administering the first isolated nucleic acid in a pharmaceutically acceptable carrier.
